(19) United States
(12) Patent Application Publication
(10) Pub. No.: US 2025/0283114 A1
(43) Pub. Date: Sep. 11, 2025

(54) MODIFIED STRAINS FOR IMPROVED SECRETION OF RECOMBINANT PROTEINS

(71) Applicant: BOLT THREADS, INC., Emeryville, CA (US)

(72) Inventor: Thomas Stevens, Emeryville, CA (US)

(21) Appl. No.: 19/191,916

(22) Filed: Apr. 28, 2025

Related U.S. Application Data
(63) Continuation of application No. 18/178,275, filed on Mar. 3, 2023, now Pat. No. 12,286,640.
Application No. 18/178,275 is a continuation of application No. 16/415,605, filed on May 17, 2019, now Pat. No. 11,634,729.
(60) Provisional application No. 62/673,001, filed on May 17, 2018.

(51) Int. Cl. C12N 15/90; C07K 16/14; C12N 15/62

(52) U.S. Cl. CPC C12N 15/905; C07K 16/14; C12N 15/625

(57) ABSTRACT

Disclosed herein are modified strains for improving secretion of recombinantly expressed products secreted from a host organism with improved growth and productivity characteristics, as well as methods of using the modified strains.

### CROSS-REFERENCE TO RELATED APPLICATIONS

This application is a continuation of U.S. patent application Ser. No. 18/178,275, filed Mar. 3, 2023, which is a continuation of U.S. patent application Ser. No. 16/415,605, filed May 17, 2019, which claims the benefit of U.S. Provisional Patent Application No. 62/673,001, filed May 17, 2018, the contents of each of which are incorporated by reference in its entirety.

### INCORPORATION BY REFERENCE OF MATERIAL SUBMITTED ELECTRONICALLY

A sequence listing, which is a part of the present disclosure, is submitted concurrently with the specification. The name of the file containing the sequence listing is “50019B_SubSeqlisting.xml”. The sequence listing was created on May 7, 2025, and is 315,392 bytes in size. The subject matter of the sequence listing is incorporated by reference herein in its entirety.

### FIELD OF THE INVENTION

The present disclosure relates to methods of strain optimization to enhance secretion of proteins or metabolites from cells. The present disclosure also relates to compositions resulting from those methods. In particular, the disclosure relates to yeast cells selected or genetically engineered to enhance secretion of recombinant proteins expressed by the yeast cells, while minimizing or improving growth yields, and to methods of cultivating yeast cells for the production of useful compounds.

### BACKGROUND OF THE INVENTION

The methylotrophic yeast Pichia pastoris is widely used in the production of recombinant proteins. P. pastoris grows to high cell density, provides tightly controlled methanol-inducible trans gene expression and is capable of secreting recombinant proteins into a defined media.

However, much of the expressed recombinant protein remains located intracellularly, which can make collection difficult and can negatively impact cell growth and longevity. Furthermore, recombinantly expressed proteins may be degraded in the cell before they are secreted, resulting in a mixture of proteins that includes fragments of recombinantly expressed proteins and a decreased yield of full-length recombinant proteins. What is needed, therefore, are tools and engineered strains to enhance secretion in P. pastoris, while mitigating any loss of or improving recombinant protein productivity, including maintaining growth characteristics, as many modifications can negatively impact the natural functioning of the cell.

What is needed, therefore, are modified organisms and methods of using these organisms to produce and secrete recombinant proteins while minimizing any negative effect on cell growth and productivity.

### SUMMARY OF THE INVENTION

In some embodiments, provided herein is A Pichia pastoris microorganism, in which the activity of SEC72 has been eliminated or the sec72 gene has been deleted, and wherein said microorganism expresses a recombinant protein. In some embodiments, the microorganism further compress a recombinantly expressed SSH1 translocon complex.

In some embodiments, the SEC72 comprises a polypeptide sequence at least 95% identical to SEQ ID NO: 1. In some embodiments, the SEC72 comprises SEQ ID NO: 1. In some embodiments, the SEC72 is encoded by a sec72 gene. In some embodiments, the sec72 gene comprises a polynucleotide sequence at least 95% identical to SEQ ID NO: 2. In some embodiments, the sec72 gene comprises at least 15, 20, 25, 30, 40, or 50 contiguous nucleotides of SEQ ID NO: 2. In some embodiments, the sec72 gene comprises SEQ ID NO: 2. In some embodiments, the sec72 gene is at locus PAS_chr2-1_0448 of said microorganism.

In some embodiments, the SSH1 translocon complex comprises a first polypeptide sequence at least 95% identical to SEQ ID NO: 4, a second polypeptide sequence at least 95% identical to SEQ ID NO: 6, and a third polypeptide sequence at least 95% identical to SEQ ID NO: 8. In some embodiments, the SSH1 translocon complex comprises a first polypeptide comprising SEQ ID NO: 4, a second polypeptide comprising SEQ ID NO: 6, and a third polypeptide comprising SEQ ID NO: 8.

In some embodiments, the microorganism further comprises a recombinantly expressed translocon complex. In some embodiments, the translocon complex is expressed from a recombinant SSH1 gene, a recombinant SSS1 gene, and a recombinant SBH2 gene.

In some embodiments, the SSH1 gene comprises SEQ ID NO: 3. In some embodiments, the SSH1 gene comprises a polynucleotide sequence at least 95% identical to SEQ ID NO: 3. In some embodiments, the SSH1 gene comprises at least 15, 20, 25, 30, 40, or 50 contiguous nucleotides of SEQ ID NO: 3.

In some embodiments, the SSS1 gene comprises SEQ ID NO: 5. In some embodiments, the SSS1 gene comprises a polynucleotide sequence at least 95% identical to SEQ ID NO: 5. In some embodiments, the SSS1 gene comprises at least 15, 20, 25, 30, 40, or 50 contiguous nucleotides of SEQ ID NO: 5.

In some embodiments, the SBH2 gene comprises SEQ ID NO: 7. In some embodiments, the SBH2 gene comprises a polynucleotide sequence at least 95% identical to SEQ ID NO: 7. In some embodiments, the SBH2 gene comprises at least 15, 20, 25, 30, 40, or 50 contiguous nucleotides of SEQ ID NO: 7.

In some embodiments, expression of said recombinant SSH1 gene to increase levels of said SSH1 translocon in said microorganism above that expressed by the native organism improves the growth rate and/or fermentation performance of said microorganism.

In some embodiments, the translocon complex comprises an SSH1 protein, an SSS1 protein, and an SBH2 protein. In some embodiments, the SSH1 protein comprises a polypeptide sequence at least 95% identical to SEQ ID NO: 4. In some embodiments, the SSH1 protein comprises SEQ ID NO: 4. In some embodiments, the SSS1 protein comprises a polypeptide sequence at least 95% identical to SEQ ID NO: 6. In some embodiments, the SSS1 protein comprises SEQ ID NO: 6. In some embodiments, the SBH2 protein comprises a polypeptide sequence at least 95% identical to SEQ ID NO: 8. In some embodiments, the SBH2 protein comprises SEQ ID NO: 8.

In some embodiments, the activity of a YPS1-1 protease and a YPS1-2 protease in said microorganism has been attenuated or eliminated.

In some embodiments, the YPS1-1 protease comprises a polypeptide sequence at least 95% identical to SEQ ID NO: 10. In some embodiments, the YPS1-1 protease comprises SEQ ID NO: 10. In some embodiments, the YPS1-1 protease is encoded by a YPS1-1 gene. In some embodiments, the YPS1-1 gene comprises a polynucleotide sequence at least 95% identical to SEQ ID NO: 9. In some embodiments, the YPS1-1 gene comprises at least 15, 20, 25, 30, 40, or 50 contiguous nucleotides of SEQ ID NO: 9. In some embodiments, the YPS1-1 gene comprises SEQ ID NO: 9. In some embodiments, the YPS1-1 gene is at locus PAS_chr4_0584 of said microorganism.

In some embodiments, the YPS1-2 protease comprises a polypeptide sequence at least 95% identical to SEQ ID NO: 12. In some embodiments, the YPS1-2 protease comprises SEQ ID NO: 12. In some embodiments, the YPS1-2 protease is encoded by a YPS1-2 gene. In some embodiments, the YPS1-2 gene comprises a polynucleotide sequence at least 95% identical to SEQ ID NO: 11. In some embodiments, the YPS1-2 gene comprises at least 15, 20, 25, 30, 40, or 50 contiguous nucleotides of SEQ ID NO: 11. In some embodiments, the YPS1-2 gene comprises SEQ ID NO: 11. In some embodiments, the YPS1-2 gene is at locus PAS_chr3_1157 of said microorganism.

In some embodiments, the YPS1-1 gene or said YPS1-2 gene, or both, of said microorganism has been mutated or knocked out. In some embodiments, the activity of one or more additional proteases of said microorganism has been attenuated or eliminated.

In some embodiments, the recombinant protein expressed by said microorganism comprises at least one block polypeptide sequence from a silk protein. In some embodiments, the recombinant protein comprises a silk-like polypeptide. In some embodiments, the silk-like polypeptide comprises one or more repeat sequences {GGY-[GPG-X₁]ₙ₁-GPS-(A)ₙ₂}ₙ₃, wherein X1=SGGQQ or GAGQQ or GQGPY or AGQQ or SQ; n1 is from 4 to 8; n2 is from 6 to 20; and n3 is from 2 to 20. In some embodiments, the silk-like polypeptide comprises comprises a polypeptide sequence encoded by SEQ ID NO: 21.

In some embodiments, the recombinant protein comprises a secretion signal peptide. In some embodiments, the secretion signal peptide is selected from the group consisting of: a PEP4 signal sequence, a CPY+4 signal sequence, a DAP2 signal sequence, and a MFa1 signal sequence. In some embodiments, the secretion signal peptide is selected from the group consisting of: SEQ ID NO: 84, SEQ ID NO: 86, SEQ ID NO: 88, and SEQ ID NO: 90.

Also provided herein, according to some embodiments, is a Pichia pastoris microorganism, wherein the activity of SEC72 of said microorganism has been eliminated or an sec72 gene comprising SEQ ID NO: 1 has been knocked out, wherein the microorganism comprises a recombinantly expressed SSH1 gene comprising SEQ ID NO: 3, a recombinantly expressed SSS1 gene comprising SEQ ID NO: 5, and a recombinantly expressed SBH2 gene comprising SEQ ID NO: 7, and wherein said microorganism comprises a silk-like polypeptide comprising a polypeptide sequence encoded by SEQ ID NO: 21.

Also provided herein, according to some embodiments, is a cell culture comprising a recombinant microorganism described herein.

Also provided, herein, according to some embodiments, is a cell culture comprising a recombinant microorganism as described herein, wherein said cell culture has an improved strain growth rate and fermentation performance under standard cell culture conditions as compared to a cell culture that does not comprise a recombinantly expressed SSH1 translocon complex.

Also provided, herein, according to some embodiments, is a cell culture comprising a recombinant microorganism as described herein, wherein said cell culture has an improved yield or specific productivity of said recombinant protein under standard cell culture conditions as compared to a cell culture of otherwise identical microorgansims that comprises a functional sec72 gene and does not comprise a recombinant/y expressed SSH1 translocon complex, wherein each microorganism has the same number of copies of recombinant silk polypeptide genes.

Also provided herein, according to some embodiments, is a method of producing a recombinant protein, the method comprising: culturing the recombinant microorganism described herein in a culture medium under conditions suitable for expression of the recombinantly expressed protein; and isolating the recombinant protein from the microorganism or the culture medium.

In some embodiments, the recombinant protein is secreted from said microorganism, and isolating said recombinant protein comprises collecting a culture medium comprising said secreted recombinant protein.

In some embodiments, the microorganism has an increased yield or specific productivity of said recombinant protein as compared to an otherwise identical microorganism wherein said sec72 gene is not deleted.

In some embodiments, the microorganism has an increased yield or specific productivity of said recombinant protein as compared to an otherwise identical microorganism not comprising said recombinantly expressed SSH1 translocon complex, and wherein said sec72 gene is not deleted.

Also provided herein, according to some embodiments, is a method of modifying Pichia pastoris to improve the secretion of a recombinantly expressed protein, said method comprising knocking out a gene encoding an SEC72 protein. In some embodiments, the method of modifying Pichia pastoris to improve the secretion of a recombinantly expressed protein further comprises transforming said Pichia pastoris with a vector comprising genes encoding a recombinantly expressed SSH1 translocon complex.

In some embodiments, the recombinantly expressed protein comprises a silk-like polypeptide. In some embodiments, the silk-like polypeptide comprises one or more repeat sequences {GGY-[GPG-X₁]ₙ₁-GPS-(A)ₙ₂}ₙ₃, wherein X₁=SGGQQ or GAGQQ or GQGPY or AGQQ or SQ; n1 is from 4 to 8; n2 is from 6 to 20; and n3 is from 2 to 20. In some embodiments, the recombinantly expressed protein comprises a polypeptide sequence encoded by SEQ ID NO: 21.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1 depicts groupings of genes in secretion complexes SEC, SEC61, SEC63, and SSH1. Also shown is a value of the homology between S. cerevisiae and P. pastoris translocon complex proteins.

FIG. 2 is a plasmid map of a vector for SEC72 deletion comprising a yeast selection marker flanked by SEC72 homology arms.

FIG. 3 is a plasmid map of a vector for overexpression of genes encoding proteins comprising the SSH1 complex. It includes a promoter, an open reading frame (ORF), and a terminator for SBH2, SSS1, and SSH1, as well as a yeast selection marker.

FIG. 4A is a plasmid map of a vector comprising a nourseothricin marker used with homology arms for targeted protease gene deletion. FIG. 4B and FIG. 4C are cassettes for protease knockout with homology arms targeting the desired protease gene flanking a nourseothricin resistance marker.

FIG. 5 is a plasmid map of a vector comprising an open reading frame (ORF) for expressing a silk-like polypeptide comprising a secretion signal and a FLAG tag. The ORF is operably linked to a pGCW14 promoter and a tAOX1 terminator. The vector also comprises selection markers to select successfully transformed cells.

FIG. 6 shows secretion of recombinant 18B silk-like polypeptide expressed by wild-type (WT) and Δsec72 (Δ) strains expressing 2×, 4×, or 6× copies of 18B, as measured by an enzyme-linked immunosorbent assay (i.e., ELISA).

FIG. 7 shows shows secretion of recombinant protein comprising PEP4 (sc), CPY+4 (sc), DAP2 (sc), or MFa1 (sc) signal peptides as expressed by wild-type (WT) and Δsec72 (4) strains and measured by ELISA. (sc) indicates the signal peptide derives from S. cerevisiae.

FIG. 8 shows secretion of long silk polypeptides Argiope bruennichi Major ampullate spidron 2 (i.e., Ab MaSp2) (106 kDa), and Latrodectus hesperus Major ampullate spidron 1 (i.e., Lh MaSp1 (55 kDa)) as expressed by wild-type (WT) and Δsec72 (A) strains and measured by ELISA.

FIG. 9A shows secretion of of recombinant 18B silk-like polypeptide expressed by wild-type (WT), Δsec72 (4) strains, strains overexpressing sec72 under the control of pTHI11, and strains where SEC72 was knocked down by DAmP (Decreased Abundance by mRNA Perturbation), as measured by ELISA. FIG. 9B shows secretion of of recombinant 18B silk-like polypeptide expressed by wild-type (WT), strains overexpressing non-SEC72 proteins from the SEC63 complex (i.e., sec62, sec63 and sec66) and strains overexpressing sec72, as measured by ELISA.

FIG. 10A shows reference-normalized expression in Δsec72 strains as a fold-change from the WT strain. Shading indicates range and center bar represents median among n=3 biological replicates (except n=2 for Δsec72). FIG. 10B shows a projection of centered, log-scaled, reference normalized expression data onto principal component space, left=PC1 vs. 3, right=PC2 vs. 3. Replicates are colored by strain genotype, showing low within-genotype variance compared variance across genotypes.

FIG. 11 shows growth rates observed for WT and Δsec72 strains, and comparison of each with growth rates for strains modified to overexpress the SSH1 translocon complex or SEC61.

FIG. 12A shows the production of 18B as a function of glucose (yield) for wild-type (WT) strains expressing 4 (4×) or 6 (4+2×) copies of 18B, as compared to a Δsec72 strain (4×, Δsec72) and a Δsec72 strain overexpressing the SSH1 translocon complex (4×, Δsec72, 2N SSH1). FIG. 12B shows the specific productivity (product-per biomass) of each strain.

### DETAILED DESCRIPTION

The details of various embodiments of the invention are set forth in the description below. Other features, objects, and advantages of the invention will be apparent from the description and the drawings, and from the claims.

### Definitions

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 75%, 80%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

The nucleic acids (also referred to as polynucleotides) of this present invention may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

The term “deletion” as used herein refers to the removal of one or more nucleotides from a nucleic acid molecule or one or more amino acids from a protein, the regions on either side being joined together.

The term “knock-out” as used herein is intended to refer to a gene whose level of expression or activity has been reduced to zero. In some examples, a gene is knocked-out via deletion of some or all of its coding sequence. In other examples, a gene is knocked-out via introduction of one or more nucleotides into its open reading frame, which results in translation of a non-sense or otherwise non-functional protein product.

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “regulatory element” refers to any element which affects transcription or translation of a nucleic acid molecule. These include, by way of example but not limitation: regulatory proteins (e.g., transcription factors), chaperones, signaling proteins, RNAi molecules, antisense RNA molecules, microRNAs and RNA aptamers. Regulatory elements may be endogenous to the host organism. Regulatory elements may also be exogenous to the host organism. Regulatory elements may be synthetically generated regulatory elements.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription. Promoters may be endogenous to the host organism. Promoters may also be exogenous to the host organism. Promoters may be synthetically generated regulatory elements.

Promoters useful for expressing the recombinant genes described herein include both constitutive and inducible/repressible promoters. Where multiple recombinant genes are expressed in an engineered organism of the invention, the different genes can be controlled by different promoters or by identical promoters in separate operons, or the expression of two or more genes may be controlled by a single promoter as part of an operon.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “wild-type” (i.e., “WT”) as used herein refers to a comparative strain lacking the modification being discussed. It does not refer to the native, unmodified strain, but rather to a strain lacking a selected modification. For example, when comparing two versions of a recombinant strain modified to express a recombinant silk polypeptide, where one is a sec72 KO, the KO strain may be referred to as the Δsec72 strain, while the other version is referred to as “WT.”

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology-A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as a-, a-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

Throughout this specification and claims, the word “comprise” or variations such as “comprises” or “comprising,” will be understood to imply the inclusion of a stated integer or group of integers but not the exclusion of any other integer or group of integers.

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Overview

Provided herein are recombinant strains and methods of improving secretion and productivity of recombinantly expressed proteins in yeast strains (e.g., P. pastoris).

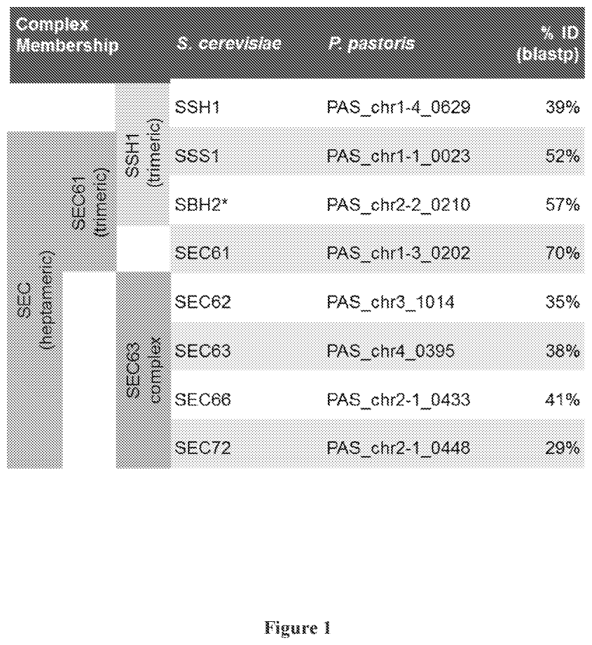

Proteins destined for secretion must first cross the membrane of the endoplasmic reticulum (ER translocation). Multiple targeting pathways recruit elongating ribosomes or fully translated proteins to the ER membrane, which polypeptide chains enter via pore-forming protein complexes called translocons. Yeasts including S. cerevisiae and P. pastoris express two translocons, the SEC61 and SSH1 complexes. Each consists of a core trimeric complex, but the SEC61 translocon also associates with the tetrameric SEC63 complex. FIG. 1 shows the protein subunits contained within each of these translocon complexes.

SEC72 encodes a nonessential member of the SEC63 complex, but deletion of SEC72 (Δsec72) results in the accumulation of some secretory precursors. Surprisingly, herein we show that Δsec72 strains have improved secretion of recombinant silk polypeptides.

In some embodiments, the strains are modified to delete the sec72 gene (SEQ ID NO: 1). As we describe and shown herein, the deletion of sec72, which expresses SEC72 (SEQ ID NO: 2), an accessory factor for translocating proteins into the ER for secretion, unexpectedly assists silk secretion in bench-scale block model assays (up to +75%). This remained true across expression levels, signal sequences, and different silks.

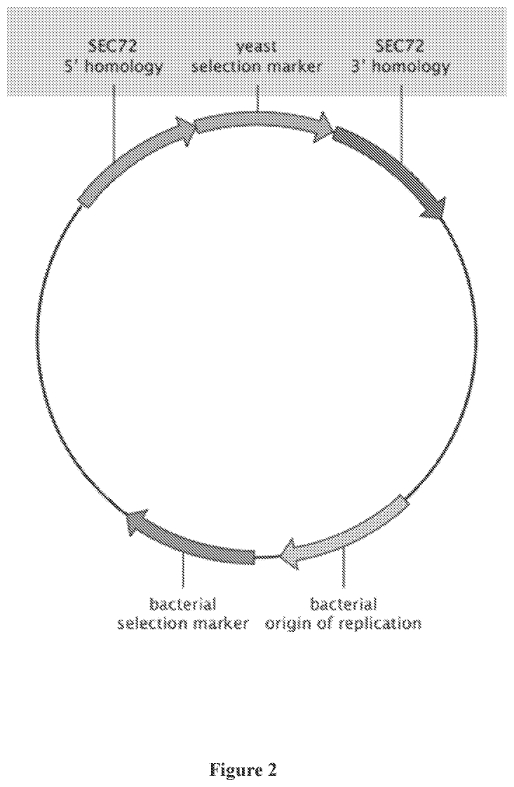

In some embodiments, deletion of sec72 is effected using a plasmid having a 5′ homology arm to sec72 and a 3′ homology arm to sec72 flanking a yeast selection marker, e.g., as shown in FIG. 2. The Δsec72 deletion elicited transcriptional adaptation that could indirectly assist secretion. Sequences of the sec72 gene and the SEC72 protein are provided in Table 1.

TABLE 1
sec72 open reading frame and SEC72 protein sequences

| Description | SEQ ID NO: | Sequence |
|---|---|---|
| sec72 | 1 | atgctaccattttcgtacgacgtgagctcaaagaaactgaaagtcaca |
| (PAS_chr2- | | ggtgactacgcagacttagaatatgacatacagcagctgaacaccttg |
| 1_0448) | | agcggagagatcctggccaataaagcagatgttccttctccaccaagt |
| ORF sequence | | aaggagtcgtttgacaagaaattgtcccacatggctcagaaattacac |
| (5′ to 3′) | | gagtcggctgtatccaacataaagacaggcaagtatcctgaggctatc |
| | | aaattgttgacgacgggtcttgaaatggttaacagaaggcccaagtac |
| | | gagagttttcagatgacgttgagtgaaatgacgatctttattgtcact |
| | | agagctgacgcttacatgatgaatggagactttgaaggggcattcaat |
| | | gatgcagatttactggtaacgctcctgccatccattccagataattac |
| | | attagaagaggggtagcccttttcaagatggggagatacgttgatgca |
| | | aaaaacaattttgagagaggactttcatttgacccagataatgcaaaa |
| | | ttgaagaaggagttagattttgtgctgaagaagatcgacgaggagaat |
| | | ggagagttatag |
| SEC72 | 2 | MLPFSYDVSSKKLKVTGDYADLEYDIQQLNTLSGEILANKADVPSPPS |
| (PAS_chr2- | | KESFDKKLSHMAQKLHESAVSNIKTGKYPEAIKLLTTGLEMVNRRPKY |
| 1_0448) | | ESFQMTLSEMTIFIVTRADAYMMNGDFEGAFNDADLLVTLLPSIPDNY |
| Protein | | IRRGVALFKMGRYVDAKNNFERGLSFDPDNAKLKKELDFVLKKIDEEN |
| Sequence | | GEL |

In some observed cases, the sec72 deletion slowed strain growth, and fermentation production screens of Δsec72 strains struggled with glucose accumulation. Thus, also provided herein, according to some embodiments, are strains recombinantly overexpressing proteins that comprise the SSH1 translocon complex. As shown herein, overexpression of the SSH1 translocon complex in Δsec72 strains improved the strain growth rate and fermentation performance, while maintaining improved secretion. Runs of this combined deletion-overexpression strain with only 4 copies of of recombinant silk-like block polypeptide expressing genes (i.e., 18B) showed similar titers and improved specific productivity (+18%) over a strain with 6 copies of silk-like block polyeptide expressing genes (i.e., 18B).

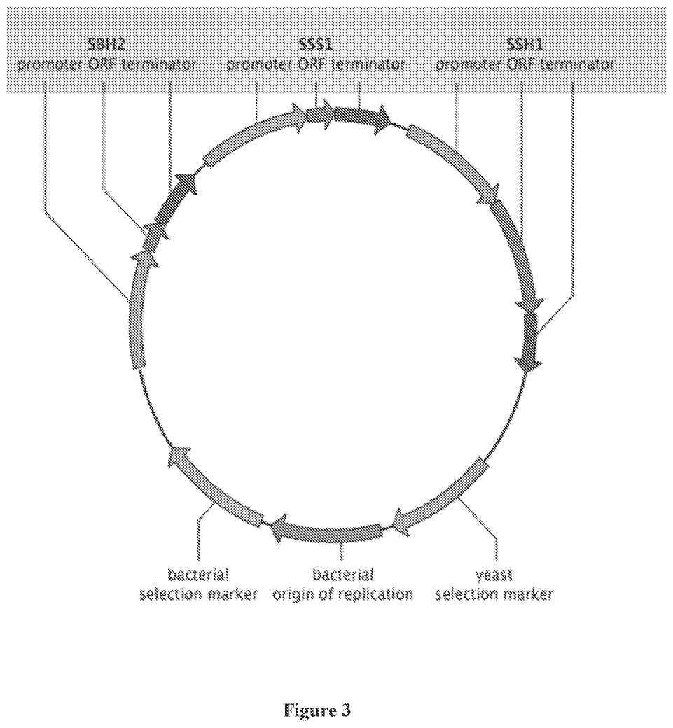

In some embodiments, the SSH1 translocon complex is overexpressed in host cells by inserting a plasmid comprising genes encoding recombinant SBH2, SSS1, and SSH1 (FIG. 3). Sequences for these genes and the expressed protein can be found in Table 2.

TABLE 2
Open reading frame and protein sequences for subunits
of the SSH1 translocon complex.

| Description | SEQ ID NO: | Sequence |
|---|---|---|
| SSH1 | 3 | atggcggggttgcgttttttagacattgcaagaccatttgtcagctgg |
| (PAS_chr1-4_0629) | | atcccggaagttgaacttccttatgaaaactgggggttcgatgaaaag |
| ORF sequence | | ctgatttactcatttttcactgctgccatctatttgattctgtccctg |
| | | cctatatacggtgtcaaatcctctgaagtcgtggacccagttccccat |
| | | ttgcgttctgccttagggagtgagaagggaacattgctggagcttggg |
| | | ttactgcctgtgattacttcggcatttatcttgcagttgttggctggt |
| | | tggaaagttttcaaagtaaactttgatctggttagtgacagaatattg |
| | | ttccaaactttgcaaaagatcacttcagtcgttatcagcatcgtatat |
| | | gctgttcttctcacattttgtgactactttactccaggtgtgtccact |
| | | gataacgtcttgtggtcccaatttctgatcatcttacagatagtggtg |
| | | gtcaacttcttggttactctactcgttgaagtcattgacaaggattac |
| | | ggattttcttcaggagctctattgttgcttgcggtttattccgccacc |
| | | aacttcgtttttggcacgattggtcttagcaccgtcaacacctccaga |
| | | tcgaacgaatctattggtgctctgattcaattattccgcaatttgagc |
| | | tctaaaccaattggtgttgccatatatgactccttcttcagagtaaac |
| | | cttcctaacttgactcaattttatctggggattgccattatttgtgtt |
| | | tgtctgttcttgaataatgcaagatacgaagtaccaattaagccaaac |
| | | aaggttcgtgccatggcctcagcttacccaatcaagctacttttcaat |
| | | ggttctttgccacttctgtacacgtggactgtgctgtacaacttgaac |
| | | cttattggtttctttgtcttcaagcttaccaacttttctcttttaggg |
| | | aacttcaaagtggacccattcggcaacaactactacgaaattacatct |
| | | ggactgctgtatttattgactcctactttcaacgctgaagctggactt |
| | | ttacccaatgttgctaagccatttgttttcattgccttctatgttggt |
| | | gttagcactttctttgctagatcgtggtccaacattaacgggtcgtca |
| | | ggcaaggacattgccaagtttttcaaggctcaaggaatctcattgtta |
| | | ggaaaaagagatgcctctgtgtctaaagagtttaacaccctagttcct |
| | | gttgcttctgcctctggagctttcctattgtcttttccagttgccgtc |
| | | gctgagttattgggtggctctggtgttccaacctctatcggaatcggt |
| | | cttttgagtggtttggctattttggaaactgttttgcaagaatggcaa |
| | | cagtctggaggtgcctcacagttctcccaatacttccagacttcttag |
| SSH1 | 4 | MAGLRFLDIARPFVSWIPEVELPYENWGFDEKLIYSFFTAAIYLILSL |
| (PAS_chr1-4_0629) | | PIYGVKSSEVVDPVPHLRSALGSEKGTLLELGLLPVITSAFILQLLAG |
| Protein Sequence | | WKVFKVNFDLVSDRILFQTLQKITSVVISIVYAVLLTFCDYFTPGVST |
| | | DNVLWSQFLIILQIVVVNFLVTLLVEVIDKDYGFSSGALLLLAVYSAT |
| | | NFVFGTIGLSTVNTSRSNESIGALIQLFRNLSSKPIGVAIYDSFFRVN |
| | | LPNLTQFYLGIAIICVCLFLNNARYEVPIKPNKVRAMASAYPIKLLFN |
| | | GSLPLLYTWTVLYNLNLIGFFVFKLTNFSLLGNFKVDPFGNNYYEITS |
| | | GLLYLLTPTFNAEAGLLPNVAKPFVFIAFYVGVSTFFARSWSNINGSS |
| | | GKDIAKFFKAQGISLLGKRDASVSKEFNTLVPVASASGAFLLSFPVAV |
| | | AELLGGSGVPTSIGIGLLSGLAILETVLQEWQQSGGASQFSQYFQTS |
| SSS1 | 5 | atgtcccaaaaagtcaccgacgtccctctggaatttgttaaggaaggt |
| (PAS_chr1-1_0023) | | tccaaattcatctctaaatgtactaaaccctctcagaaggagtactta |
| ORF sequence | | aagatagtaagagctgttggagttgggtttttaatgatgggcgtggtt |
| | | ggttacgttgtcaagctcattcatattccaatcagatatttgattgtt |
| | | taa |
| SSS1 | 6 | MSQKVTDVPLEFVKEGSKFISKCTKPSQKEYLKIVRAVGVGFLMMGVV |
| (PAS_chr1-1_0023) | | GYVVKLIHIPIRYLIV |
| Protein Sequence | | |
| SBH2 | 7 | atggtaagtgtccagtttgatgagtgcagaatggttccaagttttaga |
| (PAS_chr2-2_0210) | | ccagttactaatatttaaagtctacagcaattccaggaggacagagaa |
| ORF sequence | | cgttagctaaaagaagagcagcaaacttggataagaaacaggatgaac |
| | | caacctccgccagatctgccggtgctggaggttcttcgtctaccatgc |
| | | taaagttgtacacagacgaggcccaaggtttgaaagttgatcctttaa |
| | | ttgttcttgttcttgctgttggtttcattttcagtgtcattggtttgc |
| | | acgttgttgctaagctgacaggaaagttgatcaactaa |
| SBH2 | 8 | MSTAIPGGQRTLAKRRAANLDKKQDEPTSARSAGAGGSSSTMLKLYTD |
| (PAS_chr2-2_0210) | | EAQGLKVDPLIVLVLAVGFIFSVIGLHVVAKLTGKLIN |
| Protein Sequence | | |

### Protease Knock-Outs

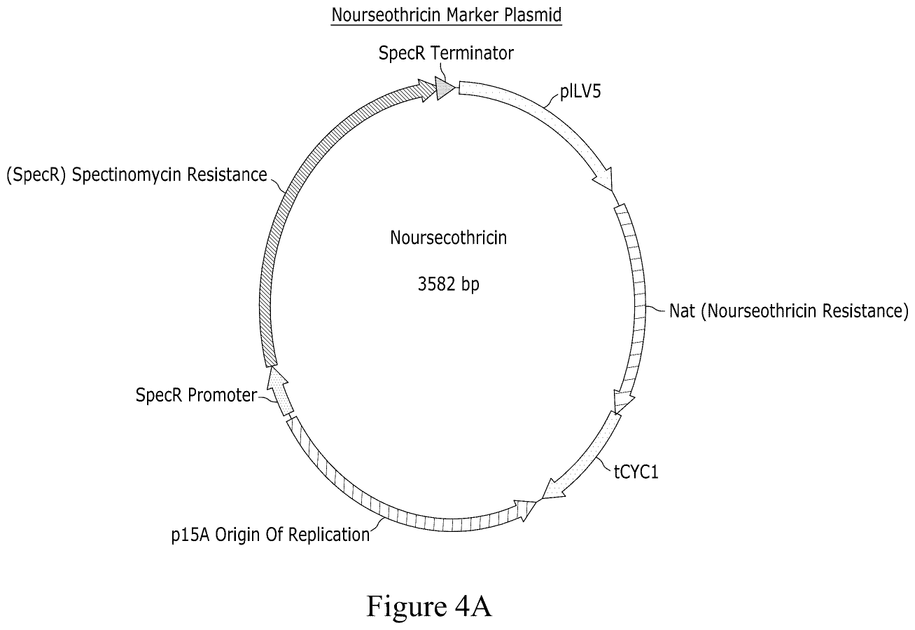

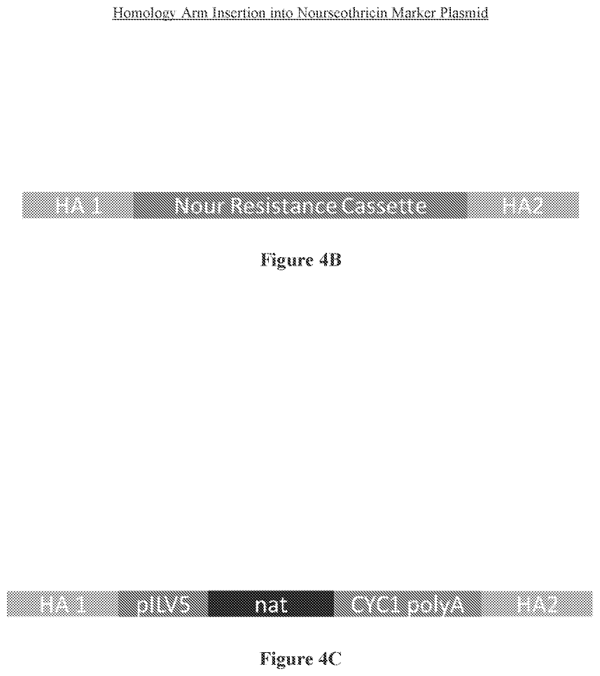

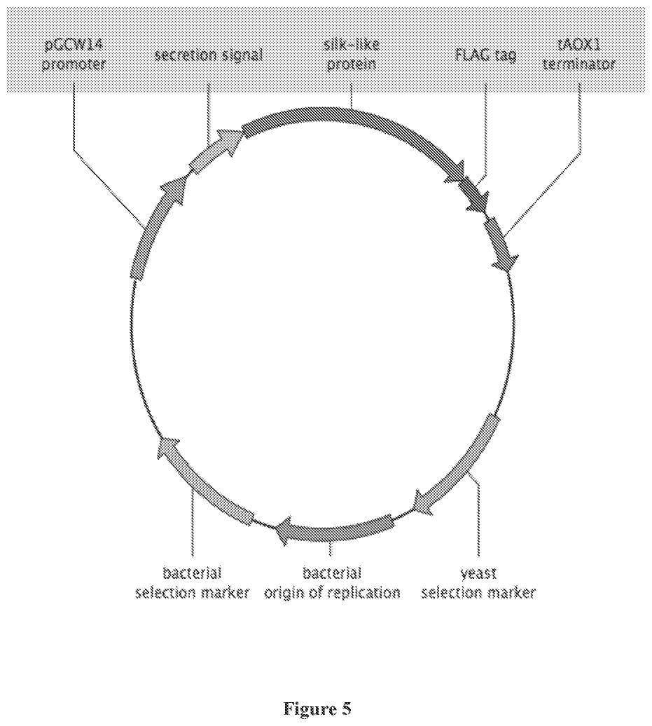

In some embodiments, to attenuate a protease activity in Pichia pastoris, the genes encoding these enzymes are inactivated or mutated to reduce or eliminate activity. This can be done through mutations or insertions into the gene itself of through modification of a gene regulatory element. This can be achieved through standard yeast genetics techniques. Examples of such techniques include gene replacement through double homologous recombination, in which homologous regions flanking the gene to be inactivated are cloned in a vector flanking a selectable maker gene (such as an antibiotic resistance gene or a gene complementing an auxotrophy of the yeast strain). In some embodiments, the Nourseothricin selection plasmid shown in FIG. 4A can be used as a base plasmid, and homology arms (HA) flanking resistance cassettes (FIG. 4B and FIG. 4C) can be used, where the homology arms specifically target the desired protease to be knocked out. Description of protease knock-outs and methods of modifying host cells to inhibit recombinant protein degradation are provided in U.S. application Ser. No. 15/724,196 and PCT Application No. PCT/US2017/054997, each of which are incorporated herein by reference in its entirety.

Alternatively, the homologous regions can be PCR-amplified and linked through overlapping PCR to the selectable marker gene. Subsequently, such DNA fragments are transformed into Pichia pastoris through methods known in the art, e.g., electroporation. Transformants that then grow under selective conditions are analyzed for the gene disruption event through standard techniques, e.g. PCR on genomic DNA or Southern blot. In an alternative experiment, gene inactivation can be achieved through single homologous recombination, in which case, e.g. the 5′ end of the gene's ORF is cloned on a promoterless vector also containing a selectable marker gene. Upon linearization of such vector through digestion with a restriction enzyme only cutting the vector in the target-gene homologous fragment, such vector is transformed into Pichia pastoris. Integration at the target gene site is confirmed through PCR on genomic DNA or Southern blot. In this way, a duplication of the gene fragment cloned on the vector is achieved in the genome, resulting in two copies of the target gene locus: a first copy in which the ORF is incomplete, thus resulting in the expression (if at all) of a shortened, inactive protein, and a second copy which has no promoter to drive transcription.

Alternatively, transposon mutagenesis is used to inactivate the target gene. A library of such mutants can be screened through PCR for insertion events in the target gene.

The functional phenotype (i.e., deficiencies) of an engineered/knockout strain can be assessed using techniques known in the art. For example, a deficiency of an engineered strain in protease activity can be ascertained using any of a variety of methods known in the art, such as an assay of hydrolytic activity of chromogenic protease substrates, band shifts of substrate proteins for the selected protease, among others.

Attenuation of a protease activity described herein can be achieved through mechanisms other than a knockout mutation. For example, a desired protease can be attenuated via amino acid sequence changes by altering the nucleic acid sequence, placing the gene under the control of a less active promoter, down-regulation, expressing interfering RNA, ribozymes or antisense sequences that target the gene of interest, or through any other technique known in the art. In preferred strains, the protease activity of proteases encoded at PAS_chr4_0584 (YPS1-1) and PAS_chr3_1157 (YPS1-2) (e.g., polypeptides comprising SEQ ID NO: 10 and 12) is attenuated by any of the methods described above. In some aspects, the invention is directed to methylotrophic yeast strains, especially Pichia pastoris strains, wherein a YPS1-1 and a YPS1-2 gene (e.g., as set forth in SEQ ID NO: 9 and SEQ ID NO: 11) have been inactivated. In some embodiments, additional protease encoding genes may also be knocked-out in accordance with the methods provided herein to further reduce protease activity of a desired protein product expressed by the strain.

TABLE 3
Open reading frame nucleotide sequence and polypeptide sequence for
proteases targeted for deletion in P. pastoris

| Description | SEQ ID NO: | Sequence |
|---|---|---|
| PAS_chr4_0584 | 9 | atgttgaaggatcagttcttgttatgggttgctttgatagcgagcgtaccggtttc |
| (YPS1-1) | | cggcgtgatggcagctcctagcgagtccgggcataacacggttgaaaaacgagatg |
| ORF sequence | | ccaaaaacgttgttggcgttcaacagttggacttcagcgttctgaggggtgattcc |
| (5′ to 3′) | | ttcgaaagtgcctcttcagagaacgtgcctcggcttgtgaggagagatgacacgct |
| | | agaagctgagctaatcaaccagcaatcattctacttgtcacgactgaaagttggat |
| | | cacatcaagcggatattggaatcctagtggacacaggatcctctgatttatgggta |
| | | atggactcggtaaacccatactgcagtagccgttcccgcgtgaagagagatataca |
| | | cgatgagaagatcgccgaatgggatcccatcaatctcaagaaaaatgaaacttctc |
| | | agaataaaaatttttgggattggctcgttggaactagcactagttctccttccacc |
| | | gccacggcaactggtagtggtagtggtagtggtagtggtagtggtagtggtagtgc |
| | | tgccacagccgtatcggtaagttctgcacaggcaacattggattgctctacgtatg |
| | | gaacgtttgatcacgctgattcctcgacgttccatgacaataatacagactttttc |
| | | atctcatacgctgataccacttttgcttcaggaatctggggttatgacgacgtcat |
| | | tatcgacggcatagaggtgaaagaactttccttcgccgttgcagacatgaccaatt |
| | | cctctattggtgtgttaggtattggactgaaaggcctagaatccacatatgctagt |
| | | gcatcttcggtcagtgaaatgtatcagtatgacaatttgccagccaagatggtcac |
| | | cgatgggttgatcaacaaaaatgcatactccttgtacttgaactccaaggacgcct |
| | | caagtggttccatcctctttggaggtgtggatcatgaaaaatattcgggacaattg |
| | | ttgacagttccagtcatcaacacactcgcttccagtggttacagagaggcaattcg |
| | | tttacaaattactttaaatggaatagatgtgaaaaagggttctgaccagggaactc |
| | | ttttacaagggagatttgctgcattattggactctggagctacgctaacgtatgct |
| | | ccttcttctgttttaaattcaattggccggaacctgggcggctcctatgattcgtc |
| | | aagacaagcttataccattcgttgtgtttctgcatcagataccacttctctggtat |
| | | tcaattttgggggtgctacagtggaagtttccctgtacgatctacagattgcaaca |
| | | tattacaccgggggaagtgccacgcaatgtcttattggaatattcagctctggaag |
| | | tgatgagtttgtgctcggtgataccttcttgaggtcagcctacgtggtttacgatc |
| | | ttgatgggcttgaagtgtcgcttgcccaagccaacttcaacgaaaccgattctgat |
| | | gttgaggctattacctccagtgtaccttccgctactcgtgcatccggatacagttc |
| | | tacatggtctggttctgccagcggtacagtttacacttcggttcagatggaatccg |
| | | gtgctgcttccagctccaactcttctggatcgaatatgggttcctcttcctcatcg |
| | | tcctcttcatcgtcctcgacttccagtggagacgaagaaggagggagctccgccaa |
| | | cagggtccccttcagctacctttctctctgtttggtagttattctcggcgtgtgta |
| | | tagtatag |
| PAS_chr4_0584 | 10 | MLKDQFLLWV ALIASVPVSG VMAAPSESGH NTVEKRDAKN VVGVQQLDFS |
| (YPS1-4) | | VLRGDSFESA SSENVPRLVR RDDTLEAELI NQQSFYLSRL KVGSHQADIG |
| Polypeptide | | ILVDTGSSDL WVMDSVNPYC SSRSRVKRDI HDEKIAEWDP INLKKNETSQ |
| Sequence | | NKNFWDWLVG TSTSSPSTAT ATGSGSGSGS GSGSGSAATA VSVSSAQATL |
| | | DCSTYGTFDH ADSSTFHDNN TDFFISYADT TFASGIWGYD DVIIDGIEVK |
| | | ELSFAVADMT NSSIGVLGIG LKGLESTYAS ASSVSEMYQY DNLPAKMVTD |
| | | GLINKNAYSL YLNSKDASSG SILFGGVDHE KYSGQLLTVP VINTLASSGY |
| | | REAIRLQITL NGIDVKKGSD QGTLLQGRFA ALLDSGATLT YAPSSVLNSI |
| | | GRNLGGSYDS SRQAYTIRCV SASDTTSLVF NFGGATVEVS LYDLQIATYY |
| | | TGGSATQCLI GIFSSGSDEF VLGDTFLRSA YVVYDLDGLE VSLAQANFNE |
| | | TDSDVEAITS SVPSATRASG YSSTWSGSAS GTVYTSVQME SGAASSSNSS |
| | | GSNMGSSSSS SSSSSSTSSG DEEGGSSANR VPFSYLSLCL VVILGVCIV |
| PAS_chr3_1157 | 11 | atgatcatcaaccacttggtattgacagccctcagcattgcactagcaagtgcgca |
| (YPS1-2) | | actccaatcgcctttcaaggctaacaagttgccattcaaaaagtttatcattccaa |
| ORF sequence | | cgacccaaaggaccgtttaattaagagagatgactacgagtccctcgacttgagac |
| (5′ to 3′) | | acatcggagtcttgtacactgcagagatccaaattggatctgacgaaactgaaatt |
| | | gaggtcattgtcgacactggttctgccgacttgtgggtcatcgattccgacgctgc |
| | | cgtctgtgagttatcctacgatgagattgaggccaatagcttttcctcggcttctg |
| | | ccaaattcatggacaagatagctcctccatcacaagagctcctggatgggctgagt |
| | | gagtttggatttgctctcgatggtgaaatttctcaatacctagccgataaatctgg |
| | | acgtgtttcgaaaagagaggaaaatcaacaagatttcaacattaaccgtgacgagc |
| | | ctgtgtgtgaacagtttggttccttcgattctagttcttccgacactttccaaagc |
| | | aacaattcagcttttggtattgcttaccttgatggaaccactgctaacggaacttg |
| | | ggtcagggacacagtccgcatcggcgactttgccatcagccaacagagttttgcct |
| | | tagtcaacatcacagataactacatgggaatcttgggtctcggtcctgctacccaa |
| | | caaaccaccaatagtaacccaattgcagcaaacagatttacttatgatggtgttgt |
| | | ggattcattgcggtcccaaggatttatcaattcagcatcgttttctgtttacttgt |
| | | ctccagatgaagataacgagcacgacgaattcagcgacggagaaattttatttggt |
| | | gctattgatagggccaagatagacgggccatttagacttttcccatatgtcaatcc |
| | | ttacaaaccagtttaccccgatcaatatacttcctacgttacagtgtccacaattg |
| | | cggtgtcttcgtcagatgaaactctcattattgaaagacgtcctcgtttggcatta |
| | | atcgatacaggtgccaccttctcctatttgccaacctacccattgattcgtttagc |
| | | gttttccatccatggaggctttgaatatgtttctcaattgggactatttgtcattc |
| | | gtacaagttctctgtctgttgctagaaataaggtgattgagttcaagtttggtgaa |
| | | gacgttgtgatccaatccccagtttctgatcatctattggacgtctcaggcctttt |
| | | tactgatggccaacaatactccgcattaactgtacgtgaaagtcttgacggacttt |
| | | ccattctaggtgatacattcatcaaatcggcctacttattctttgacaatgaaaac |
| | | agccagctgggtattggtcagatcaacgtcactgatgacgaggatattgaggtggt |
| | | cggtgatttcactattgaacgagacccagcctactcctctacttggtctagcgatt |
| | | tacctcatgaaacacccactagggctttgagtactgcttcagggggaggccttggt |
| | | accggaataaacacggccacaagtcgtgcaagttctcgttccacatctggctctac |
| | | ttcacgaacttcttctacatctggctctgcttctggtacttcttcaggtgcatctt |
| | | ctgctactcaaaatgacgaaacatccactgatcttggagctccagctgcatcttta |
| | | agtgcaacgccatgtctttttgccatcttgctgctcatgttgtag |
| PAS_chr3_1157 | 12 | MIINHLVLTA LSIALANDYE SLDLRHIGVL YTAEIQIGSD ETEIEVIVDT |
| (YPS1-2) | | GSADLWVIDS DAAVCELSYD EIEANSFSSA SAKFMDKIAP PSQELLDGLS |
| Polypeptide | | EFGFALDGEI SQYLADKSGR VSKREENQQD FNINRDEPVC EQFGSFDSSS |
| Sequence | | SDTFQSNNSA FGIAYLDGTT ANGTWVRDTV RIGDFAISQQ SFALVNITDN |
| | | YMGILGLGPA TQQTTNSNPI AANRFTYDGV VDSLRSQGFI NSASFSVYLS |
| | | PDEDNEHDEF SDGEILFGAI DRAKIDGPFR LFPYVNPYKP VYPDQYTSYV |
| | | TVSTIAVSSS DETLIIERRP RLALIDTGAT FSYLPTYPLI RLAFSIHGGF |
| | | EYVSQLGLFV IRTSSLSVAR NKVIEFKFGE DVVIQSPVSD HLLDVSGLFT |
| | | DGQQYSALTV RESLDGLSIL GDTFIKSAYL FFDNENSQLG IGQINVTDDE |
| | | DIEVVGDFTI ERDPAYSSTW SSDLPHETPT RALSTASGGG LGTGINTATS |
| | | RASSRSTSGS TSRTSSTSGS ASGTSSGASS ATQNDETSTD LGAPAASLSA |
| | | TPCLFAILLL ML |

### Production of Recombinant Strains

Provided herein are methods of transforming a strain to reduce activity, e.g., using vectors to deliver recombinant genes or to knock-out or otherwise attenuate endogenous genes as desired. These vectors can take the form of a vector backbone containing a replication origin and a selection marker (typically antibiotic resistance, although many other methods are possible), or a linear fragment that enables incorporation into the target cell's chromosome. The vectors should correspond to the organism and insertion method chosen.

Once the elements of a vector are selected, construction of the vector can be performed in many different ways. In an embodiment, a DNA synthesis service or a method to individually make every vector may be used.

Once the DNA for each vector (including the additional elements required for insertion and operation) is acquired, it must be assembled. There are many possible assembly methods including (but not limited to) restriction enzyme cloning, blunt-end ligation, and overlap assembly [see, e.g., Gibson, D. G., et al., Enzymatic assembly of DNA molecules up to several hundred kilobases. Nature methods, 6 (5), 343-345 (2009), and GeneArt Kit (tools.invitrogen.com/content/sfs/manuals/geneart_seamless_cloning_and_assembly_man.pdf)]. Overlap assembly provides a method to ensure all of the elements get assembled in the correct position and do not introduce any undesired sequences.

The vectors generated above can be inserted into target cells using standard molecular biology techniques, e.g., molecular cloning. In an embodiment, the target cells are already engineered or selected such that they already contain the genes required to make the desired product, although this may also be done during or after further vector insertion.

Depending on the organism and library element type (plasmid or genomic insertion), several known methods of inserting the vector comprising DNA to incorporate into the cells may be used. These may include, for example, transformation of microorganisms able to take up and replicate DNA from the local environment, transformation by electroporation or chemical means, transduction with a virus or phage, mating of two or more cells, or conjugation from a different cell.

Several methods are known in the art to introduce recombinant DNA in bacterial cells that include but are not limited to transformation, transduction, and electroporation, see Sambrook, et al., Molecular Cloning: A Laboratory Manual (1989), Second Edition, Cold Spring Harbor Press, Plainview, N.Y. Non-limiting examples of commercial kits and bacterial host cells for transformation include NovaBlue Singles™ (EMD Chemicals Inc., NJ, USA), Max Efficiency® DH5a™, One Shot@ BL21 (DE3) E. coli cells, One Shot® BL21 (DE3) pLys E. coli cells (Invitrogen Corp., Carlsbad, Calif., USA), XL1-Blue competent cells (Stratagene, CA, USA). Non limiting examples of commercial kits and bacterial host cells for electroporation include Zappers™ electrocompetent cells (EMD Chemicals Inc., NJ, USA), XL1-Blue Electroporation-competent cells (Stratagene, CA, USA), ElectroMAX™ A. tumefaciens LBA4404 Cells (Invitrogen Corp., Carlsbad, Calif., USA).

Several methods are known in the art to introduce recombinant nucleic acid in eukaryotic cells. Exemplary methods include transfection, electroporation, liposome mediated delivery of nucleic acid, microinjection into to the host cell, see Sambrook, et al., Molecular Cloning: A Laboratory Manual (1989), Second Edition, Cold Spring Harbor Press, Plainview, N.Y. Non-limiting examples of commercial kits and reagents for transfection of recombinant nucleic acid to eukaryotic cell include Lipofectamine™ 2000, Optifect™ Reagent, Calcium Phosphate Transfection Kit (Invitrogen Corp., Carlsbad, Calif., USA), GeneJammer® Transfection Reagent, LipoTAXI® Transfection Reagent (Stratagene, CA, USA). Alternatively, recombinant nucleic acid may be introduced into insect cells (e.g. sf9, sf21, High Five™) by using baculo viral vectors.

Transformed cells are isolated so that each clone can be tested separately. In an embodiment, this is done by spreading the culture on one or more plates of culture media containing a selective agent (or lack of one) that will ensure that only transformed cells survive and reproduce. This specific agent may be an antibiotic (if the library contains an antibiotic resistance marker), a missing metabolite (for auxotroph complementation), or other means of selection. The cells are grown into individual colonies, each of which contains a single clone.

Colonies are screened for desired production of a protein, metabolite, or other product, for reduction in protease activity, for growth, or for increase in secretion activity. In an embodiment, screening identifies recombinant cells having the highest (or high enough) product production titer or efficiency. This includes a decreased proportion of degradation products or an increased total amount of desired polypeptides secreted from a cell and collected from a cell culture.

This assay can be performed by growing individual clones, one per well, in multi-well culture plates. Once the cells have reached an appropriate biomass density, they are induced with methanol. After a period of time, typically 24-72 hours of induction, the cultures are harvested by spinning in a centrifuge to pellet the cells and removing the supernatant. The supernatant from each culture can then be tested for to determine whether desired secretion amounts have been achieved.

### Silk Sequences

In some embodiments, the modified strains described herein recombinantly express a silk-like polypeptide sequence. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful fibers by secretion from an industrially scalable microorganism. Large (approximately 40 kDa to approximately 100 kDa) block copolymer polypeptides engineered from silk repeat domain fragments, including sequences from almost all published amino acid sequences of spider silk polypeptides, can be expressed in the modified microorganisms described herein. In some embodiments, silk polypeptide sequences are matched and designed to produce highly expressed and secreted polypeptides capable of fiber formation. In some embodiments, knock-out of protease genes or reduction of protease activity in the host modified strain reduces degradation of the silk like polypeptides. In some embodiments, knock-out of sec72 and overexpression of an SSH1 translocon complex improves secretion while mitigating defects in growth, maintaining, or improving growth of the strain.

Provided herein, in several embodiments, are compositions for expression and secretion of block copolymers engineered from a combinatorial mix of silk polypeptide domains across the silk polypeptide sequence space, wherein the block copolymers have minimal degradation. In some embodiments provided herein are methods of secreting block copolymers in scalable organisms (e.g., yeast, fungi, and gram positive bacteria) with minimal degradation. In some embodiments, the block copolymer polypeptide comprises 0 or more N-terminal domains (NTD), 1 or more repeat domains (REP), and 0 or more C-terminal domains (CTD). In some aspects of the embodiment, the block copolymer polypeptide is >100 amino acids of a single polypeptide chain. In some embodiments, the block copolymer polypeptide comprises a domain that is at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of a block copolymer polypeptide as disclosed in International Publication No. WO/2015/042164, “Methods and Compositions for Synthesizing Improved Silk Fibers,” incorporated by reference in its entirety.

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011). For example:

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block copolymer polypeptides having sequence similarity and/or amino acid composition similarity to the repeat domains of native silk proteins can be used to manufacture on commercial scales consistent silk-like fibers that recapitulate the properties of corresponding natural silk fibers.

In some embodiments, a list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for expression in Pichia (Komagataella) pastoris. The DNA sequences are each cloned into an expression vector and transformed into Pichia (Komagataella) pastoris. In some embodiments, various silk domains demonstrating successful expression and secretion are subsequently assembled in combinatorial fashion to build silk molecules capable of fiber formation.

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. In some embodiments, block sequences comprise a glycine rich region followed by a polyA region. In some embodiments, short (˜1-10) amino acid motifs appear multiple times inside of blocks. For the purpose of this invention, blocks from different natural silk polypeptides can be selected without reference to circular permutation (i.e., identified blocks that are otherwise similar between silk polypeptides may not align due to circular permutation). Thus, for example, a “block” of SGAGG (SEQ ID NO: 23) is, for the purposes of the present invention, the same as GSGAG (SEQ ID NO: 24) and the same as GGSGA (SEQ ID NO: 25); they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 4
Samples of Block Sequences

| Species | Silk Type | Representative Block Amino Acid Sequence |
|---|---|---|
| Aliatypus gulosus | Fibroin 1 | GAASSSSTIITTKSASASAAADASAAATASAASRSSANAAASAFAQS |
| | | FSSILLESGYFCSIFGSSISSSYAAAIASAASRAAAESNGYTTHAYA |
| | | CAKAVASAVERVTSGADAYAYAQAISDAISHALLYTGRLNTANANSL |
| | | ASAFAYAFANAAAQASASSASAGAASASGAASASGAGSAS |
| | | (SEQ ID NO: 26) |
| Plectreurys | Fibroin 1 | GAGAGAGAGAGAGAGAGSGASTSVSTSSSSGSGAGAGAGSGAGSGAG |
| tristis | | AGSGAGAGAGAGGAGAGFGSGLGLGYGVGLSSAQAQAQAQAAAQAQA |
| | | QAQAQAYAAAQAQAQAQAQAQ |
| | | (SEQ ID NO: 27) |
| Plectreurys | Fibroin 4 | GAAQKQPSGESSVATASAAATSVTSGGAPVGKPGVPAPIFYPQGPLQ |
| tristis | | QGPAPGPSNVQPGTSQQGPIGGVGGSNAFSSSFASALSLNRGFTEVI |
| | | SSASATAVASAFQKGLAPYGTAFALSAASAAADAYNSIGSGANAFAY |
| | | AQAFARVLYPLVQQYGLSSSAKASAFASAIASSFSSGTSGQGPSIGQ |
| | | QQPPVTISAASASAGASAAAVGGGQVGQGPYGGQQQSTAASASAAAA |
| | | TATS |
| | | (SEQ ID NO: 28) |
| Araneus gemmoides | TuSp | GNVGYQLGLKVANSLGLGNAQALASSLSQAVSAVGVGASSNAYANAV |
| | | SNAVGQVLAGQGILNAANAGSLASSFASALSSSAASVASQSASQSQA |
| | | ASQSQAAASAFRQAASQSASQSDSRAGSQSSTKTTSTSTSGSQADSR |
| | | SASSSASQASASAFAQQSSASLSSSSSFSSAFSSATSISAV |
| | | (SEQ ID NO: 29) |
| Argiope aurantia | TuSp | GSLASSFASALSASAASVASSAAAQAASQSQAAASAFSRAASQSASQ |
| | | SAARSGAQSISTTTTTSTAGSQAASQSASSAASQASASSFARASSAS |
| | | LAASSSFSSAFSSANSLSALGNVGYQLGFNVANNLGIGNAAGLGNAL |
| | | SQAVSSVGVGASSSTYANAVSNAVGQFLAGQGILNAANA |
| | | (SEQ ID NO: 30) |
| Deinopis spinosa | TuSp | GASASAYASAISNAVGPYLYGLGLFNQANAASFASSFASAVSSAVAS |
| | | ASASAASSAYAQSAAAQAQAASSAFSQAAAQSAAAASAGASAGAGAS |
| | | AGAGAVAGAGAVAGAGAVAGASAAAASQAAASSSASAVASAFAQSAS |
| | | YALASSSAFAMAFASATSAGYLGSLAYQLGLTTAYNLGLSNAQAFAS |
| | | TLSQAVTGVGL |
| | | (SEQ ID NO: 31) |
| Nephila clavipes | TuSp | GATAASYGNALSTAAAQFFATAGLLNAGNASALASSFARAFSASAES |
| | | QSFAQSQAFQQASAFQQAASRSASQSAAEAGSTSSSTTTTTSAARSQ |
| | | AASQSASSSYSSAFAQAASSSLATSSALSRAFSSVSSASAASSLAYS |
| | | IGLSAARSLGIADAAGLAGVLARAAGALGQ |
| | | (SEQ ID NO: 32) |
| Argiope | Flag | GGAPGGGPGGAGPGGAGFGPGGGAGFGPGGGAGFGPGGAAGGPGGPG |
| trifasciata | | GPGGPGGAGGYGPGGAGGYGPGGVGPGGAGGYGPGGAGGYGPGGSGP |
| | | GGAGPGGAGGEGPVTVDVDVTVGPEGVGGGPGGAGPGGAGFGPGGGA |
| | | GFGPGGAPGAPGGPGGPGGPGGPGGPGGVGPGGAGGYGPGGAGGVGP |
| | | AGTGGFGPGGAGGFGPGGAGGFGPGGAGGFGPAGAGGYGPGGVGPGG |
| | | AGGFGPGGVGPGGSGPGGAGGEGPVTVDVDVSV |
| | | (SEQ ID NO: 33) |
| Nephila clavipes | Flag | GVSYGPGGAGGPYGPGGPYGPGGEGPGGAGGPYGPGGVGPGGSGPGG |
| | | YGPGGAGPGGYGPGGSGPGGYGPGGSGPGGYGPGGSGPGGYGPGGSG |
| | | PGGYGPGGYGPGGSGPGGSGPGGSGPGGYGPGGTGPGGSGPGGYGPG |
| | | GSGPGGSGPGGYGPGGSGPGGFGPGGSGPGGYGPGGSGPGGAGPGGV |
| | | GPGGFGPGGAGPGGAAPGGAGPGGAGPGGAGPGGAGPGGAGPGGAGP |
| | | GGAGGAGGAGGSGGAGGSGGTTIIEDLDITIDGADGPITISEELPIS |
| | | GAGGSGPGGAGPGGVGPGGSGPGGVGPGGSGPGGVGPGGSGPGGVGP |
| | | GGAGGPYGPGGSGPGGAGGAGGPGGAYGPGGSYGPGGSGGPGGAGGP |
| | | YGPGGEGPGGAGGPYGPGGAGGPYGPGGAGGPYGPGGEGGPYGP |
| | | (SEQ ID NO: 34) |
| Latrodectus | AcSp | GINVDSDIGSVTSLILSGSTLQMTIPAGGDDLSGGYPGGFPAGAQPS |
| hesperus | | GGAPVDFGGPSAGGDVAAKLARSLASTLASSGVFRAAFNSRVSTPVA |
| | | VQLTDALVQKIASNLGLDYATASKLRKASQAVSKVRMGSDTNAYALA |
| | | ISSALAEVLSSSGKVADANINQIAPQLASGIVLGVSTTAPQFGVDLS |
| | | SINVNLDISNVARNMQASIQGGPAPITAEGPDFGAGYPGGAPTDLSG |
| | | LDMGAPSDGSRGGDATAKLLQALVPALLKSDVFRAIYKRGTRKQVVQ |
| | | YVTNSALQQAASSLGLDASTISQLQTKATQALSSVSADSDSTAYAKA |
| | | FGLAIAQVLGTSGQVNDANVNQIGAKLATGILRGSSAVAPRLGIDLS |
| | | (SEQ ID NO: 35) |
| Argiope | AcSp | GAGYTGPSGPSTGPSGYPGPLGGGAPFGQSGFGGSAGPQGGFGATGG |
| trifasciata | | ASAGLISRVANALANTSTLRTVLRTGVSQQIASSVVQRAAQSLASTL |
| | | GVDGNNLARFAVQAVSRLPAGSDTSAYAQAFSSALFNAGVLNASNID |
| | | TLGSRVLSALLNGVSSAAQGLGINVDSGSVQSDISSSSSFLSTSSSS |
| | | ASYSQASASSTS |
| | | (SEQ ID NO: 36) |
| Uloborus diversus | AcSp | GASAADIATAIAASVATSLQSNGVLTASNVSQLSNQLASYVSSGLSS |
| | | TASSLGIQLGASLGAGFGASAGLSASTDISSSVEATSASTLSSSASS |
| | | TSVVSSINAQLVPALAQTAVLNAAFSNINTQNAIRIAELLTQQVGRQ |
| | | YGLSGSDVATASSQIRSALYSVQQGSASSAYVSAIVGPLITALSSRG |
| | | VVNASNSSQIASSLATAILQFTANVAPQFGISIPTSAVQSDLSTISQ |
| | | SLTAISSQTSSSVDSSTSAFGGISGPSGPSPYGPQPSGPTFGPGPSL |
| | | SGLTGFTATFASSFKSTLASSTQFQLIAQSNLDVQTRSSLISKVLIN |
| | | ALSSLGISASVASSIAASSSQSLLSVSA |
| | | (SEQ ID NO: 37) |
| Euprosthenops | MaSp1 | GGQGGQGQGRYGQGAGSSAAAAAAAAAAAAAA |
| australis | | (SEQ ID NO: 38) |
| Tetragnatha | MaSp1 | GGLGGGQGAGQGGQQGAGQGGYGSGLGGAGQGASAAAAAAAA |
| kauaiensis | | (SEQ ID NO: 39) |
| Argiope aurantia | MaSp2 | GGYGPGAGQQGPGSQGPGSGGQQGPGGLGPYGPSAAAAAAAA |
| | | (SEQ ID NO: 40) |
| Deinopis spinosa | MaSp2 | GPGGYGGPGQQGPGQGQYGPGTGQQGQGPSGQQGPAGAAAAAAAAA |
| | | (SEQ ID NO: 41) |
| Nephila clavata | MaSp2 | GPGGYGLGQQGPGQQGPGQQGPAGYGPSGLSGPGGAAAAAAA |
| | | (SEQ ID NO: 42) |

Fiber-forming block copolymer polypeptides from the blocks and/or macro-repeat domains, according to certain embodiments of the invention, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers include natural amino acid sequence information and other modifications described herein. The repeat domain is decomposed into repeat sequences containing representative blocks, usually 1-8 depending upon the type of silk, that capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a Glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macro-repeats.

In some embodiments, non-repetitive N- and C-terminal domains can be selected for synthesis. In some embodiments, N-terminal domains can be by removal of the leading signal sequence, e.g., as identified by SignalP (Peterson, T. N., et al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3× FLAG nucleotide coding sequence. In some embodiments, the silk polypeptide nucleotide coding sequence is operatively linked to other affinity tags such as 6-8 His residues. An example of a vector for delivering a silk polypeptide nucleotide coding sequence to a host cell is shown in FIG. 5, which depicts a plasmid map of a vector comprising an open reading frame (ORF) for expressing a silk-like polypeptide comprising a secretion signal and a FLAG tag. The ORF is operably linked to a pGCW14 promoter and a tAOX1 terminator. The vector also comprises selection markers to select successfully transformed cells.

In some embodiments, the silk polypeptide comprises a full length spider silk polypeptide. In some embodiments, the full length spider silk polypeptide is Major ampullate spidron 1 (MaSp1) or Major ampullate spidroin 2 (MaSp2).

### Silk-Like Polypeptides

In some embodiments, the P. pastoris strains disclosed herein have been modified to express a silk-like polypeptide. Methods of manufacturing preferred embodiments of silk-like polypeptides are provided in WO 2015/042164, especially at Paragraphs 114-134, incorporated herein by reference. Disclosed therein are synthetic proteinaceous copolymers based on recombinant spider silk protein fragment sequences derived from MaSp2, such as from the species Argiope bruennichi. Silk-like polypeptides are described that include two to twenty repeat units, in which a molecular weight of each repeat unit is greater than about 20 kDa. Within each repeat unit of the copolymer are more than about 60 amino acid residues that are organized into a number of “quasi-repeat units.” In some embodiments, the repeat unit of a polypeptide described in this disclosure has at least 95% sequence identity to a MaSp2 dragline silk protein sequence.

In some embodiments, each “repeat unit” of a silk-like polypeptide comprises from two to twenty “quasi-repeat” units (i.e., n3 is from 2 to 20). Quasi-repeats do not have to be exact repeats. Each repeat can be made up of concatenated quasi-repeats. Equation 1 shows the composition of a repeat unit according the present disclosure and that incorporated by reference from WO 2015/042164. Each silk-like polypeptide can have one or more repeat units as defined by Equation 1.

(Equation 1)
(SEQ ID NO: 13)
{GGY-[GPG-X₁]ₙ₁-GPS-(A)ₙ₂}ₙ₃.

The variable compositional element X₁ (termed a “motif”) is according to any one of the following amino acid sequences shown in Equation 2 and X₁ varies randomly within each quasi-repeat unit.

(Equation 2)
(SEQ ID NO: 14)
X₁ = SGGQQ
or
(SEQ ID NO: 15)
GAGQQ
or
(SEQ ID NO: 16)
GQGPY
or
(SEQ ID NO: 17)
AGQQ
or
SQ

Referring again to Equation 1, the compositional element of a quasi-repeat unit represented by “GGY-[GPG-X₁]n1-GPS” (SEQ ID NO: 18) in Equation 1 is referred to a “first region.” A quasi-repeat unit is formed, in part by repeating from 4 to 8 times the first region within the quasi-repeat unit. That is, the value of n1 indicates the number of first region units that are repeated within a single quasi-repeat unit, the value of n1 being any one of 4, 5, 6, 7 or 8. The compositional element represented by “(A) n2” (SEQ ID NO: 19) (i.e., a polyA sequence) is referred to as a “second region” and is formed by repeating within each quasi-repeat unit the amino acid sequence “A” n2 times (SEQ ID NO: 19). That is, the value of n2 indicates the number of second region units that are repeated within a single quasi-repeat unit, the value of n2 being any one of 6, 7, 8, 9, 10, 11, 12, 13, 14, 15, 16, 17, 18, 19, or 20. In some embodiments, the repeat unit of a polypeptide of this disclosure has at least 95% sequence identity to a sequence containing quasi-repeats described by Equations 1 and 2. In some embodiments, the repeat unit of a polypeptide of this disclosure has at least 80%, or at least 90%, or at least 95%, or at least 99% sequence identity to a sequence containing quasi-repeats described by Equations 1 and 2.

In additional embodiments, 3 “long” quasi repeats are followed by 3 “short” quasi-repeat units. Short quasi-repeat units are those in which ni=4 or 5. Long quasi-repeat units are defined as those in which n1=6, 7 or 8. In some embodiments, all of the short quasi-repeats have the same X₁ motifs in the same positions within each quasi-repeat unit of a repeat unit. In some embodiments, no more than 3 quasi-repeat units out of 6 share the same X₁ motifs.

In additional embodiments, a repeat unit is composed of quasi-repeat units that do not use the same X₁ more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X₁ more than 2 times in a single quasi-repeat unit of the repeat unit.

Thus, in some embodiments, provided herein are strains of yeast that recombinantly express silk-like polypeptides with a reduced degradation to increase the amount of full-length polypeptides present in the isolated product from a cell culture. In some embodiments, the strain expressing a silk-like polypeptide is a P. pastoris strain comprises a PAS_chr4_0584 knock-out and a PAS_chr3_1157 knock-out. In some embodiments, the strain expressing a silk-like polypeptide is a P. pastoris strain comprising an sec72 knock-out and/or an overexpressed SSH1 translocon complex.

TABLE 5
18B vector

| | SEQ | |
|---|---|---|
| | ID | |
| Description | NO: | 5′ to 3′ Sequence |
| 18B silk-like | 20 | ggtggttacg gtccaggcgc tggtcaacaa ggtccaggaa gtggtggtca acaaggacct 60 |
| polypeptide | | ggcggtcaag gaccctacgg tagtggccaa caaggtccag gtggagcagg acagcagggt 120 |
| encoding | | ccgggaggcc aaggacctta cggaccaggt gctgctgctg ccgccgctgc cgctgccgga 180 |
| sequence | | ggttacggtc caggagccgg acaacagggt ccaggtggag ctggacaaca aggtccagga 240 |
| | | tcacaaggtc ctggtggaca aggtccatac ggtcctggtg ctggtcaaca gggaccaggt 300 |
| | | agtcaaggac ctggttcagg tggtcagcag ggtccaggag gacagggtcc ttacggccct 360 |
| | | tctgccgctg cagcagcagc cgctgccgca ggaggatacg gacctggtgc tggacaacga 420 |
| | | tctcaaggac caggaggaca aggtccttat ggacctggcg ctggccaaca aggacctggt 480 |
| | | tctcagggtc caggttcagg aggccaacaa ggcccaggag gtcaaggacc atacggacca 540 |
| | | tccgctgcgg cagctgcagc tgctgcaggt ggatatggcc caggagccgg acaacagggt 600 |
| | | cctggttcac aaggtccagg atctggtggt caacagggac caggcggcca gggaccttat 660 |
| | | ggtccaggag ccgctgcagc agcagcagct gttggaggtt acggccctgg tgccggtcaa 720 |
| | | caaggcccag gatctcaggg tcctggatct ggaggacaac aaggtcctgg aggtcagggt 780 |
| | | ccatacggac cttcagcagc agctgctgct gcagccgctg gtggttatgg acctggtgct 840 |
| | | ggtcaacaag gaccgggttc tcagggtccg ggttcaggag gtcagcaggg ccctggtgga 900 |
| | | caaggacctt atggacctag tgcggctgca gcagctgccg ccgcaggtgg ttacggtcca 960 |
| | | ggcgctggtc aacaaggtcc aggaagtggt ggtcaacaag gacctggcgg tcaaggaccc 1020 |
| | | tacggtagtg gccaacaagg tccaggtgga gcaggacagc agggtccggg aggccaagga 1080 |
| | | ccttacggac caggtgctgc tgctgccgcc gctgccgctg ccggaggtta cggtccagga 1140 |
| | | gccggacaac agggtccagg tggagctgga caacaaggtc caggatcaca aggtcctggt 1200 |
| | | ggacaaggtc catacggtcc tggtgctggt caacagggac caggtagtca aggacctggt 1260 |
| | | tcaggtggtc agcagggtcc aggaggacag ggtccttacg gcccttctgc cgctgcagca 1320 |
| | | gcagccgctg ccgcaggagg atacggacct ggtgctggac aacgatctca aggaccagga 1380 |
| | | ggacaaggtc cttatggacc tggcgctggc caacaaggac ctggttctca gggtccaggt 1440 |
| | | tcaggaggcc aacaaggccc aggaggtcaa ggaccatacg gaccatccgc tgcggcagct 1500 |
| | | gcagctgctg caggtggata tggcccagga gccggacaac agggtcctgg ttcacaaggt 1560 |
| | | ccaggatctg gtggtcaaca gggaccaggc ggccagggac cttatggtcc aggagccgct 1620 |
| | | gcagcagcag cagctgttgg aggttacggc cctggtgccg gtcaacaagg cccaggatct 1680 |
| | | cagggtcctg gatctggagg acaacaaggt cctggaggtc agggtccata cggaccttca 1740 |
| | | gcagcagctg ctgctgcagc cgctggtggt tatggacctg gtgctggtca acaaggaccg 1800 |
| | | ggttctcagg gtccgggttc aggaggtcag cagggccctg gtggacaagg accttatgga 1860 |
| | | cctagtgcgg ctgcagcagc tgccgccgca ggtggttacg gtccaggcgc tggtcaacaa 1920 |
| | | ggtccaggaa gtggtggtca acaaggacct ggcggtcaag gaccctacgg tagtggccaa 1980 |
| | | caaggtccag gtggagcagg acagcagggt ccgggaggcc aaggacctta cggaccaggt 2040 |
| | | gctgctgctg ccgccgctgc cgctgccgga ggttacggtc caggagccgg acaacagggt 2100 |
| | | ccaggtggag ctggacaaca aggtccagga tcacaaggtc ctggtggaca aggtccatac 2160 |
| | | ggtcctggtg ctggtcaaca gggaccaggt agtcaaggac ctggttcagg tggtcagcag 2220 |
| | | ggtccaggag gacagggtcc ttacggccct tctgccgctg cagcagcagc cgctgccgca 2280 |
| | | ggaggatacg gacctggtgc tggacaacga tctcaaggac caggaggaca aggtccttat 2340 |
| | | ggacctggcg ctggccaaca aggacctggt tctcagggtc caggttcagg aggccaacaa 2400 |
| | | ggcccaggag gtcaaggacc atacggacca tccgctgcgg cagctgcagc tgctgcaggt 2460 |
| | | ggatatggcc caggagccgg acaacagggt cctggttcac aaggtccagg atctggtggt 2520 |
| | | caacagggac caggcggcca gggaccttat ggtccaggag ccgctgcagc agcagcagct 2580 |
| | | gttggaggtt acggccctgg tgccggtcaa caaggcccag gatctcaggg tcctggatct 2640 |
| | | ggaggacaac aaggtcctgg aggtcagggt ccatacggac cttcagcagc agctgctgct 2700 |
| | | gcagccgctg gtggttatgg acctggtgct ggtcaacaag gaccgggttc tcagggtccg 2760 |
| | | ggttcaggag gtcagcaggg ccctggtgga caaggacctt atggacctag tgcggctgca 2820 |
| | | gcagctgccg ccgca 2835 |
| 18B | 21 | GGYGPGAGQQGPGSGGQQGPGGQGPYGSGQQGPGGAGQQGPGGQGPYGPGAAAAAAAAAGGYGPGAGQQG |
| polypeptide | | PGGAGQQGPGSQGPGGQGPYGPGAGQQGPGSQGPGSGGQQGPGGQGPYGPSAAAAAAAAAGGYGPGAGQR |
| sequence | | SQGPGGQGPYGPGAGQQGPGSQGPGSGGQQGPGGQGPYGPSAAAAAAAAGGYGPGAGQQGPGSQGPGSGG |
| | | QQGPGGQGPYGPGAAAAAAAVGGYGPGAGQQGPGSQGPGSGGQQGPGGQGPYGPSAAAAAAAAGGYGPGA |
| | | GQQGPGSQGPGSGGQQGPGGQGPYGPSAAAAAAAAGGYGPGAGQQGPGSGGQQGPGGQGPYGSGQQGPGG |
| | | AGQQGPGGQGPYGPGAAAAAAAAAGGYGPGAGQQGPGGAGQQGPGSQGPGGQGPYGPGAGQQGPGSQGPG |
| | | SGGQQGPGGQGPYGPSAAAAAAAAAGGYGPGAGQRSQGPGGQGPYGPGAGQQGPGSQGPGSGGQQGPGGQ |
| | | GPYGPSAAAAAAAAGGYGPGAGQQGPGSQGPGSGGQQGPGGQGPYGPGAAAAAAAVGGYGPGAGQQGPGS |
| | | QGPGSGGQQGPGGQGPYGPSAAAAAAAAGGYGPGAGQQGPGSQGPGSGGQQGPGGQGPYGPSAAAAAAAA |
| | | GGYGPGAGQQGPGSGGQQGPGGQGPYGSGQQGPGGAGQQGPGGQGPYGPGAAAAAAAAAGGYGPGAGQQG |
| | | PGGAGQQGPGSQGPGGQGPYGPGAGQQGPGSQGPGSGGQQGPGGQGPYGPSAAAAAAAAAGGYGPGAGQR |
| | | SQGPGGQGPYGPGAGQQGPGSQGPGSGGQQGPGGQGPYGPSAAAAAAAAGGYGPGAGQQGPGSQGPGSGG |
| | | QQGPGGQGPYGPGAAAAAAAVGGYGPGAGQQGPGSQGPGSGGQQGPGGQGPYGPSAAAAAAAAGGYGPGA |
| | | GQQGPGSQGPGSGGQQGPGGQGPYGPSAAAAAAAA |
| Repeat | 22 | GGYGPGAGQQGPGSGGQQGPGGQGPYGSGQQGPGGAGQQGPGGQGPYGPGAAAAAAAAAGGYGPGAGQQG |
| sequence of a | | PGGAGQQGPGSQGPGGQGPYGPGAGQQGPGSQGPGSGGQQGPGGQGPYGPSAAAAAAAAAGGYGPGAGQR |
| silk-like | | SQGPGGQGPYGPGAGQQGPGSQGPGSGGQQGPGGQGPYGPSAAAAAAAAGGYGPGAGQQGPGSQGPGSGG |
| polypeptide | | QQGPGGQGPYGPGAAAAAAAVGGYGPGAGQQGPGSQGPGSGGQQGPGGQGPYGPSAAAAAAAAGGYGPGA |
| | | GQQGPGSQGPGSGGQQGPGGQGPYGPSAAAAAAAA |

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992).

### Example 1: Production of Recombinant Yeast Expressing 18B

Strains of P. pastoris were modified to recombinantly express 18B silk-like polypeptides as follows:

First, we transformed a strain of P. pastoris to abrogate KU70 function to facilitate further editing and engineering. A HIS+ derivative of Pichia pastoris (Komagataella phaffii) strain GS115 (NRRL Y15851) was electroporated with a DNA cassette consisting of homology arms flanking a zeocin resistance marker and targeting the KU70 locus. Sequences are provided in Table 12. Transformants were plated on YPD agar plates supplemented with zeocin. This resulted in abrogation of KU70 function.

Then, we modified this strain to express a recombinant gene encoding a silk-like polypeptide. A HIS+ derivative of Pichia pastoris (Komagataella phaffii) strain GS115 (NRRL Y15851) was transformed with a recombinant vector (SEQ ID NO: 20) to cause expression and secretion of a silk-like polypeptide (“18B”) (SEQ ID NO: 21). Transformation was accomplished by electroporation as described in PMID 15679083, incorporated by reference herein.

Each vector includes an 18B expression cassette with the polynucleotide sequence encoding the silk-like protein in the recombinant vectors flanked by a promoter (pGCW14) and a terminator (tAOX1 pA signal). The recombinant vectors further comprised dominant resistance markers for selection of bacterial and yeast transformants, and a bacterial origin of replication. The first recombinant vector included targeting regions that directed integration of the 18B polynucleotide sequences immediately 3′ of the AOX2 loci in the Pichia pastoris genome. The resistance marker in the first vector conferred resistance to G418 (aka geneticin). The second recombinant vector included targeting regions that directed integration of the 18B polynucleotide sequences immediately 3′ of the TEF1 loci in the Pichia pastoris genome. The resistance marker in the second vector conferred resistance to Hygromycin B.

### Example 2: Production of Recombinant ΔSec72 Strain

Cells modified to express 18B were transformed with a vector comprising a DNA cassette with 5′ and 3′ homology arms targeting sec72 (SEQ ID NO: 1). The homology arms flank a yeast selection marker, as shown in the plasmid map in FIG. 2, which knocks out the sec72 gene when inserted into the sec72 gene. Transformants were plated on YPD agar plates supplemented with yeast selection medium, and incubated for 48 hours at 30° C.

### Example 3: Production of Recombinant ΔSec72Strain Overexpressing an SSH1 Translocon Complex

P. pastoris (Δsec72) cells modified to express 18B (SEQ ID NO: 21) were transformed with a vector for overexpression of the SSH1 translocon complex. A plasmid map of the vector is shown in FIG. 3. The vector comprises open reading frames for SSH1 (SEQ ID NO: 3), SSS1 (SEQ ID NO: 5), and SBH2 (SEQ ID NO: 7). Each open reading frame is operatively linked to a promoter and a terminator. Transformants were plated on YPD agar plates supplemented with yeast selection medium, and incubated at 48 hours at 30° C.

### Example 4: Production of Protease Double Knock-Out Strain

To generate AAprotease strains (i.e., double protease knock-out), a selected yeast strain was transformed with vector comprising a DNA cassette with ˜1150 bp homology arms flanking a nourseothricin resistance marker. A plasmid map comprising the nourseothricin resistance marker is shown in FIG. 4A, and sequences provided in Table 13. Homology arms used for each target were amplified by the primers provided in Table 8, and inserted into the nourseothricin resistance plasmid. Homology arms were inserted into the nourseothricin plasmid to generate cassettes comprising a nourseothricin resistance marker flanded by 3′ and 5′ homology arms to the target protease as shown in FIG. 4B and FIG. 4C. In FIG. 4B, the resistance cassette (Nour Resistance Cassette) is shown flanked by homology arms (HA1 and HA2). In FIG. 4C, details of the nourseothricin marker are shown, including the promoter from ILV5 gene from Saccharomyces cerevisiae (pILV5), the Nourseothricin acetyltransferase gene from Streptomyces noursei (nat), and the polyA signal from CYC1 gene from Saccharomyces cerevisiae.

A vector with homology arms targeting YPS1-1 (SEQ ID NO: 77) was used to transform modified yeast strains. Transformants were plated on YPD agar plates supplemented with nourseothricin, and incubated for 48 hours at 30° C.

TABLE 6
Proteases targeted for deletion in P. pastoris strain.

| | Protease | Protease |
|---|---|---|
| | ORF | polypeptide |
| | Sequence | sequence |
| Protease Gene Symbol | (SEQ ID NO:) | (SEQ ID NO:) |
| PAS_chr4_0584 | 9 | 10 |
| (YPS1-1) | | |
| PAS_chr3_1157 | 11 | 12 |
| (YPS1-2) | | |

To generate double knockouts, nourseothricin resistance was eliminated from the single protease knock-out strains produced above. A vector with homology arms targeting YPS1-2 (SEQ ID NO: 80) was used to transform the single protease knock-out strains. Transformants were plated on YPD agar plates supplemented with nourseothricin, and incubated for 48 hours at 30° C.

### Example 5: ΔSec72 Improves MFa-18B Secretion Across Integration Copy Numbers

Pichia pastoris strains modified to express two, four, or six copies of 18B (Ab MaSp2 79 kDa) comprising the MFa1 (sc) pre-pro leader sequence were prepared using techniques described in Example 1.

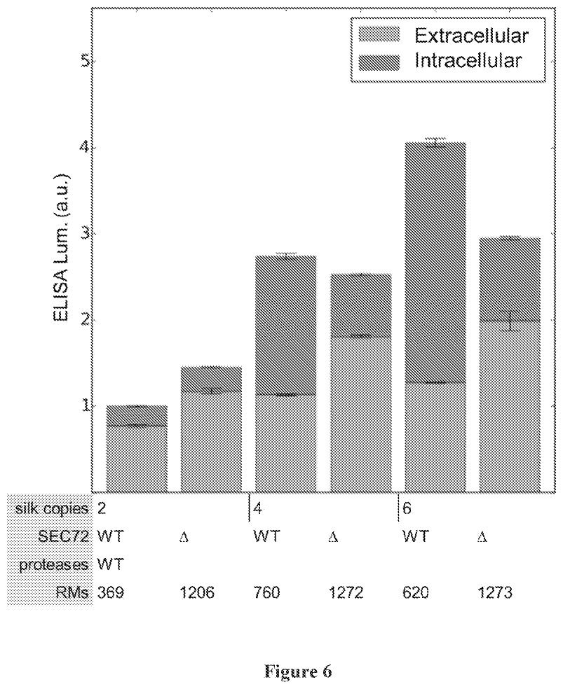

Δsec72 strains of each of the above were prepared using techniques described in Example 2. Δsec72 and WT strains for each of the 2×, 4×, and 6× 18B expressing strains were prepared. Secretion of MFa-18B from WT and Δsec72 strains (i.e, . . . SEC72 KO strains) from each of the 2×, 4× and 6× 18B expressing strains was measured by ELISA, with the results shown in FIG. 6.

Referring to FIG. 6, “silk copies” are the number of expression cassettes for 18B (Ab MaSp2 79 kDa) using the MFa1 (sc) pre-pro leader sequence in each strain. The 18B is C-terminally tagged with a 3×FLAG epitope for ELISA detection. Error bars show standard error of the mean among n≥4 biological replicates. The results show that deletion of the sec72 gene improves MFa-18B secretion across integration copy numbers (i.e., 2×, 4×, and 6× silk copies).

### Example 6: ΔSec72Improves Secretion from Non-MFa Signals

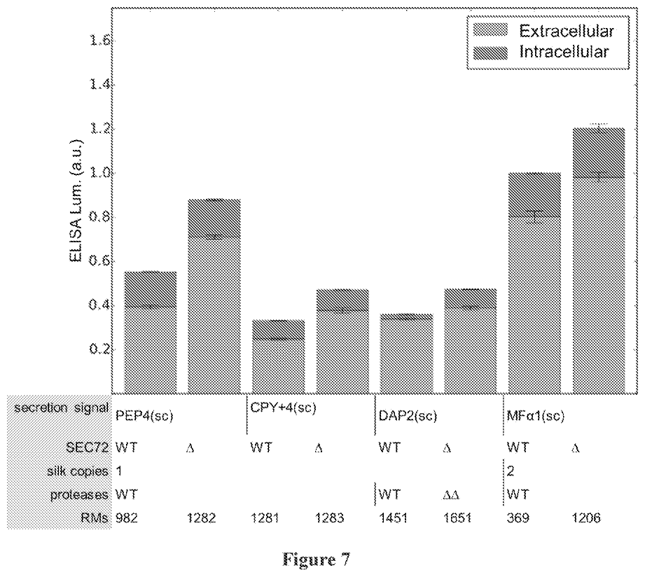

P. pastoris strains were modified according to Example 1 to express 4 different silk polypeptides each comprising a different secretion signal: PEP4 (sc), CPY+4 (sc), DAP2 (sc), and MFa1 (sc). Each complete leader sequence is a hybrid composed of the indicated signal peptide with the MFa1 (sc) propeptide (S. cerevisiae ortholog systematic name: YPL187W). (sc) indicates the signal peptide derives from S. cerevisiae. Strains recombinantly expressing polypeptides with PEP4 (sc), CPY+4 (sc), and DAP2 (sc) each comprise a single expression cassette (i.e., 1 silk copy). Strains recombinantly expressing MFa1 (sc) comprise 2 expression cassettes (i.e., 2 silk copies). Δsec72 strains of each of the above were prepared using techniques described in Example 2 to compare secretion from WT and Δsec72 strains. For the Δsec72strain comprising DAP2 (sc), the strain was additionally modified to knockout YPS1-1 and YPS1-2 proteases, as described in Example 4. Secretion of polypeptides with each secretion signal from WT and Δsec72 strains was measured by ELISA, with the results shown in FIG. 7.

The results indicate that the deletion of the sec72 gene improves secretion for polypeptides comprising non-MFa1 (sc) signal peptides. Error bars show standard error of the mean among n≥4 biological replicates.

TABLE 7A
MFα1(sc) propeptide sequence

| | | SEQ |
|---|---|---|
| | | ID |
| | Sequence | NO: |
| MFα1(sc) | gctccagtcaacactacaacagaagatga | 105 |
| propeptide | aacggcacaaattccggctgaagctgtca | |
| Nucleotide | tcggttactcagatttagaaggggatttc | |
| Encoding | gatgttgctgttttgccattttccaacag | |
| Sequence | cacaaataacgggttattgtttataaata | |
| (5′ to 3′) | ctactattgccagcattgctgctaaagaa | |
| | gaaggggtatctctcgagaaaagagaggc | |
| | tgaa | |
| MFα1(sc) | APVNTTTEDETAQIPAEAVIGYSDLEGDF | 106 |
| propeptide | DVAVLPFSNSTNNGLLFINTTIASIAAKE | |
| Polypeptide | EGVSLEKREAE | |
| Sequence | | |

TABLE 7B
Silk Secretion Signals

| | | SEQ |
|---|---|---|
| Secretion | | ID |
| Signal | Sequence | NO: |
| PEP4 | atgttcagcttgaaagcattattgccatt | 83 |
| Nucleotide | ggccttgttgttggtcagcgccaaccaag | |
| Encoding | ttgctgca | |
| Sequence | | |
| (5′ to 3′) | | |
| PEP4 | MFSLKALLPLALLLVSANQVAA | 84 |
| Polypeptide | | |
| Sequence | | |
| PRC1, mutant | atgaaagcattcctgttgttactactttt | 85 |
| (i.e., | actaggcctgtccactacactcgctaagg | |
| CPY + 4) | ca | |
| Nucleotide | | |
| Encoding | | |
| Sequence | | |
| (5′ to 3′) | | |
| PRC1, mutant | MKAFLLLLLLLGLSTTLAKA | 86 |
| (i.e., | | |
| CPY + 4) | | |
| Polypeptide | | |
| Sequence | | |
| DAP2 | atggaaggtggcgaagaagaagttgagcg | 87 |
| Nucleotide | cattcctgatgaacttttcgatacaaaaa | |
| Encoding | agaagcatttgttagataagctcataagg | |
| Sequence | gtcggaataatccttgtactcctgatatg | |
| (5′ to 3′) | gggcactgttttgttgctaaaaagtatt | |
| DAP2 | MEGGEEEVERIPDELFDTKKKHLLDKLIR | 88 |
| Polypeptide | VGIILVLLIWGTVLLLKSI | |
| Sequence | | |
| MF(alpha) 1, | atgagatttccttcaatttttactgctgt | 89 |
| variant | tttattcgcagcatcctccgcattagct | |
| Nucleotide | | |
| Encoding | | |
| Sequence | | |
| (5′ to 3′) | | |
| MF(alpha) 1, | MRFPSIFTAVLFAASSALA | 90 |
| variant | | |
| Polypeptide | | |
| Sequence | | |

### Example 7: ΔSec72 Improves Secretion of Longer Silks and Distinct Silk Sequences

P. pastoris strains were modified according to Example 1 to express long silk polypeptides Argioppe bruennichi (Ab) MaSp2 (106 kDa) (SEQ ID NO: 43),Latrodectus hesperus (Lh) MaSp1 (55 kDa) (SEQ ID NO: 44). Ab MaSp2 106 kDa (aka 24B) is a longer concatemer of Ab MaSp2 79 kDa (18B). Lh MaSp1 55 kDa is distinct sequence from another class of spidroins.

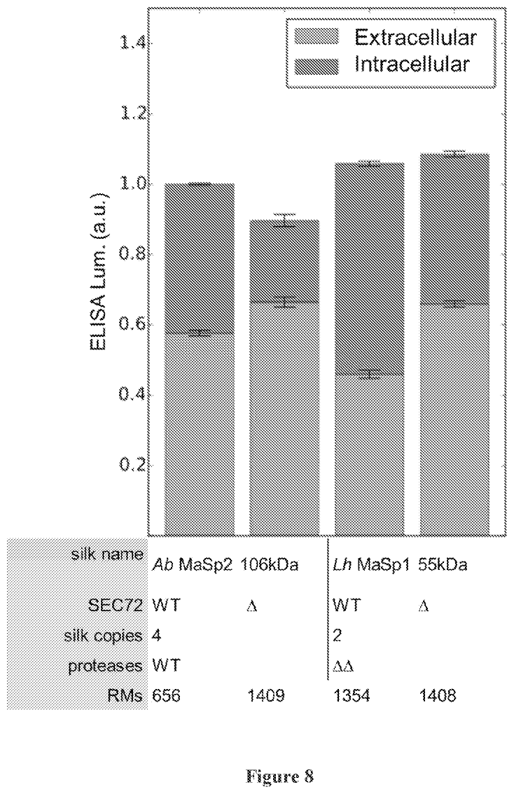

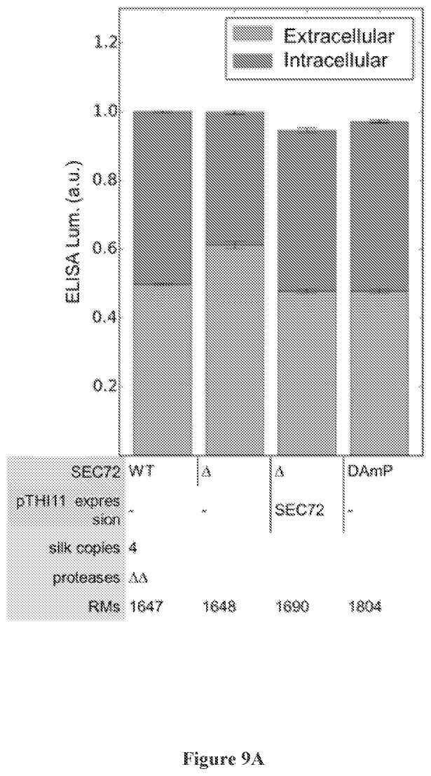

Δsec72 strains of each were prepared using techniques described in Example 2 to compare secretion from WT and Δsec72 strains. The strains comprising Lh MaSp1 (55 kDa) were additionally modified to knockout YPS1-1 and YPS1-2 proteases, as described in Example 4. Secretion of the long silk polypeptides from WT and Δsec72 strains was measured by ELISA, with the results shown in FIG. 8. Error bars show standard error of the mean among n≥4 biological replicates.

Argioppe bruennichi MaSp2 protein amino acid
sequence (SEQ ID NO: 43):
MNWSIRLALLGFVVLSTQTVFAVGQAATPWENSQLAEDFINSFLRFIAQS
GAFSPNQLDDMSSIGDTLKTAIEKMAQSRKSSKSKLQALNMAFASSMAEI
AVAEQGGLSLEAKTNAIANALASAFLETTGFVNQQFVSEIKSLIYMIAQA
SSNEISGSAAAAGGGSGGGGGSGQGGYGQGASASASAAAAYGSAPQGAGG
PAPQGPSQQGPVSQGPYGPGAAAAAAAAGGYGPGAGQQGPGSQGPGSGGQ
QGPGSQGPGSGGQQGPGGQGPYGPSAAAAAAAAAGGYGPGAGQQGPGSQG
PGSGGQQGPGGQGPYGPGAAAAAAAVGGYGPGAGQQGPGSQGPGSGGQQG
PGGQGPYGPSAAAAAAAAGGYGPGAGQQGPGSQGPGSGGQQGPGGQGPYG
PSAAAAAAAAGGYGPGAGQQGPGSGGQQGPGGQGPYGSGQQGPGGAGQQG
PGGQGPYGPGAAAAAAAAAGGYGPGAGQQGPGGAGQQGPGSQGPGGQGPY
GPGAGQQGPGSQGPGSGGQQGPGGQGPYGPSAAAAAAAAGGYGPGAGQQG
PGSQGPGSGGQQGPGSQGPGSGGQQGPGGQGPYGPSAAAAAAAAGGYGPG
AGQQGPGSQGPGSGGQQGPGGQGPYGPGAAAAAAAVGGYGPGAGQQGPGS
QGPGSGGQQGPGGQGPYGPSAAAAAAAAGGYGPGAGQQGPGSQGPGSGGQ
QGPGGQGPYGPSAAAAAAAAGGYGPGAGQQGPGSGGQQGPGGQGPYGSGQ
QGPGGAGQQGPGGQGPYGPGAAAAAAAAAGGYGPGAGQQGPGGAGQQGPG
SQGPGGQGPYGPGAGQQGPGSQGPGSGGQQGPGGQGPYGPSAAAAAAAAA
GGYGPGAGQRSQGPGGQGPYGPGAGQQGPGSQGPGSGGQQGPGGQGPYGP
SAAAAAAAAGPGAGRQGPGSQGPGSGGQQGPGGQGPYGPSAAAAAAAAGP
GARRQGPGSQGPGSGGQQGPGGQGPYGSGQQGPGGAGQQGPGGQGPYGPG
AAAAAAAAAGGYGPGAGQQGPGGAGQQGPGSQGPGGQGPYGPGAGQQGPG
SQGPGSGGQQGPGGQGPYGPSAAAAAAAAGGYGPGAGQQGPGSQGPGSGG
QQGPGGQGPYGPGAAAAAAAVGGYGPGAGQQGPGSQGPGSGGQQGPGGQG
PYGPSAAAAAAAAGGYGPGAGQQGPGSQGPGSGGQQGPGGQGPYGPSAAA
AAAAAGGYGPGAGQQGPGSGGQQGPGGQGPYGSGQQGPGGAGQQGPGGQG
PYGPGAAAAAAAAAGGYGPGAGQQGPGGAGQQGPEGPGSQGPGSGGQQGP
GGQGPYGPGAAAAAAAVGGYGPGAGQQGPGSQGPGSGGQQGPGGQGPYGP
SAAAAAAAAGGYGPGAGQQGPGSQGPGSGGQQGPGGQGPYGPSAAAAAAA
AGGYGPGAGQQGPGSGGQQGPGGQGPYGSGQQGPGGAGQQGPGGQGPYGP
GAAAAAAAAAGGYGPGAGQQGPGGAGQQGPGSQGPGGQGPYGPGAGQQGP
GSQGPGSGGQQGPGGQGPYGPSAAAAAAAAGGYGPGAGQQGPGSQGPGSG
GQQGPGSQGPGSGGQQGPGGQGPYGPSAAAAAAAAAGGYGPGAGQQGPGS
QGPGSGGQQGPGGQGPYGPGAAAAAAAVGGYGPGAGQQGPGSQGPGSGGQ
QGPGGQGPYGPSAAAAAAAAGGYGPGAGQQGPGSQGPGSGGQQGPGGQGP
YGPSAAAAAAAAGGYGPGAGQQGPGSGGQQGPGGQGPYGSGQQGPGGAGQ
QGPGGQGPYGPGAAAAAAAAAGGYGPGAGQQGPGGAGQQGPGSQGPGGQG
PYGPGAGQQGPGSQGPGSGGQQGPGGQGPYGPSAAAAAAAAGGYGPGAGQ
QGPGSQGPGSGGQQGPGGQGPYGPSAAAAAAAAGGYGPGAGQQGPGSGGQ
QGPGGQGPYGSGQQGPGGAGQQGPGGQGPYGGGYGPGAGQQGPGSQGPGS
GGQQGPGGQGPYGPSAAAAAAAAGPGARRQGPGSQGPGSGGQQGPGGQGP
YGSGQQGPGGAGQQGPGGQGPYGPGAAAAAAAAAGGYGPGAGQQGPGGAG
QQGPGSQGPGGQGPYGPGAGQQGPGSQGPGSGGQQGPGGQGPYGPSAAAA
AAAAAGGYGPGAGQQGPGSQGPGSGGQQGPGGQGPYGPGAAAAAAAVGGY
GPGAGQQGPGSQGPGSGGQQGPGGQGPYGPSAAAAAAAAGGYGPGAGQQG
PGSQGPGSGGQQGPGGQGPYGPSAAAAAAAAGGYGPGAGQQGPGSGGQQG
PGGQGPYGSGQQGPGGAGQQGPGGQGPYGPGAAAAAAAAAGGYGPGAGQQ
GPGGAGQQGPGSQGPGGQGPYGPGAGQQGPGSQGPGSGGQQGPGGQGPYG
PSAAAAAAAAGPGAGRQGPGSQGPGSGGQQGPGGQGPYGPSAAAAAAAAG
PGARRQGPGSQGPGSGGQQGPGGQGPYGSGQQGPGGAGQQGPGGQGPYGP
GAAAAAAAAAGGYGPGAGQQGPGGAGQQGPGSQGPGGQGPYGPGAGQQGP
GSQGPGSGGQQGPGSQGPYGPSAAAAAAAAGGYGPGAGQQGPGSQGPGSG
GQQGPGGQGPYGPSAAAAAAAAGGYGPGAGQQGPGSGGQQGPGGQGPYGS
GQQGPGGAGQQGPGGQGPYGPGAAAAAAAAAGGYGPGAGQQGPGSQGPGS
GGQQGPGSQGPGSGGQQGPGGQGPYGPSAAAAAAAAAGGYGPGAGQQGPG
SQGPGSGGQQGPGGQGPYGPGAAAAAAAVGGYGPGAGQQGPGSQGPGSGG
QQGPGGQGPYGPSAAAAAAAAGGYGPGAGQQGPGSQGPGSGGQQGPGGQG
PYGPSAAAAAAAAGGYGPGAGQQGPGSGGQQGPGGQGPYGSGQQGPGGAG
QQGPGGQGPYGPGAAAAAAAAAGGYGPGAGQQGPGGAGQQGPGSQGPGGQ
GPYGPGAGQQGPGSQGPGSGGQQGPGGQGPYGPSAAAAAAAAGGYGPGAG
QQGPGSQGPGSGGQQGPGGQGPYGPGAAAAAAAVGGYGPGAGQQGPGSQG
PGSGGQQGPGGQGPYGPSAAAAAAAAGGYGPGAGQQGPGSQGPGSGGQQG
PGGQGPYGPSAAAAAAAAGGYGPGAGQQGPGSGGQQGPGGQGPYGSGQQG
PGGAGQQGPGGQGPYGPGAAAAAAAAAGGYGPGAGQQGPGGAGQQGPGSQ
GPGGQGPYGPGAGQQGPGSQGPGSGGQQGPGGQGPYGPSAAAAAAAAGGY
GPGAGQQGPGSQGPGSGGQQGPGGQGPYGPSAAAAAAAAGGYGPGAGQQG
PSSQAPVASAAASRLSSPQASARVSSAVSTLVSSGPTSPAALSNAISSVV
SQVSASNPGLSGCDVLVQALLEIVSALVHILGSSSIGQINYAASSQYAQM
VGNSVAQALG
Latrodectus hesperus MaSp1 protein amino acid
sequence (SEQ ID NO: 44):
MTWSTRLALSFLFVLCTQSLYALAQANTPWSSKANADAFINSFISAASNT
GSFSQDQMEDMSLIGNTLMAAMDNMGGRITPSKLQALDMAFASSVAEIAA
SEGGDLGVTTNAIADALTSAFYQTTGVVNSRFISEIRSLIGMFAQASAND
VYASAGSSGGGGYGASSASAASASAAAPSGVAYQAPAQAQISFTLRGQQP
VSYGQGGAGPGGAGAAAAAAAAAGGAGQGGQGGYGQGGYGQGGAGQGGSG
AAAAAAAAAGGTGQGGAGQGGAGAAAAAAAAAGGAGQGGQGGYGQGGYGQ
GGTGQGGAGAAAAAAAAGGAGQGGQGGYGQGGYGQGGYGQGGSGAAAAAA
AAAGGAGQGGQGGYGQGGYGQGGAGQGGAGAAAAAAAAAGGAGQGGYGRG
GAGQGGAAAAAAAAAGAGQGGYGGQGAGQGGSGAAAAAAAAGGAGQGGQG
GYGQGGYGQGGSGAAAAAAAAGGAGQGGQGGYGQGGYGQGGAGQGGAGAA
AAAAAAGGAGQGGQGGYGQGGYGQGGAGQGGAGAAAAAAAAGGAGQGGQG
GYGQGGYGQGGAGQGGAGAAAAAAAAGGAGQGGQGGYGQGGYGQGGAGQG
GAAAAAAAAAGGAGQGGYGRGGAGQGGAAAAAGAGQGGYGGQGAGQGGAG
AAAAAAAAGGAGQGGQGGYGRGGYGQGGAGQGGAGAAAAAAAAGGAGQGG
QGGYGQGGYGQGGAGQGGAAAAAAAAGGAGQGGYGRGGAGQGGAAAAAAA
AAGAGQGGYGGQGAGQGGAGAAAAAAAAGGAGQGGQGDYGRGGYGQGGAG
QGGAGAAAAAAAAGGAGQGGQGGYGQGGYGQGGAGQGGAAAAASAAAAGG
AGQGGYGRGGAGQGGAAAAAGAGQGGYGGQGAGQGGAGAAAAAAAAGGAG
QGGQGGYGRGGYGQGGAGQGGAGAAAAATAAGGAGQGGQGGYGQGGYGQG
GAGQGGAAAAAAAAAGGAGQGGYGRGGAGQGGAAAAAAAAAGAGQGGYGG
QGAGQGGAGAAAAAAGGAGQGGQGGYGRGGYGQGGAGQGGAGAAAAAAAA
GGAGQGGQGGYGQGGYGQGGAGQGGAAAAAAAAAGGAGQGGYGRGGAGQG
GAAAAAGAGQGGYGGQGAGQGGAGAAAAASRGAGQGGQGGYGRGGYGQGG
AGQGGAGAAAAAAAAGGAGQGGQGGYGQGGYGQGGAGQGGAAAAAAAAGG
AGQGGYGRGGAGQGGAAAAAGAGQGGYGGQGAGQGGAGAAAAAAAAGGAG
QGGQGGYGRGGYGQGGAGQGGAGAAAAAAAAGGAGQGGQGGYGQGGYGQG
GAGQGGAAAAAAAAAGGAGQGGYGRGGAGQGGAAAAAAAAAGSGQGGYGG
QGAGQGGAGAAAAAAAAGGAGQGGQGGYGRGGYGQGGAGQGGAGAAAAAA
AAGGAGQGGQGGYGQGGYGQGGAGQGGAAAAAAAAAAGGAGQGGYGRGGA
GQGGAAAAAGAGQGGYGGQGAGQGGAGAAAAAAAAGGAGQGGQGGYGRGG
YGQGGAGQGGAGTAAAAAAAGGAGQGGQGGYGQGGYGQGGAGQGGAAAAA
AAAAGGAGQGGYGRGGAGQGGAAAAAAAAAGAGQGGYGGQGAGQGGAGAA
AAAAAAGGAGQGGQGGYGRGGYGQGGAGQGGAGAAAAAAAAGGASQGGQG
GYGQGDYGQGGAGQGGAAAAAAAAGGAGQGGYGRGGAGQGGAAAAAGAGQ
GGYGGQGAGQGGAGAAAAAAAAGGAGRGGQGGYGRGGYGQGGAGQGGAGA
AAAAAAAGGAGQGGQGGYGQGGYGQGGTGQGGAAAAAAAAAGGAGQGGYG
RGGAGQGGAAAAAAAAAGAGQGGYGGQGAGQGGAGAAAAAAAAGGAGQGG
QGGYGRGGYGQGGAGQGGAGAAAAAAAAGGAGQGGQGGYGQGGYGQGGYG
QGGAGQGGAAAAAAAAGGAGQGGYGRGGAGQGGAAAAAGAGQGGYGGQGA
GQGGAGAAAAAAAAGGAGQGGQGGYGRGGYGQGGAGQGGAGAAAAAAAAG
GAGQGGQGGYGQGGYGQGGAGQGGAAAAAAAAAGGAGQGGYGRGGAGQGG
AAAAAAAAAGSGQGGYGGQGAGQGGAGAAAAAAAAGGAGQGGQGGYGRGG
YGQGGAGQGGAGAAAAAAAAGGAGQGGQGGYGQGGYGQGGYGQGGAGQGG
AAAAAAAAAAGGAGQGGYGRGGAGQGGAAAAAGAGQGGYGGQGAGQGGAG
AAAAAAAAGGAGQGGQGGYGRGGYGQGGAGQGGAGAAAAAAAAGGAGQGG
QGGYGQGGNGQGGAGQGGAAAAAAAAGGAGQGGYGRGGAGQGGAAAAAAA
AAGAGQGGYGGQGAGQGGAGAAAAAAAAGGAGQGGQGGYGRGGYGQGGAG
QGGAGAAAAAAAAGGASQGGQGGYGQGDYGQGGAGQGGAAAAAAAAGGAG
QGGYGRGGAGQGGAAAAAGAGQGGYGGQGAGQGGAGAAAAAAAAGGAGRG
GQGGYGRGGYGQGGAGQGGAGAAAAAAAAGGAGQGGQGGYGQGGYGQGGA
GQGGAAAAAAAAAGGAGQGGYGRGGAGQGGAAAAAGAGQGGYGGQGAGQG
GAGAAAAAAAAGGAGRGGQGGYGRGGYGQGGAGQGGAGAAAAAAAAGGAG
QGGQGGYGQGGYGQGGAGQGGAAAAAAAAVGGAGQGGYGRGGAGQGGAAA
AAAAAAAGSGQGGYGGQGAGQGGAGAAAAAAAAGGAGQGGQGGYGGGGYG
QGGAGQGGAGAAAAAAAAGGAGQGGQGGYGQGGYGQGGAGQGGAAAAAAA
AAGGAGQGGYGRGGAGQGGAAAATGAGQGGYGGQGAGQGGAGAAAAAAAA
GGAGQGGQGGYGRGGYGQGGAGQGGAGAAAAAAAAGGAGQGGQGGYGQGG
YGQGGAGQGGAAAAAAAAGGAGQGGYGRGGAGQGGAAAAAAAAAGAGQGG
YGGQGAGQGGAGAAAAAAAAGGAGQGGQGGYGRGGYGQGGAGQGGAGAAA
AGGAGQGGQGGYGQGGYGQGGAGQGGAAAAAAAAAAGGAGQGGYGGYGQQ
GGAGAAAAAASGPGQIYYGPQSVAAPAAAAASALAAPATSARISSHASAL
LSNGPTNPASISNVISNAVSQISSSNPGASACDVLVQALLELVTALLTII
GSSNIGSVNYDSSGQYAQVVTQSVQNAFA

### Example 8: Secretion Improves with SEC72 Deletion, but Neither Knockdown Nor Overexpression

P. pastoris strains were modified according to Example 1 to comprise 4 copies of DNA cassetttes comprising recombinant genes expressing 18B silk-like polypeptide. Δsec72 strains were prepared using techniques described in Example 2. The strains were additionally modified to knockout YPS1-1 and YPS1-2 proteases, as described in Example 4.

From this strain, a strain overexpressing sec72 was prepared by transforming with a vector comprising a recombinant sec72 gene operably linked to a THI11 promoter (pTHI11). pTHI11 is de-repressed in minimal media that lacks the vitamin thiamine. From prior RNAseq and promoter fusion studies, pTHI11 is among the strongest promoters in block and tank minimal media, which lack thiamine.

Additionally, from the P. pastoris strain modified to express 4 copies of recombinant genes expressing 18B silk-like polypeptide, instead of knocking out sec72, expression of SEC72 was knocked down using DAmP (decreased abundance by mRNA perturbation), a transcriptional knockdown strategy that disrupts the 3′ UTR of a gene with a marker cassette.

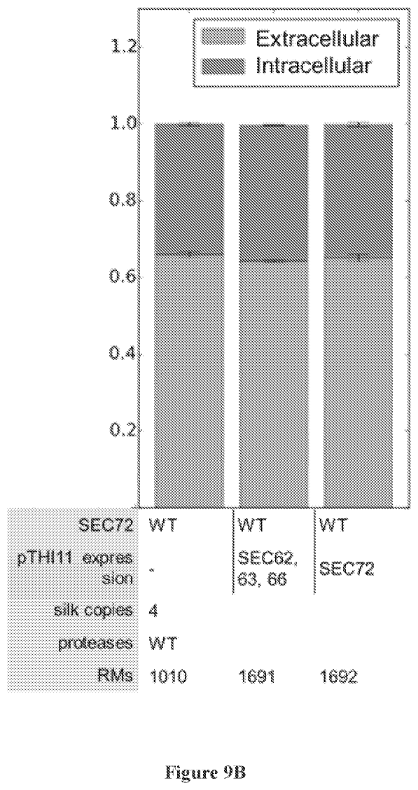

Secretion of the 18B silk-like polypeptides from WT, Δsec72, Δsec72 with recombinantly overexpressed sec72 (operably linked to pTHI11), and sec72 knockdown strains was measured by ELISA, with the results shown in FIG. 9A.

The Δsec72 phenotype reverts upon complementation with an inducible allele of sec72. Similarly, the knockdown of SEC72 did not measurably affect silk secretion as compared to WT. Thus, secretion improves with sec72 deletion, but not with knockdown nor overexpression of sec72.

### Example 9: Other Disruptions to SEC63 Complex Stoichiometry do not Affect Silk Secretion

P. pastoris strains were modified according to Example 1 to comprise 4 copies of DNA cassetttes comprising recombinant genes expressing 18B silk-like polypeptide. Δsec72 strains were prepared using techniques described in Example 2.

To test whether the Δsec72 phenotype is a direct effect of removal of SEC72 or an indirect effect of disrupting the SEC63 complex stoichiometry, pTHI11 was used to overexpress the 3 non-SEC72 complex members (SEC62, SEC63, and SEC66) simultaneously (by transforming the strain with a vector comprising 3 concatenated expression cassettes with sec62, sec63, and sec66). In addition, pTHI11 was used to overexpress SEC72. Secretion of 18B was measured from each strain and compared with Δsec72 strains using ELISA, with the results shown in FIG. 9B.

Neither stoichiometric change (overexpression of SEC62, SEC63, and SEC66, or overexpression of SEC72) measurably affected silk secretion. Error bars show standard error of the mean among n≥3 biological replicates.

### Example 10: RTqPCR Shows Transcriptional Adaptation to ΔSec72, Including Translocon Levels

Δsec72 strains were prepared using techniques described in Example 2. mRNA was isolated after growth and RTqPCR was performed on selected markers to analyze transcriptional adaptation to Δsec72.

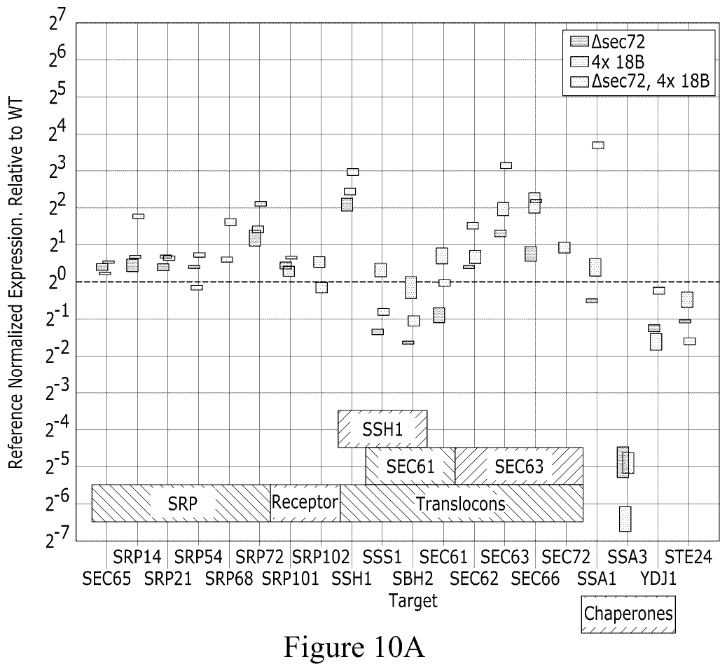

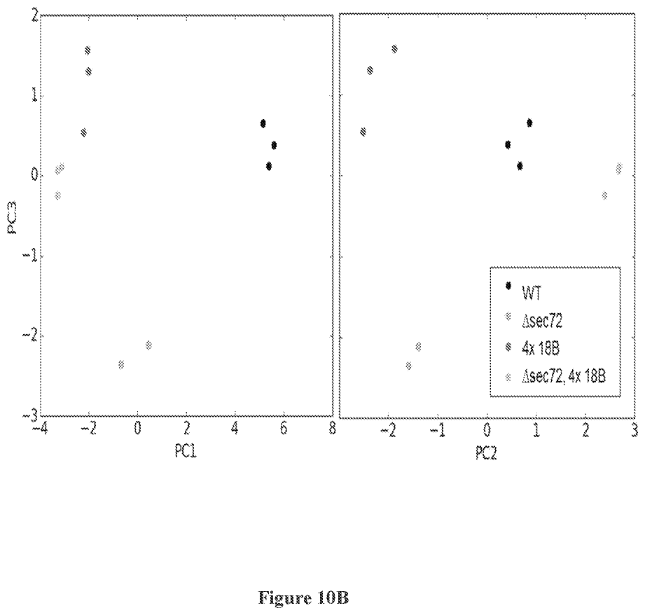

Reference normalized expression (ALG9 or ACT1) as a fold change from the “WT” strain RMs71 in minimal media is shown in FIG. 10A. Shading indicates range and center bar represents median among n=3 biological replicates (except n=2 for Δsec72). SSH1 transcript levels increase 4-8 fold across all 3 perturbations. Δsec72 leads to a 2-4 fold reduction in SEC61 translocon component expression.

FIG. 10B shows a projection of centered, log-scaled, reference normalized expression data onto principal component space, left=PC1 vs. 3, right=PC2 vs. 3. Replicates are colored by strain genotype, showing low within-genotype variance compared variance across genotypes. Examining the loading coefficients revealed that most variation along PC1 is driven by strong SSA3 repression and along PC3 by SEC61 translocon complex repression. These 3 PCs reconstruct 99% of the variance from the original 20 dimensions.

### Example 11: Overexpression of SSH1 Improves Growth Rate of the ΔSec72 Silk Secretion Strain

Though SSH1 also functions in ER translocation, it is distinct from SEC61 in its dispensability for S. cerevisiae growth and lack of interaction with the SEC63 complex. SSH1 may show translocation substrate preferences, but they broadly overlap with those of SEC61.

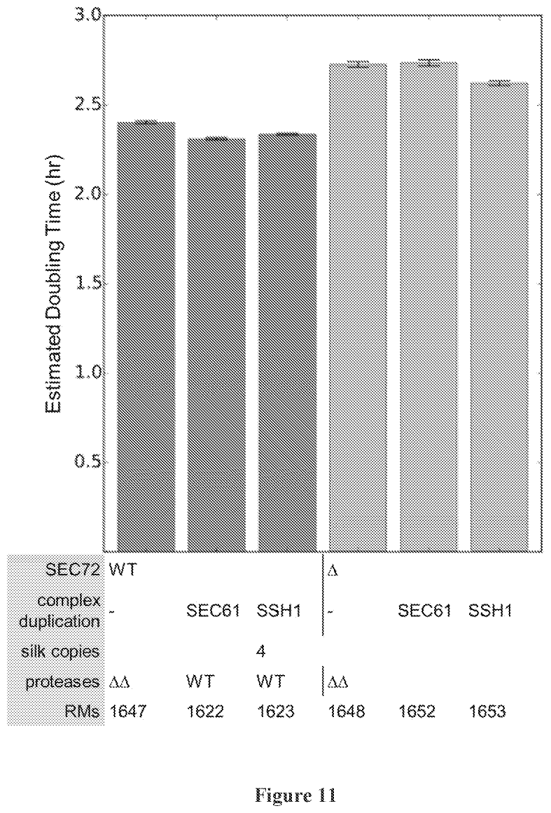

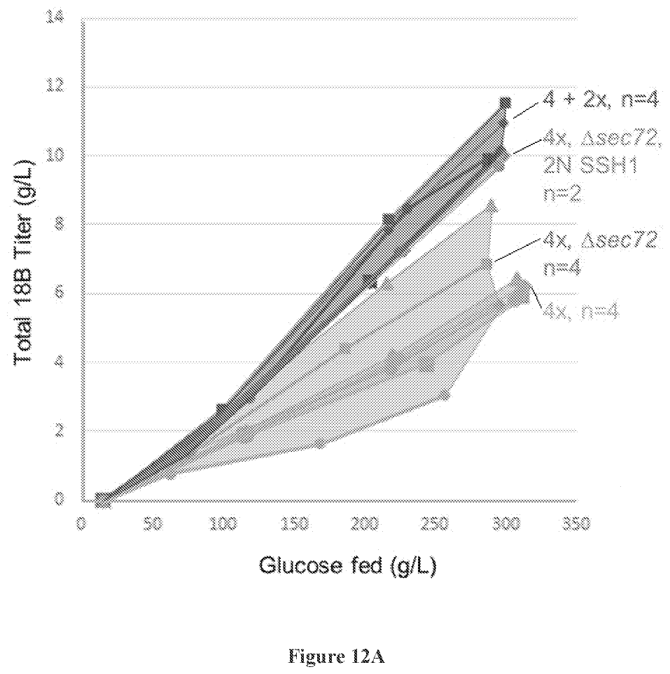

To test whether overexpression of SSH1 could aid growth of Δsec72 strains, P. pastoris Δsec72 strains were prepared using techniques described in Example 2. The regulatory and coding sequences for the SEC61 and SSH1 complexes (SSS1, SBH2, and one of SEC61 or SSH1) were assembled by PCR onto an integrating plasmid. The resulting integration duplicates the gene copy number of one of the SEC61 or SSH1 complexes. P. pastoris (Δsec72) cells were transformed with a vector for overexpression of the SSH1 translocon complex as described in Example 3. Similarly, P. pastoris (Δsec72) cells were transformed with a vector for overexpression of SEC61. Selected strains (as indicated by “AA” in FIG. 11) were additionally modified to knockout YPS1-1 and YPS1-2 proteases, as described in Example 4.

Pre-cultures in YPD were diluted 1:1600 into minimal media (RMm17). OD600 was recorded over 6 timepoints spanning 8 hours of exponential growth beginning 19 hours after dilution. A linear model fit log (OD600) vs. time to estimate doubling rate and the standard error of the slope among n=6 biological replicates. The measured growth for each strain is shown in FIG. 11. Doubling times are shown as the reciprocal of doubling rate. Error bars are asymmetrical due to the reciprocal transformation of the estimates.

SEC61 and SSH1 duplication promote faster doubling of the SEC72 silk production strain (FIG. 11, left). This effect may be due to decreasing the load of intracellularly accumulated silk. However, Δsec72 leads to a significant growth defect, which is partially restored by duplication of SSH1 but not SEC61 (FIG. 11, right).

### Example 12: SSH1 Duplication Improves Fermentation Performance of ΔSec72 Strain

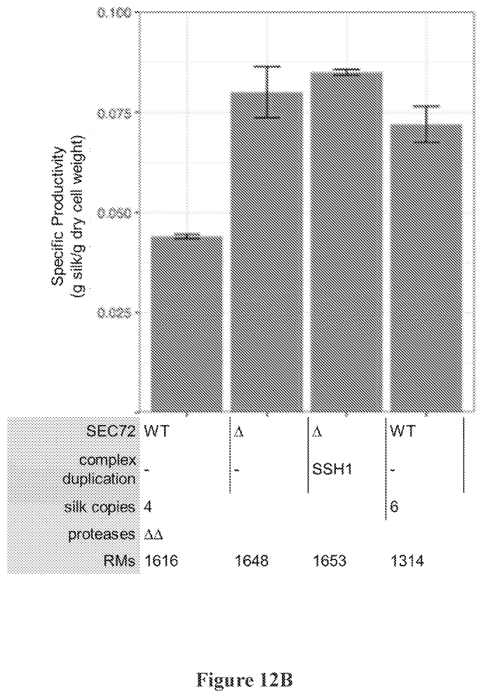

P. pastoris strains were modified according to Example 1 to comprise 4 copies or 6 copies of DNA cassettes comprising recombinant genes expressing 18B silk-like polypeptide. Δsec72 strains were prepared from recombinant cells expressing 4 copies of the DNA cassette using techniques described in Example 2. Some of the resulting P. pastoris (Δsec72) cells were also transformed with a vector for overexpression of the SSH1 translocon complex as described in Example 3. Secretion of 18B from each strain was measured using ELISA, with the results shown in FIG. 12A and FIG. 12B.

In FIG. 12A, each trajectory represents an independent batch-fed fermentation. Marked points are samples taken for analytes including the concentration of secreted silk protein in the broth (“18B titer”, y-axis). The x-axis is cumulative glucose fed, since the feed program is time-varying. Shading depicts the range of all runs of the same strain. The Δsec72 deletion strain shows highly variable performance. When SSH1 is duplicated in this strain (“2N SSH1”), performance improves and variability decreases. The Δsec72 2N SSH1 strain with only 4 silk expression cassettes secretes approaches the range of a reference strain with 6 silk expression cassettes.

Specific productivity (the ratio of product to biomass) was measured for each strain culture and shown in FIG. 12B. Δsec72 backgrounds grow more slowly and generate less biomass, elevating their metrics above that of the higher-expressing, highest-titer strain. Due to high variability in the Δsec72 strain, summarized data show only the top n=2 runs. Error bars are standard error of the mean.

### Other Embodiments

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### Sequences

TABLE 8
Forward (F) and Reverse (R) Primers for 5′ and
3′ homology arms (HA) targeting protease ORF

| | SEQ | |
|---|---|---|
| | ID | |
| Description | NO: | 5′ to 3′ Sequence |
| KO_PAS_chr4_0584 | 45 | TACTACAGGCTGGCTGTTCC |
| 5′ HA F | | |
| KO_PAS_chr4_0584 | 46 | CTCACTTAATCTTCTGTACTCTGAAGAA |
| 5′ HA R | | GTCCAACTGTTGAACGCC |
| KO_PAS_chr4_0584 | 47 | AGAAGTTGATTGAGACTTTCAACGAGGG |
| 3′ HA F | | TCCCCTTCAGCTACCTTT |
| KO_PAS_chr4_0584 | 48 | TCCCTGCTAAGCCCTAATCG |
| 3′ HA R | | |
| KO_PAS_chr3_1157 | 49 | CTCTGATTGCACGAGAAGGC |
| 5′ HA F | | |
| KO_PAS_chr3_1157 | 50 | CTCACTTAATCTTCTGTACTCTGAAGTG |
| 5′ HA R | | AAAGGCGATTGGAGTTGC |
| KO_PAS_chr3_1157 | 51 | AGAAGTTGATTGAGACTTTCAACGAGCT |
| 3′ HA F | | GGCTCTGCTTCTGGTACT |
| KO_PAS_chr3_1157 | 52 | GATGTTGAGGCGGGCATAAG |
| 3′ HA R | | |

TABLE 9
Forward and reverse primers for amplifying
modified sequences

| | SEQ | |
|---|---|---|
| | ID | |
| Description | NO: | Sequence (5′ to 3′) |
| KO_PAS_chr4_0584 | 53 | ACTTGTCAGGACGATACGGA |
| Verification F | | |
| KO_PAS_chr4_0584 | 54 | CCGGTCTCCCTGGAAATAGA |
| Verification R | | |
| KO_PAS_chr3_1157 | 55 | AGTTGTCCGTCATTAGCCCT |
| Verification F | | |
| KO_PAS_chr3_1157 | 56 | TGTTCCCTTTCGGCTAGACA |
| Verification R | | |

TABLE 10
sec72 KO vector

| | SEQ | |
|---|---|---|
| Descrip- | ID | |
| tion | NO: | Sequence (5′ to 3′) |
| SEC72 KO | 91 | ggcgcgccgtttaaaccctccaccagccatataccactacaacaccacagaagagaaagagctcatatcatccgtcatgagagagtac |
| full | | cagcacagaaatacagtcaagtaaaactagtatgcaagcattacgtaataatagcaactttatgacaaatcattccatttttttccac |
| plasmid | | tggagcgtgcactgcgtaaatcattctctttggaaggcaagggaagaacaacaaaatttttccttccgttatacaaacattgaatcat |
| | | gtctactgaacccacttttaaattggtccttgtcggtgatggtggtaccggtaaagtaagtgcaaattatttgatgagtcggataatg |
| | | ttttccgccccttagttcccctcatgattactaacaattcatagaccaccttcgttaagagacaccttactggagagttccgtaagaa |
| | | gtacattgctactttgggagtcgaagttcatcccttgtcattccacactaactgtggtcctatcacattcaacgtttgggacactgct |
| | | ggacaagagaagtttggtggactgagagatggttattacattaacggtgactgtggtatcatcatgttcgacgttacatcgagaatta |
| | | cttacaagaacgttccaaactggcaccgtgacttggtcagagtgtgtgagaacattccaattgtgctttgtggtaacaaggttgatgt |
| | | caaggaaagaaaggtcaaggctaagaccatcactttccacagaaagaagaacttgcaatactttgacatttctgccaagtccaactac |
| | | aactttgagaagccattcttgtggttagctagaaagttgtctggtgagccccaattagagttcgttgctgctcccgacttgcaagccc |
| | | cagaggttcaaattgatgccgatttaataaagaagtacgagcaagagaacgccgaggctgccgctatgccattgcctgatgaagatga |
| | | tgccgacttgtaagcttttacttacagtacattgagaaccatacatagggcacgtatcgtaagtttagttgtttgctgatgtaagcta |
| | | gtttgtttctgtagtgtttcgaggtcgcagagggatctctctagccttagacaaaaaaaaaaaggttgacacgttgatacactctctg |
| | | tttcatccgatctttcacctacgagtcccactcctcttcagagtacagaagattaagtgagagaattctaccgttcgtatagcataca |
| | | ttatacgaagttatttcagtaatgtcttgtttcttttgttgcagtggtgagccattttgacttcgtgaaagtttctttagaatagttg |
| | | tttccagaggccaaacattccacccgtagtaaagtgcaagcgtaggaagtccaagactggcataaatcaggtataagtgtcgagcact |
| | | ggcaggtgatcttctgaaagtttctactagcagataagatccagtagtcatgcatatggcaacaatgtaccgtgtggatctaagaacg |
| | | cgtcctactaaccttcgcattcgttggtccagtttgttgttatcgatcaacgtgacaaggttgtcgattccgcgtaagcatgcatacc |
| | | caaggacgcctgttgcaattccaagtgagccagttccaacaatctttgtaatattagagcacttcattgtgttgcgcttgaaagtaaa |
| | | atgcgaacaaattaagagataatctcgaaaccgcgacttcaaacgccaatatgatgtgcggcacacaataagcgttcatatccgctgg |
| | | gtgactttctcgctttaaaaaattatccgaaaaaatttttgacggctagctcagtcctaggtacgctagcattaaagaggagaaaatg |
| | | gctaaactgacctctgctgttccggttctgaccgctcgtgacgttgctggtgctgttgagttctggaccgaccgtctgggtttctctc |
| | | gtgacttcgttgaggacgacttcgctggtgttgttcgtgacgacgttaccctgttcatctctgctgttcaggaccaggttgttccgga |
| | | caacaccctggcttgggtttgggttcgtggtctggacgaactgtacgctgaatggtctgaagttgtttctaccaacttccgtgacgct |
| | | tctggtccggctatgaccgaaatcggtgaacagccgtggggtcgtgagttcgctctgcgtgacccggctggtaactgcgttcacttcg |
| | | ttgctgaagaacaggactaacacgtccgacggcggcccacgggtcccaggcctcggagatccgtcccccttttcctttgtcgatatca |
| | | tgtaattagttatgtcacgcttacattcacgccctccccccacatccgctctaaccgaaaaggaaggagttagacaacctgaagtcta |
| | | ggtccctatttatttttttatagttatgttagtattaagaacgttatttatatttcaaatttttcttttttttctgtacagacgcgtg |
| | | tacgcatgtaacattatactgaaaaccttgcttgagaaggttttgggacgctcgaaggctttaatttgcaagctataacttcgtatag |
| | | catacattataccttgttatgcggccgcagatctaacatccaaagacgaaaggttgaatgaaacctttttgccatccgacatccacag |
| | | gagagttatagagctgctatgcgtgtaaaaatagtttaaatcttcgtaaagtatgttagtccatgtaatttgctatgaatcgatacgc |
| | | taatctggatgctgaacggatgcttactggcatgcattattcattacccatctaagctgcgccacaacccagtaaattgcagtgaggg |
| | | aagcttccctgtaaccgtcctgtccctttagggaccatcgatccccaacgatcaaatcgcgatacatctatcaactgtccctttccat |
| | | ctatctatgcaaggtaatgacagactctgttaactctgatgattctgatctggaaatcatagaggtgactgagcctactccaaaagtg |
| | | gaccttttggcccccaatccagcatttaattttactgcccccataagcaacagtaacggcacaactccaataaggagaaaacttgatg |
| | | accaatccaactccaattcttttgccagactggaatcgttacgggaatcatcagtgaaaccacaagctagtacgttcaatagtagtag |
| | | gttcatcccccaagccgaccaattttccaataatcagaataatgaacttgataacaacaatggattcgccgactggatttctaagtcc |
| | | caacctgaatttccctttccacttaatgatggaccaaaaaagtccagcaatcaacctacaaactcaaattttgaagagatcatcgatt |
| | | taactgaagatatcgagataaatacatctgtccccgcatctacatcatcttctaccccagttccctccagcacacagaatcagagcca |
| | | tcatatagccaacaacaacacagcacaagatgcgcatatcttccaagggaaacgacctctccaatcatattcagatgatgaagacgaa |
| | | gatttgcaaattgtaggatccaatattgttcagcagcctctaggaattatgccaggaactttcaacgcccctgcaaacatactccatt |
| | | ttgacggttcaaaccagaatgaacaagccagatggctggacttgcggataaaagatttgttagataatcttcacaatcttcgagttca |
| | | tgctcagtcgaatattatggagatcaataggttcatttccactttggggcatttaaacagagaagtttaaaccctgcagggcgctcgg |
| | | tcgttcggctgcggcgagcggtatcagctcactcaaaggcggtaatacggttatccacagaatcaggggataacgcaggaaagaacat |
| | | gtgagcaaaaggccagcaaaaggccaggaaccgtaaaaaggccgcgttgctggcgtttttccataggctccgcccccctgacgagcat |
| | | cacaaaaatcgacgctcaagtcagaggtggcgaaacccgacaggactataaagataccaggcgtttccccctggaagctccctcgtgc |
| | | gctctcctgttccgaccctgccgcttaccggatacctgtccgcctttctcccttcgggaagcgtggcgctttctcatagctcacgctg |
| | | taggtatctcagttcggtgtaggtcgttcgctccaagctgggctgtgtgcacgaaccccccgttcagcccgaccgctgcgccttatcc |
| | | ggtaactatcgtcttgagtccaacccggtaagacacgacttatcgccactggcagcagccactggtaacaggattagcagagcgaggt |
| | | atgtaggcggtgctacagagttcttgaagtggtggcctaactacggctacactagaagaacagtatttggtatctgcgctctgctgaa |
| | | gccagttaccttcggaaaaagagttggtagctcttgatccggcaaacaaaccaccgctggtagcggtggtttttttgtttgcaagcag |
| | | cagattacgcgcagaaaaaaaggatctcaagaagatcctttgatcttttctacggggtctgacgctcagtggaacgaaaactcacgtt |
| | | aagggattttggtcatgagattatcaaaaaggatcttcacctagatccttttaaattaaaaatgaagttttaaatcaatctaaagtat |
| | | atatgagtaaacttggtctgacagttaccaatgcttaatcagtgaggcacctatctcagcgatctgtctatttcgttcatccatagtt |
| | | gcctgactccccgtcgtgtagataactacgatacgggagggcttaccatctggccccagtgctgcaatgataccgcgcgacccacgct |
| | | caccggctccagatttatcagcaataaaccagccagccggaagggccgagcgcagaagtggtcctgcaactttatccgcctccatcca |
| | | gtctattaattgttgccgggaagctagagtaagtagttcgccagttaatagtttgcgcaacgttgttgccattgctacaggcatcgtg |
| | | gtgtcacgctcgtcgtttggtatggcttcattcagctccggttcccaacgatcaaggcgagttacatgatcccccatgttgtgcaaaa |
| | | aagcggttagctccttcggtcctccgatcgttgtcagaagtaagttggccgcagtgttatcactcatggttatggcagcactgcataa |
| | | ttctcttactgtcatgccatccgtaagatgcttttctgtgactggtgagtactcaaccaagtcattctgagaatagtgtatgcggcga |
| | | ccgagttgctcttgcccggcgtcaatacgggataataccgcgccacatagcagaactttaaaagtgctcatcattggaaaacgttctt |
| | | cggggcgaaaactctcaaggatcttaccgctgttgagatccagttcgatgtaacccactcgtgcacccaactgatcttcagcatcttt |
| | | tactttcaccagcgtttctgggtgagcaaaaacaggaaggcaaaatgccgcaaaaaagggaataagggcgacacggaaatgttgaata |
| | | ctcatactcttcctttttcaatattattgaagcatttatcagggttattgtctcatgagcggatacatatttgaatgtatttagaaaa |
| | | ataaacaaataggggttccgcgcacatttccccgaaaagtgccacct |
| SEC72 | 92 | aaaccctccaccagccatataccactacaacaccacagaagagaaagagctcatatcatccgtcatgagagagtaccagcacagaaat |
| 5′ | | acagtcaagtaaaactagtatgcaagcattacgtaataatagcaactttatgacaaatcattccatttttttccactggagcgtgcac |
| homology | | tgcgtaaatcattctctttggaaggcaagggaagaacaacaaaatttttccttccgttatacaaacattgaatcatgtctactgaacc |
| arm | | cacttttaaattggtccttgtcggtgatggtggtaccggtaaagtaagtgcaaattatttgatgagtcggataatgttttccgcccct |
| | | tagttcccctcatgattactaacaattcatagaccaccttcgttaagagacaccttactggagagttccgtaagaagtacattgctac |
| | | tttgggagtcgaagttcatcccttgtcattccacactaactgtggtcctatcacattcaacgtttgggacactgctggacaagagaag |
| | | tttggtggactgagagatggttattacattaacggtgactgtggtatcatcatgttcgacgttacatcgagaattacttacaagaacg |
| | | ttccaaactggcaccgtgacttggtcagagtgtgtgagaacattccaattgtgctttgtggtaacaaggttgatgtcaaggaaagaaa |
| | | ggtcaaggctaagaccatcactttccacagaaagaagaacttgcaatactttgacatttctgccaagtccaactacaactttgagaag |
| | | ccattcttgtggttagctagaaagttgtctggtgagccccaattagagttcgttgctgctcccgacttgcaagccccagaggttcaaa |
| | | ttgatgccgatttaataaagaagtacgagcaagagaacgccgaggctgccgctatgccattgcctgatgaagatgatgccgacttgta |
| | | agcttttacttacagtacattgagaaccatacatagggcacgtatcgtaagtttagttgtttgctgatgtaagctagtttgtttctgt |
| | | agtgtttcgaggtcgcagagggatctctctagccttagacaaaaaaaaaaaggttgacacgttgatacactctctgtttcatccgatc |
| | | tttcacctacgag |
| SEC72 | 93 | ggagagttatagagctgctatgcgtgtaaaaatagtttaaatcttcgtaaagtatgttagtccatgtaatttgctatgaatcgatacg |
| 5′ | | ctaatctggatgctgaacggatgcttactggcatgcattattcattacccatctaagctgcgccacaacccagtaaattgcagtgagg |
| homology | | gaagcttccctgtaaccgtcctgtccctttagggaccatcgatccccaacgatcaaatcgcgatacatctatcaactgtccctttcca |
| arm | | tctatctatgcaaggtaatgacagactctgttaactctgatgattctgatctggaaatcatagaggtgactgagcctactccaaaagt |
| | | ggaccttttggcccccaatccagcatttaattttactgcccccataagcaacagtaacggcacaactccaataaggagaaaacttgat |
| | | gaccaatccaactccaattcttttgccagactggaatcgttacgggaatcatcagtgaaaccacaagctagtacgttcaatagtagta |
| | | ggttcatcccccaagccgaccaattttccaataatcagaataatgaacttgataacaacaatggattcgccgactggatttctaagtc |
| | | ccaacctgaatttccctttccacttaatgatggaccaaaaaagtccagcaatcaacctacaaactcaaattttgaagagatcatcgat |
| | | ttaactgaagatatcgagataaatacatctgtccccgcatctacatcatcttctaccccagttccctccagcacacagaatcagagcc |
| | | atcatatagccaacaacaacacagcacaagatgcgcatatcttccaagggaaacgacctctccaatcatattcagatgatgaagacga |
| | | agatttgcaaattgtaggatccaatattgttcagcagcctctaggaattatgccaggaactttcaacgcccctgcaaacatactccat |
| | | tttgacggttcaaaccagaatgaacaagccagatggctggacttgcggataaaagatttgttagataatcttcacaatcttcgagttc |
| | | atgctcagtcgaatattatggagatcaataggttcatttccactttggggcatttaaacagagaagttt |
| Yeast | 94 | ttcagtaatgtcttgtttcttttgttgcagtggtgagccattttgacttcgtgaaagtttctttagaatagttgtttccagaggccaa |
| selection | | acattccacccgtagtaaagtgcaagcgtaggaagtccaagactggcataaatcaggtataagtgtcgagcactggcaggtgatcttc |
| marker | | tgaaagtttctactagcagataagatccagtagtcatgcatatggcaacaatgtaccgtgtggatctaagaacgcgtcctactaacct |
| (Sh ble) | | tcgcattcgttggtccagtttgttgttatcgatcaacgtgacaaggttgtcgattccgcgtaagcatgcatacccaaggacgcctgtt |
| for SEC72 | | gcaattccaagtgagccagttccaacaatctttgtaatattagagcacttcattgtgttgcgcttgaaagtaaaatgcgaacaaatta |
| KO | | agagataatctcgaaaccgcgacttcaaacgccaatatgatgtgcggcacacaataagcgttcatatccgctgggtgactttctcgct |
| (nucleo- | | ttaaaaaattatccgaaaaaatttttgacggctagctcagtcctaggtacgctagcattaaagaggagaaaatggctaaactgacctc |
| tide | | tgctgttccggttctgaccgctcgtgacgttgctggtgctgttgagttctggaccgaccgtctgggtttctctcgtgacttcgttgag |
| sequence) | | gacgacttcgctggtgttgttcgtgacgacgttaccctgttcatctctgctgttcaggaccaggttgttccggacaacaccctggctt |
| | | gggtttgggttcgtggtctggacgaactgtacgctgaatggtctgaagttgtttctaccaacttccgtgacgcttctggtccggctat |
| | | gaccgaaatcggtgaacagccgtggggtcgtgagttcgctctgcgtgacccggctggtaactgcgttcacttcgttgctgaagaacag |
| | | gactaacacgtccgacggcggcccacgggtcccaggcctcggagatccgtcccccttttcctttgtcgatatcatgtaattagttatg |
| | | tcacgcttacattcacgccctccccccacatccgctctaaccgaaaaggaaggagttagacaacctgaagtctaggtccctatttatt |
| | | tttttatagttatgttagtattaagaacgttatttatatttcaaatttttcttttttttctgtacagacgcgtgtacgcatgtaacat |
| | | tatactgaaaaccttgcttgagaaggttttgggacgctcgaaggctttaatttgcaagct |
| Yeast | 95 | MAKLTSAVPVLTARDVAGAVEFWTDRLGFSRDFVEDDFAGVVRDDVTLFISAVQDQVVPDNTLAWVWVRGLDELYAEWSEVVSTNFRD |
| selection | | ASGPAMTEIGEQPWGREFALRDPAGNCVHFVAEEQD |
| marker | | |
| (Sh ble) | | |
| for SEC72 | | |
| KO | | |
| (protein | | |
| sequence) | | |

TABLE 11
SSH1 Complex Overexpression vector

| | SEQ | |
|---|---|---|
| Descrip- | ID | |
| tion | NO: | Sequence (5′ to 3′) |
| Full | 96 | ccagccaggacagaaatgcctcgacttcgctgctgcccaaggttgccgggtgacgcacaccgtggaaacggatgaaggcacgaacc |
| Plasmid | | cagtggacataagcctgttcggttcgtaagctgtaatgcaagtagcgtatgcgctcacgcaactggtccagaaccttgaccgaacg |
| | | cagcggtggtaacggcgcagtggcggttttcatggcttgttatgactgtttttttggggtacagtctatgcctcgggcatccaagc |
| | | agcaagcgcgttacgccgtgggtcgatgtttgatgttatggagcagcaacgatgttacgcagcagggcagtcgccctaaaacaaag |
| | | ttaaacatcatgagggaagcggtgatcgccgaagtatcgactcaactatcagaggtagttggcgtcatcgagcgccatctcgaacc |
| | | gacgttgctggccgtacatttgtacggctccgcagtggatggcggcctgaagccacacagtgatattgatttgctggttacggtga |
| | | ccgtaaggcttgatgaaacaacgcggcgagctttgatcaacgaccttttggaaacttcggcttcccctggagagagcgagattctc |
| | | cgcgctgtagaagtcaccattgttgtgcacgacgacatcattccgtggcgttatccagctaagcgcgaactgcaatttggagaatg |
| | | gcagcgcaatgacattcttgcaggtatcttcgagccagccacgatcgacattgatctggctatcttgctgacaaaagcaagagaac |
| | | atagcgttgccttggtaggtccagcggcggaggaactctttgatccggttcctgaacaggatctatttgaggcgctaaatgaaacc |
| | | ttaacgctatggaactcgccgcccgactgggctggtgatgagcgaaatgtagtgcttacgttgtcccgcatttggtacagcgcagt |
| | | aaccggcaaaatcgcgccgaaggatgtcgctgccgactgggcaatggagcgcctgccggcccagtatcagcccgtcatacttgaag |
| | | ctagacaggcttatcttggacaagaagaagatcgcttggcctcgcgcgcagatcagttggaagaatttgtccactacgtgaaaggc |
| | | gagatcaccaaggtagtcggcaaataacattactcgcatccattctcaggctgtctcgtctcgtctccaactttctgccgccaatc |
| | | tcctttccattcaaattctctacaacaaggcaggcttcgtccgttgatgtgaagtttgcaaaagccaaaccacgaaaaacaccatt |
| | | gtcaaaatggtagttgaaggcataaggcaaaggcaagctaaactttgtcataacgtctaaaagttgctctttttttatggcaaaag |
| | | ggatgttctttatcacaatagcagtgggaatgacatcttcatcatcattcgtatcatcaataccatcgagttctgaatccaaaggt |
| | | ttctgttgttgggataaggcatcagattctttgagcaggtcgtccttggcgtcctcctctagatgaaatttgccatcgttaggagg |
| | | tgagtattgggaaacgttgacttgttgctcaatccaaggatcaacgttgaccatcggattgccgttctgaggagacagccaggcat |
| | | tataccgtggttgaccggcgtttacgcgcagaggattgttgctggtgttgttgacagaagacgcatacccagacgctgcggatgac |
| | | atcgacgaaattgaagggcgacgttgcatacccatttgttgctgatccaagaaacttggtgtttcagtcaattgtctcaggtccat |
| | | attgatcaatgtgttcaactgtttcaatctggttccggagggtggctattacgacaagctgtggctactatctaagtgggagaagt |
| | | aacggaacacattgatgagacacaggaattacagggcgtgcatccaccaataacaattagtcgagatttcaaccaattacgtaagc |
| | | gctcaacccttttttcgaacacgtatcgagcaaagtccaggtgaaaccttcatccattatatccaaagtcgaccgaagctttaaca |
| | | acatccataatggtaagtgtccagtttgatgagtgcagaatggttccaagttttagaccagttactaatatttaaagtctacagca |
| | | attccaggaggacagagaacgttagctaaaagaagagcagcaaacttggataagaaacaggatgaaccaacctccgccagatctgc |
| | | cggtgctggaggttcttcgtctaccatgctaaagttgtacacagacgaggcccaaggtttgaaagttgatcctttaattgttcttg |
| | | ttcttgctgttggtttcattttcagtgtcattggtttgcacgttgttgctaagctgacaggaaagttgatcaactaagacctatat |
| | | ttaaacaggtttcatcatatctgtactatatttacaagtccactgcgtttaggtatatactaaagacattcaagaagcacatccac |
| | | aacttgtgcaagtcctgtcaaatgtactagatgcttttcagaacatcctgcggtttgaggagattcctgaatttcccagtcccaag |
| | | tctttctcttgtagaggtctttgagttcttgtgaatgctgaattggggttcttacctcaatttctattagtgggaaatgctttccc |
| | | acaattatttgcaatgggatcccggcaactttactttgcttcaacttatgtcccatactgaactttccgtcacggttgtcaacttg |
| | | aacgtcgaatgagctcagtatttcggtgacagtgtctagattctttttacttgatgttttattcgaaagtagcgtgatttgatatg |
| | | gtgcgataatttgtggccagggctgggagttcgtagacggaaacaaacgcagaatccaagcagacaagtggagagtggacaaattc |
| | | ttgatgttaattcgtaagatccttaacaaatctttagcaaaactgaaaagtgaagactacgatagggatttacttgaaaaatatat |
| | | tcaagttttgtcagaatacccactccatattaacgatattattgtccctagaactattacgtaccacttgtgtgatatttatactg |
| | | atgagttggaaaaggttatgtttagtgggcttcctggatttgaagaagaagaagattacgaggaagaagatgaagtctcggctcct |
| | | atcgaaaaaaaaaccagagatactgataattcagacgatgaggcctctgataccgatccagaaacgagtgacgaagaagacgaagg |
| | | tgagaatactgaaaacgagtcagaagaggaaccgatcaaactctctgcggaagaagaaacggctctgtggaaaacaaagatggaga |
| | | ttatccatgaaactcctatcgacaaattactctcaccttttgtctcattgaagaaagatacctcaaataaaccattaaaattgaaa |
| | | atccaagaagcagttcttgctgatccaagactcggcaaatggaaagttaaatcgtaccgaaagcctaaaccaaaaccaaaacctct |
| | | tcaggtgctacagaaacagttatacgaacagatcaaatataaaaagggtaaagcagtcacaggtgaagacgacgatgaactcaaag |
| | | acgaagacgaagatgacgatgacgaggtcatctcggagagtgaagctgataactctgatgaagaagaagatgaagaatggaatggc |
| | | tttggaactatctagaatacatatgtaaaaccatatcaggcaataacaatttctcgctattttgcatcccaacacctcgaccgacg |
| | | tgttcagttcgaccctttacctacagtgtctttaactcccatttggatctcctaaataacctacacaaatgtcccaaaaagtcacc |
| | | gacgtccctctggaatttgttaaggaaggttccaaattcatctctaaatgtactaaaccctctcagaaggagtacttaaagatagt |
| | | aagagctgttggagttgggtttttaatgatgggcgtggttggttacgttgtcaagctcattcatattccaatcagatatttgattg |
| | | tttaaaagtttaggtttgaatacaatgtgtatgcttaattatattcacttcgtttcattgatttttgctatccctgttgtgcgtta |
| | | atcatctctatcgtgatcctctcaagttgcacctcaaatagaagacaacttatggaggtgtactacccaatatcagtcttgacgtt |
| | | tctagtttcgttgtatgctgcataccaatttcagttcttccgtagtgttttgagcataggcttgtttacccgtcttctttacactc |
| | | tgatatgagcaccttatcaaacctcttatcacagctgaaaggagaaggcggtggtggttcttctggtcagaatcggcccagaactg |
| | | tggatcctgctgttgcaagattgaaagcagaaaggaagatggaaagagagaagcaagctcttaaagaggctcagcaagcccaggaa |
| | | gctcgggaaaggcgaagaattagtcatgctacccagataggtttgagaccaaccagtaaagctcccagcactgaaggtcctcaatc |
| | | gcactggaaacatcaaggtcgatgaccgcactaacagaaggagctaaactattcgaaaaggagattccttacattacagaattaga |
| | | gggtgatgtcgaaggaatgaaattcattatcaagggcgagggtactggtgacgctactaccggtacgattaaagcaaagtacatct |
| | | gtacaacaggtgaccttcctgttccgtgggctactctggtgagcactttgtcttatggagttcaatgttttgctaaatacccttcg |
| | | cacattaaagactttttcaaaagtgcaatgcctgagggctatactcaggagagaacaatatctttcgaaggagatggtgtgtataa |
| | | gactagggctatggtcacgtatgaaagaggatccatctacaatagagtaactttaactggtgaaaacttcaaaaaggacggtcaca |
| | | tccttagaaagaatgttgcctttcaatgcccaccatccatcttgtacattttgccagacacagttaacaatggtatcagagttgag |
| | | tttaaccaagcttatgacatagagggtgtcaccgaaaagttggttacaaaatgttcacagatgaatcgtcccctggcaggatcagc |
| | | tgccgtccatatcccacgttaccatcatatcacttatcataccaagctgtccaaagatcgtgatgagagaagggatcacatgtgtt |
| | | tggttgaagtggtaaaggccgtggatttggatacttaccaatgactgacctcctgccagcaatagtaagacaacacgcaaagtctc |
| | | tgaacgggtctttgagctgtctgtgtcatcaaacatatcttcatctgtagttgtattgttttcttttatcactaggagtccatcct |
| | | tctggatggctgtcttgcacttatttagccactccaaaaatgccgggtctgggagatgaccgcagcaccattgacaccatatcaga |
| | | gagtatcgagactcctctggtttccagtcttgcatgggaatttcataaatgtctcctatcttaccttgttccatcagtgtttgtag |
| | | ttccaccctcatctggtcgacaaatggtttaactggttccaataaatcaaccttatcacaaactttatgcagaaaatctcttgtca |
| | | cgcggccaataccggcaccaaagtctataccatatttgattttgtcaggatcgttagagaaacgactctttaactttcttaaaaag |
| | | gtcatcgatccaacaacatctgcttttggaaccgaagttgtctcaccgtacccacccaaaactccgtcgacagatgctgggacgct |
| | | gttccagtatttcagggcatcgtcatagttgatcaaactgtccacctgtttagggtcatccccattatgttctgtcattgtggtaa |
| | | aaatgggatacagtgatatattgaaggggaatggttataagagcctacctgagaaataaaattattatgcgccatccgacatccag |
| | | aaaaattgatgaaagattggctattgttgacggttcttgatcccaaaaaaaaaaaaaaacaagaaatgctgccgtcctagttttgc |
| | | ttcaaagaatggtttcgtgctatgccattcccaacccaaagagctgtcccatcccattaagttgtgctgactgattatgttgcaca |
| | | atccagtgtcgtgattacctccaacatcgcacgcgaatttcgccatggctgggaaacccaaattcttcggtcgtccatcaaactct |
| | | gaagtcatttcaacaccaaactcaacagctatcatagaaaaatatggcggggttgcgttttttagacattgcaagaccatttgtca |
| | | gctggatcccggaagttgaacttccttatgaaaactgggggttcgatgaaaagctgatttactcatttttcactgctgccatctat |
| | | ttgattctgtccctgcctatatacggtgtcaaatcctctgaagtcgtggacccagttccccatttgcgttctgccttagggagtga |
| | | gaagggaacattgctggagcttgggttactgcctgtgattacttcggcatttatcttgcagttgttggctggttggaaagttttca |
| | | aagtaaactttgatctggttagtgacagaatattgttccaaactttgcaaaagatcacttcagtcgttatcagcatcgtatatgct |
| | | gttcttctcacattttgtgactactttactccaggtgtgtccactgataacgtcttgtggtcccaatttctgatcatcttacagat |
| | | agtggtggtcaacttcttggttactctactcgttgaagtcattgacaaggattacggattttcttcaggagctctattgttgcttg |
| | | cggtttattccgccaccaacttcgtttttggcacgattggtcttagcaccgtcaacacctccagatcgaacgaatctattggtgct |
| | | ctgattcaattattccgcaatttgagctctaaaccaattggtgttgccatatatgactccttcttcagagtaaaccttcctaactt |
| | | gactcaattttatctggggattgccattatttgtgtttgtctgttcttgaataatgcaagatacgaagtaccaattaagccaaaca |
| | | aggttcgtgccatggcctcagcttacccaatcaagctacttttcaatggttctttgccacttctgtacacgtggactgtgctgtac |
| | | aacttgaaccttattggtttctttgtcttcaagcttaccaacttttctcttttagggaacttcaaagtggacccattcggcaacaa |
| | | ctactacgaaattacatctggactgctgtatttattgactcctactttcaacgctgaagctggacttttacccaatgttgctaagc |
| | | catttgttttcattgccttctatgttggtgttagcactttctttgctagatcgtggtccaacattaacgggtcgtcaggcaaggac |
| | | attgccaagtttttcaaggctcaaggaatctcattgttaggaaaaagagatgcctctgtgtctaaagagtttaacaccctagttcc |
| | | tgttgcttctgcctctggagctttcctattgtcttttccagttgccgtcgctgagttattgggtggctctggtgttccaacctcta |
| | | tcggaatcggtcttttgagtggtttggctattttggaaactgttttgcaagaatggcaacagtctggaggtgcctcacagttctcc |
| | | caatacttccagacttcttaggtttagaaatccttgaagactatccagacattcacccgcacctcaatttaccttctacatacatc |
| | | acatattctatagaggagagttccattgctcgtactgaaccccacaccactcttctttataccctacaaactcttcgtccaactca |
| | | atggcgtcattcgtgtcggtatagacaataatggtaccccagtccatttcaaagttgtctttttcgatatccatgattaatttggg |
| | | cattatttgaagttcgaactgttttcctggcactttagctttgatgatcgtttgatatatttcatccttggagttatacagtagtg |
| | | gctttcctcccaggtggtatcgtaaaacctgggaaggattgtgctcaagagccaactccctttacaacctcactcaagtccgttag |
| | | agggcgcgccgcacatgaagctgtacatggaaggcacggtgaataaccaccacttcaaatgcaccagcgagggtgagggtaaaccg |
| | | tatgaaggcacccaaacgatgcgtatcaaagttgttgagggtggcccgttgccgtttgcgttcgacattttagcgacgagctttat |
| | | gtatggctctcgtacgtttatcaagtacccgaagggtattccggactttttcaaacaatcttttccagagggtttcacctgggagc |
| | | gcgtgactcgctacgaagatggcggcgtcgtgaccgcaacgcaggatacctccctggaagatggctgcctggtctaccacgttcag |
| | | gtccgtggtgtcaatttcccgagcaatggtccggttatgcagaagaaaaccctgggttgggaaccgaacaccgagatgttgtatcc |
| | | tgcagatggtggcctggaaggtcgcagcgacatggcattgaaactggtcggtggcggccatctgagctgtagcttcgtgaccacgt |
| | | atcgttcgaagaaaacggtcggtaacatcaaaatgccgggtattcacgcggttgaccaccgtctggtgcgcattaaagaagccgac |
| | | aaagagacttacgtggagcaacatgaagtagccgttgcgaaatttgctggtttgggcggtggtatggacgaactgtacagttcctt |
| | | atcatctggcgaatcggacccacaagagcactgggttccgttttacattccaggaagagtttcagtaatgtcttgtttcttttgtt |
| | | gcagtggtgagccattttgacttcgtgaaagtttctttagaatagttgtttccagaggccaaacattccacccgtagtaaagtgca |
| | | agcgtaggaagaccaagactggcataaatcaggtataagtgtcgagcactggcaggtgatcttctgaaagtttctactagcagata |
| | | agatccagtagtcatgcatatggcaacaatgtaccgtgtggatctaagaacgcgtcctactaaccttcgcattcgttggtccagtt |
| | | tgttgttatcgatcaacgtgacaaggttgtcgattccgcgtaagcatgcatacccaaggacgcctgttgcaattccaagtgagcca |
| | | gttccaacaatctttgtaatattagagcacttcattgtgttgcgcttgaaagtaaaatgcgaacaaattaagagataatctcgaaa |
| | | ccgcgacttcaaacgccaatatgatgtgcggcacacaataagcgttcatatccgctgggtgactttctcgctttaaaaaattatcc |
| | | gaaaaaatttttgacggctagctcagtcctaggtacgctagcattaaagaggagaaaatgactactcttgatgacacagcctacag |
| | | atataggacatcagttccgggtgacgcagaggctatcgaagccttggacggttcattcactactgatacggtgtttagagtcaccg |
| | | ctacaggtgatggcttcaccttgagagaggttcctgtagacccacccttaacgaaagttttccctgatgacgaatcggatgacgag |
| | | tctgatgctggtgaggacggtgaccctgattccagaacatttgtcgcatacggagatgatggtgacctggctggctttgttgtggt |
| | | gtcctacagcggatggaatcgtagactcacagttgaggacatcgaagttgcacctgaacatcgtggtcacggtgttggtcgtgcac |
| | | tgatgggactggcaacagagtttgctagagaaagaggagccggacatttgtggttagaagtgaccaatgtcaacgctcctgctatt |
| | | cacgcatataggcgaatgggtttcactttgtgcggtcttgatactgctttgtatgacggaactgcttctgatggtgaacaagctct |
| | | ttacatgagtatgccatgtccatagcacgtccgacggcggcccacgggtcccaggcctcggagatccgtcccccttttcctttgtc |
| | | gatatcatgtaattagttatgtcacgcttacattcacgccctccccccacatccgctctaaccgaaaaggaaggagttagacaacc |
| | | tgaagtctaggtccctatttatttttttatagttatgttagtattaagaacgttatttatatttcaaatttttcttttttttctgt |
| | | acagacgcgtgtacgcatgtaacattatactgaaaaccttgcttgagaaggttttgggacgctcgaaggctttaatttgcaagctc |
| | | cgaataacttcgtatagcatacattataccttgttattacagcggccgcaaatattttatctgattaataagatgatcttcttgag |
| | | atcgttttggtctgcgcgtaatctcttgctctgaaaacgaaaaaaccgccttgcagggcggtttttcgaaggttctctgagctacc |
| | | aactctttgaaccgaggtaactggcttggaggagcgcagtcaccaaaacttgtcctttcagtttagccttaaccggcgcatgactt |
| | | caagactaactcctctaaatcaattaccagtggctgctgccagtggtgcttttgcatgtctttccgggttggactcaagacgatag |
| | | ttaccggataaggcgcagcggtcggactgaacggggggttcgtgcatacagtccagcttggagcgaactgcctacccggaactgag |
| | | tgtcaggcgtggaatgagacaaacgcggccataacagcggaatgacaccggtaaaccgaaaggcaggaacaggagagcgcacgagg |
| | | gagccgccagggggaaacgcctggtatctttatagtcctgtcgggtttcgccaccactgatttgagcgtcagatttcgtgatgctt |
| | | gtcaggggggcggagcctatggaaaaacggctttgccgcggccctctcacttccctgttaagtatcttcctggcatcttccaggaa |
| | | atctccgccccgttcgtaagccatttccgctcgccgcagtcgaacgaccgagcgtagcgagtcagtgagcgaggaagcggaatata |
| | | tcctgtatcacatattctgctgacgcaccggtgcagccttttttctcctgccacatgaagcacttcactgacaccctcatcagtgc |
| | | caacatagtaagccagtatacactccgctagcgctgatgtccggcggtgcgacgtc |
| SBH2 | 97 | caactttctgccgccaatctcctttccattcaaattctctacaacaaggcaggcttcgtccgttgatgtgaagtttgcaaaagcca |
| Promoter | | aaccacgaaaaacaccattgtcaaaatggtagttgaaggcataaggcaaaggcaagctaaactttgtcataacgtctaaaagttgc |
| | | tctttttttatggcaaaagggatgttctttatcacaatagcagtgggaatgacatcttcatcatcattcgtatcatcaataccatc |
| | | gagttctgaatccaaaggtttctgttgttgggataaggcatcagattctttgagcaggtcgtccttggcgtcctcctctagatgaa |
| | | atttgccatcgttaggaggtgagtattgggaaacgttgacttgttgctcaatccaaggatcaacgttgaccatcggattgccgttc |
| | | tgaggagacagccaggcattataccgtggttgaccggcgtttacgcgcagaggattgttgctggtgttgttgacagaagacgcata |
| | | cccagacgctgcggatgacatcgacgaaattgaagggcgacgttgcatacccatttgttgctgatccaagaaacttggtgtttcag |
| | | tcaattgtctcaggtccatattgatcaatgtgttcaactgtttcaatctggttccggagggtggctattacgacaagctgtggcta |
| | | ctatctaagtgggagaagtaacggaacacattgatgagacacaggaattacagggcgtgcatccaccaataacaattagtcgagat |
| | | ttcaaccaattacgtaagcgctcaacccttttttcgaacacgtatcgagcaaagtccaggtgaaaccttcatccattatatccaaa |
| | | gtcgaccgaagctttaacaacatccata |
| SSS1 | 98 | agacaagtggagagtggacaaattcttgatgttaattcgtaagatccttaacaaatctttagcaaaactgaaaagtgaagactacg |
| Promoter | | atagggatttacttgaaaaatatattcaagttttgtcagaatacccactccatattaacgatattattgtccctagaactattacg |
| | | taccacttgtgtgatatttatactgatgagttggaaaaggttatgtttagtgggcttcctggatttgaagaagaagaagattacga |
| | | ggaagaagatgaagtctcggctcctatcgaaaaaaaaaccagagatactgataattcagacgatgaggcctctgataccgatccag |
| | | aaacgagtgacgaagaagacgaaggtgagaatactgaaaacgagtcagaagaggaaccgatcaaactctctgcggaagaagaaacg |
| | | gctctgtggaaaacaaagatggagattatccatgaaactcctatcgacaaattactctcaccttttgtctcattgaagaaagatac |
| | | ctcaaataaaccattaaaattgaaaatccaagaagcagttcttgctgatccaagactcggcaaatggaaagttaaatcgtaccgaa |
| | | agcctaaaccaaaaccaaaacctcttcaggtgctacagaaacagttatacgaacagatcaaatataaaaagggtaaagcagtcaca |
| | | ggtgaagacgacgatgaactcaaagacgaagacgaagatgacgatgacgaggtcatctcggagagtgaagctgataactctgatga |
| | | agaagaagatgaagaatggaatggctttggaactatctagaatacatatgtaaaaccatatcaggcaataacaatttctcgctatt |
| | | ttgcatcccaacacctcgaccgacgtgttcagttcgaccctttacctacagtgtctttaactcccatttggatctcctaaataacc |
| | | tacacaa |
| SSH1 | 99 | tctgaacgggtctttgagctgtctgtgtcatcaaacatatcttcatctgtagttgtattgttttcttttatcactaggagtccatc |
| Promoter | | cttctggatggctgtcttgcacttatttagccactccaaaaatgccgggtctgggagatgaccgcagcaccattgacaccatatca |
| | | gagagtatcgagactcctctggtttccagtcttgcatgggaatttcataaatgtctcctatcttaccttgttccatcagtgtttgt |
| | | agttccaccctcatctggtcgacaaatggtttaactggttccaataaatcaaccttatcacaaactttatgcagaaaatctcttgt |
| | | cacgcggccaataccggcaccaaagtctataccatatttgattttgtcaggatcgttagagaaacgactctttaactttcttaaaa |
| | | aggtcatcgatccaacaacatctgcttttggaaccgaagttgtctcaccgtacccacccaaaactccgtcgacagatgctgggacg |
| | | ctgttccagtatttcagggcatcgtcatagttgatcaaactgtccacctgtttagggtcatccccattatgttctgtcattgtggt |
| | | aaaaatgggatacagtgatatattgaaggggaatggttataagagcctacctgagaaataaaattattatgcgccatccgacatcc |
| | | agaaaaattgatgaaagattggctattgttgacggttcttgatcccaaaaaaaaaaaaaaacaagaaatgctgccgtcctagtttt |
| | | gcttcaaagaatggtttcgtgctatgccattcccaacccaaagagctgtcccatcccattaagttgtgctgactgattatgttgca |
| | | caatccagtgtcgtgattacctccaacatcgcacgcgaatttcgccatggctgggaaacccaaattcttcggtcgtccatcaaact |
| | | ctgaagtcatttcaacaccaaactcaacagctatcatagaaaaat |
| SBH2 | 100 | gacctatatttaaacaggtttcatcatatctgtactatatttacaagtccactgcgtttaggtatatactaaagacattcaagaag |
| Terminator | | cacatccacaacttgtgcaagtcctgtcaaatgtactagatgcttttcagaacatcctgcggtttgaggagattcctgaatttccc |
| | | agtcccaagtctttctcttgtagaggtctttgagttcttgtgaatgctgaattggggttcttacctcaatttctattagtgggaaa |
| | | tgctttcccacaattatttgcaatgggatcccggcaactttactttgcttcaacttatgtcccatactgaactttccgtcacggtt |
| | | gtcaacttgaacgtcgaatgagctcagtatttcggtgacagtgtctagattctttttacttgatgttttattcgaaagtagcgtga |
| | | tttgatatggtgcgataatttgtggccagg |
| SSS1 | 101 | aagtttaggtttgaatacaatgtgtatgcttaattatattcacttcgtttcattgatttttgctatccctgttgtgcgttaatcat |
| Terminator | | ctctatcgtgatcctctcaagttgcacctcaaatagaagacaacttatggaggtgtactacccaatatcagtcttgacgtttctag |
| | | tttcgttgtatgctgcataccaatttcagttcttccgtagtgttttgagcataggcttgtttacccgtcttctttacactctgata |
| | | tgagcaccttatcaaacctcttatcacagctgaaaggagaaggcggtggtggttcttctggtcagaatcggcccagaactgtggat |
| | | cctgctgttgcaagattgaaagcagaaaggaagatggaaagagagaagcaagctcttaaagaggctcagcaagcccaggaagctcg |
| | | ggaaaggcgaagaattagtcatgctacccagataggtttgagaccaaccagtaaagctccca |
| SSH1 | 102 | gtttagaaatccttgaagactatccagacattcacccgcacctcaatttaccttctacatacatcacatattctatagaggagagt |
| Terminator | | tccattgctcgtactgaaccccacaccactcttctttataccctacaaactcttcgtccaactcaatggcgtcattcgtgtcggta |
| | | tagacaataatggtaccccagtccatttcaaagttgtctttttcgatatccatgattaatttgggcattatttgaagttcgaactg |
| | | ttttcctggcactttagctttgatgatcgtttgatatatttcatccttggagttatacagtagtggctttcctcccaggtggtatc |
| | | gtaaaacctgggaaggattgtgctcaa |
| Yeast | 103 | ttcagtaatgtcttgtttcttttgttgcagtggtgagccattttgacttcgtgaaagtttctttagaatagttgtttccagaggcc |
| Selection | | aaacattccacccgtagtaaagtgcaagcgtaggaagaccaagactggcataaatcaggtataagtgtcgagcactggcaggtgat |
| Marker | | cttctgaaagtttctactagcagataagatccagtagtcatgcatatggcaacaatgtaccgtgtggatctaagaacgcgtcctac |
| (Nat) for | | taaccttcgcattcgttggtccagtttgttgttatcgatcaacgtgacaaggttgtcgattccgcgtaagcatgcatacccaagga |
| SSH1 | | cgcctgttgcaattccaagtgagccagttccaacaatctttgtaatattagagcacttcattgtgttgcgcttgaaagtaaaatgc |
| overex- | | gaacaaattaagagataatctcgaaaccgcgacttcaaacgccaatatgatgtgcggcacacaataagcgttcatatccgctgggt |
| pression | | gactttctcgctttaaaaaattatccgaaaaaatttttgacggctagctcagtcctaggtacgctagcattaaagaggagaaaatg |
| genes | | actactcttgatgacacagcctacagatataggacatcagttccgggtgacgcagaggctatcgaagccttggacggttcattcac |
| (Nucleo- | | tactgatacggtgtttagagtcaccgctacaggtgatggcttcaccttgagagaggttcctgtagacccacccttaacgaaagttt |
| tide) | | tccctgatgacgaatcggatgacgagtctgatgctggtgaggacggtgaccctgattccagaacatttgtcgcatacggagatgat |
| | | ggtgacctggctggctttgttgtggtgtcctacagcggatggaatcgtagactcacagttgaggacatcgaagttgcacctgaaca |
| | | tcgtggtcacggtgttggtcgtgcactgatgggactggcaacagagtttgctagagaaagaggagccggacatttgtggttagaag |
| | | tgaccaatgtcaacgctcctgctattcacgcatataggcgaatgggtttcactttgtgcggtcttgatactgctttgtatgacgga |
| | | actgcttctgatggtgaacaagctctttacatgagtatgccatgtccatagcacgtccgacggcggcccacgggtcccaggcctcg |
| | | gagatccgtcccccttttcctttgtcgatatcatgtaattagttatgtcacgcttacattcacgccctccccccacatccgctcta |
| | | accgaaaaggaaggagttagacaacctgaagtctaggtccctatttatttttttatagttatgttagtattaagaacgttatttat |
| | | atttcaaatttttcttttttttctgtacagacgcgtgtacgcatgtaacattatactgaaaaccttgcttgagaaggttttgggac |
| | | gctcgaaggctttaatttgcaagct |
| Y Yeast | 104 | MTTLDDTAYRYRTSVPGDAEAIEALDGSFTTDTVFRVTATGDGFTLREVPVDPPLTKVFPDDESDDESDAGEDGDPDSRTFVAYGD |
| Selection | | DGDLAGFVVVSYSGWNRRLTVEDIEVAPEHRGHGVGRALMGLATEFARERGAGHLWLEVTNVNAPAIHAYRRMGFTLCGLDTALYD |
| Marker | | GTASDGEQALYMSMPCP |
| (Nat) for | | |
| SSH1 | | |
| overex- | | |
| pression | | |
| genes | | |
| (Amino | | |
| Acid) | | |

TABLE 12
Zeocin Cassette with HA arms for KU70 deletion in P. pastoris

| | SEQ | |
|---|---|---|
| Descrip- | ID | |
| tion | NO: | 5′ to 3′ Sequence |
| Plasmid | 57 | ggagttgaatcacatcttactggatagcgagctttttgacgaagtgaaaatttctaattttaaacaagaggaaggggtca |
| sequence | | aaaacggagatatcttatacttggaaaaagagatgacaatcagtgatttcatcaattttgtatctagttggccttctgtg |
| | | ttttcgtggaagcagcaacgaggaaaggagggtatcctagatgatttttacaacgaactgaacgactgctttgagggggg |
| | | taacatgaaagtaatatggaactccgtcctagtatttgccaggaggaagcaaagggttgtataggctttagtacttatag |
| | | aggaaacggggttacgtgcaagcgcgcatgcctgagctttgaggggggggactttcacatctcttcttctcacacttagc |
| | | cctaacacagagaataataaaaagcattgcaagatgagtgttgtcagcaagcaatacgacatccacgaaggcattatctt |
| | | tgtaattgaattgaccccggagcttcacgcgccggcttcagaagggaaatctcagctccagatcatcttagagaatgtca |
| | | gtgaggttatttctgagctaatcattaccttgcccggtacaggaatagggtgttaccttattaattacgacggtggtcaa |
| | | aacgacgaaatttaccccatttttgagttacaagacctgaatttggaaatgatgaaacaattgtaccaagtcttggagga |
| | | ccatgtaagtgggcttaatcctctcgagaagcaattcccaattgaacacagtaaaccgttatcagccactctgttctttc |
| | | acttaaggtctcttttttacatggcgaagactcataagcgtactggaagacattacaacttgaaaaagattttcttgttc |
| | | actaataacgataaaccttacaatggaaactctcagctgagagttcccttgaagaaaaccctggctgattacaatgacgt |
| | | agacattactttgattccgtttcttctgaacaagccttcaggtgtcaagtttgacaagacggaatactcagaaattttgt |
| | | tctatgataaagatgcttgttcgatgtcaattgaggagatccgccaacgaatttctagacataaggagatcaagcgggtt |
| | | tacttcacctgtcctttgaaaatcgcaaataacttgtgcatttctgtgaaaggttattctatgttttatcatgaaactcc |
| | | aaggaagatcaaatttgtcgtcaatgagggttcaactttcaaagatgtggagacaaaatctcagtttgtcgatccaacat |
| | | ccggaaaagagttttccagtgaacagctgatcaaagcatatcctctaggtgccgatgcttacattcctttaaactcagag |
| | | caagtcaaaacaataaatcgatttaatgatatcatcaatatcccctctttggaaattctaggtttcagggatatatctaa |
| | | ttggttgccacagtatcagtttggcaaagcatcgtttttatcccctaataactatggtgattttacacattcgcagagaa |
| | | catttagttgtcttcagtaatgtcttgtttcttttgttgcagtggtgagccattttgacttcgtgaaagtttctttagaa |
| | | tagttgtttccagaggccaaacattccacccgtagtaaagtgcaagcgtaggaagaccaagactggcataaatcaggtat |
| | | aagtgtcgagcactggcaggtgatcttctgaaagtttctactagcagataagatccagtagtcatgcatatggcaacaat |
| | | gtaccgtgtggatctaagaacgcgtcctactaaccttcgcattcgttggtccagtttgttgttatcgatcaacgtgacaa |
| | | ggttgtcgattccgcgtaagcatgcatacccaaggacgcctgttgcaattccaagtgagccagttccaacaatctttgta |
| | | atattagagcacttcattgtgttgcgcttgaaagtaaaatgcgaacaaattaagagataatctcgaaaccgcgacttcaa |
| | | acgccaatatgatgtgcggcacacaataagcgttcatatccgctgggtgactttctcgctttaaaaaattatccgaaaaa |
| | | atttttgacggctagctcagtcctaggtacgctagcattaaagaggagaaaatggctaaactgacctctgctgttccggt |
| | | tctgaccgctcgtgacgttgctggtgctgttgagttctggaccgaccgtctgggtttctctcgtgacttcgttgaagacg |
| | | acttcgctggtgttgttcgtgacgacgttaccctgttcatctctgctgttcaggaccaggttgttccggacaacaccctg |
| | | gcttgggtttgggttcgtggtctggacgaactgtacgctgaatggtctgaagttgtttctaccaacttccgtgacgcttc |
| | | tggtccggctatgaccgaaatcggtgaacagccgtggggtcgtgagttcgctctgcgtgacccggctggtaactgcgttc |
| | | acttcgttgctgaagaacaggactaacacgtccgacggcggcccacgggtcccaggcctcggagatccgtcccccttttc |
| | | ctttgtcgatatcatgtaattagttatgtcacgcttacattcacgccctccccccacatccgctctaaccgaaaaggaag |
| | | gagttagacaacctgaagtctaggtccctatttatttttttatagttatgttagtattaagaacgttatttatatttcaa |
| | | atttttcttttttttctgtacagacgcgtgtacgcatgtaacattatactgaaaaccttgcttgagaaggttttgggacg |
| | | ctcgaaggctttaatttgcaagctgtattagtttcacttttcagcaacctggtcggaaagatccacatcaagaatggata |
| | | ccaaccccaagagtatgaaaatccttccctacaatggcacttcaaaatgttacgtgacgattaccttcaattggaacacg |
| | | atatcgacatcagtgacccccttgagaaacaaaagtacataaacagcctcgatgagacaaaaaccaagatcatgaaacta |
| | | cgggactatgtcaaggaaactgccgatgatgacgacccttcacggcttgccaacactctcaaagagctcaaccaagagct |
| | | gaacaaaatttccaactttgatatcatcgccaataagaagccaaagacccccacgacagtagaccctgttcctactgatg |
| | | atgacatcatcaacgcctggaaggcaggaactctgaacggtttcaaggtggatcaattacgaaaatacgtaaggtcacga |
| | | aacaactttctggagacggcctccaaaaaggcagatctcatcgccaacattgacaagtactttcagcagaagttcaaaga |
| | | gactaaggcctgattcgtgttccttactttttcctcgcaacgtgtttttttcccaccacattgcctatgttgtaatgcaa |
| | | tgcagatgctggcccagtttttgacgattctcgaaaattggcattttcgtcgatgccattggccaaactgaaaattcaag |
| | | acaaaatagattggattttatctgcaacgtcttccacctacacaaccactctacaaacttcagacaaacatgtttataaa |
| | | agcagctactagatccaaaatgacaagttcgttattctctactacgtttgttgtggcatttggattggtggctagcaaca |
| | | acctcttgccatgtcctgttgaccactctatgaataacgagactccgcaagaattgaaaccattgcaggctgaatcttct |
| | | actagaaagttgaactcttccgcttaagtcaaataaaactactgacacagatgatgcacagaaacaacggatcacgctct |
| | | tgactgattagtcccgtcattttggttctcattttcttcacagtcacctatcaatgtatgatcacctggaaggatttccc |
| | | tacgatacttcaaatcttttacttgataatattactcattatggctcaggaatgcagactgcctgattcaagacgctgct |
| | | cttcttatttaacacttgtacactaaccccatggaagccagggaagggaataaccatctctctggtaataaatcggtctt |
| | | tatttatgcatagaaaaggaatctattatatttcgttcatttggcactctgctaactgtagattaacgggtctcgtaaat |
| | | tcaaaatcttcttccgatcaaaccggggtgaaatattacttctcgtgcatagctaattttcaaataaccgtcctaaaatg |
| | | aacggtcatttacctggactctcttgccaaatgggcaacaaaacataaagctgatcagaacgtaactagtctctcggaat |
| | | ccat |
| HA F | 58 | ggagttgaatcacatcttactg |
| KU70 HA 1 | 59 | gacaactaaatgttctctgcgaatgtgtaaaatcaccatagttattaggggataaaaacgatgctttgccaaactgatac |
| | | tgtggcaaccaattagatatatccctgaaacctagaatttccaaagaggggatattgatgatatcattaaatcgatttat |
| | | tgttttgacttgctctgagtttaaaggaatgtaagcatcggcacctagaggatatgctttgatcagctgttcactggaaa |
| | | actcttttccggatgttggatcgacaaactgagattttgtctccacatctttgaaagttgaaccctcattgacgacaaat |
| | | ttgatcttccttggagtttcatgataaaacatagaataacctttcacagaaatgcacaagttatttgcgattttcaaagg |
| | | acaggtgaagtaaacccgcttgatctccttatgtctagaaattcgttggcggatctcctcaattgacatcgaacaagcat |
| | | ctttatcatagaacaaaatttctgagtattccgtcttgtcaaacttgacacctgaaggcttgttcagaagaaacggaatc |
| | | aaagtaatgtctacgtcattgtaatcagccagggttttcttcaagggaactctcagctgagagtttccattgtaaggttt |
| | | atcgttattagtgaacaagaaaatctttttcaagttgtaatgtcttccagtacgcttatgagtcttcgccatgtaaaaaa |
| | | gagaccttaagtgaaagaacagagtggctgataacggtttactgtgttcaattgggaattgcttctcgagaggattaagc |
| | | ccacttacatggtcctccaagacttggtacaattgtttcatcatttccaaattcaggtcttgtaactcaaaaatggggta |
| | | aatttcgtcgttttgaccaccgtcgtaattaataaggtaacaccctattcctgtaccgggcaaggtaatgattagctcag |
| | | aaataacctcactgacattctctaagatgatctggagctgagatttcccttctgaagccggcgcgtgaagctccggggtc |
| | | aattcaattacaaagataatgccttcgtggatgtcgtattgcttgctgacaacactcat |
| KU70 HA 2 | 60 | tcaggccttagtctctttgaacttctgctgaaagtacttgtcaatgttggcgatgagatctgcctttttggaggccgtct |
| | | ccagaaagttgtttcgtgaccttacgtattttcgtaattgatccaccttgaaaccgttcagagttcctgccttccaggcg |
| | | ttgatgatgtcatcatcagtaggaacagggtctactgtcgtgggggtctttggcttcttattggcgatgatatcaaagtt |
| | | ggaaattttgttcagctcttggttgagctctttgagagtgttggcaagccgtgaagggtcgtcatcatcggcagtttcct |
| | | tgacatagtcccgtagtttcatgatcttggtttttgtctcatcgaggctgtttatgtacttttgtttctcaagggggtca |
| | | ctgatgtcgatatcgtgttccaattgaaggtaatcgtcacgtaacattttgaagtgccattgtagggaaggattttcata |
| | | ctcttggggttggtatccattcttgatgtggatctttccgaccaggttgctgaaaagtgaaactaatac |
| pILV5 | 61 | ttcagtaatgtcttgtttcttttgttgcagtggtgagccattttgacttcgtgaaagtttctttagaatagttgtttcca |
| | | gaggccaaacattccacccgtagtaaagtgcaagcgtaggaagaccaagactggcataaatcaggtataagtgtcgagca |
| | | ctggcaggtgatcttctgaaagtttctactagcagataagatccagtagtcatgcatatggcaacaatgtaccgtgtgga |
| | | tctaagaacgcgtcctactaaccttcgcattcgttggtccagtttgttgttatcgatcaacgtgacaaggttgtcgattc |
| | | cgcgtaagcatgcatacccaaggacgcctgttgcaattccaagtgagccagttccaacaatctttgtaatattagagcac |
| | | ttcattgtgttgcgcttgaaagtaaaatgcgaacaaattaagagataatctcgaaaccgcgacttcaaacgccaatatga |
| | | tgtgcggcacacaataagcgttcatatccgctgggtgactttctcgctttaaaaaattatccgaaaaaattt |
| RM2734; | 62 | cagaggccaaacattccacc |
| testR | | |
| pproRBS | 63 | ttaaagaggagaaa |
| Sh ble | 64 | atggctaaactgacctctgctgttccggttctgaccgctcgtgacgttgctggtgctgttgagttctggaccgaccgtct |
| (codon | | gggtttctctcgtgacttcgttgaagacgacttcgctggtgttgttcgtgacgacgttaccctgttcatctctgctgttc |
| optimized) | | aggaccaggttgttccggacaacaccctggcttgggtttgggttcgtggtctggacgaactgtacgctgaatggtctgaa |
| | | gttgtttctaccaacttccgtgacgcttctggtccggctatgaccgaaatcggtgaacagccgtggggtcgtgagttcgc |
| | | tctgcgtgacccggctggtaactgcgttcacttcgttgctgaagaacaggactaa |
| CYC1 | 65 | cacgtccgacggcggcccacgggtcccaggcctcggagatccgtcccccttttcctttgtcgatatcatgtaattagtta |
| terminator | | tgtcacgcttacattcacgccctccccccacatccgctctaaccgaaaaggaaggagttagacaacctgaagtctaggtc |
| | | cctatttatttttttatagttatgttagtattaagaacgttatttatatttcaaatttttcttttttttctgtacagacg |
| | | cgtgtacgcatgtaacattatactgaaaaccttgcttgagaaggttttgggacgctcgaaggctttaatttgcaagct |
| Rm3386; | 66 | aggagttagacaacctgaag |
| F test | | |
| oligo | | |
| HA R | 67 | gtaactagtctctcggaatccat |

TABLE 13
Template Nourseothricin Cassette for protease deletion in P. pastoris

| | SEQ | |
|---|---|---|
| | ID | |
| Description | NO: | 5′ to 3′ Sequence |
| Plasmid | 68 | cttcagagtacagaagattaagtgagagaattctaccgttcgtatagcatacattatacgaagttatttcagtaatgtct |
| sequence | | tgtttcttttgttgcagtggtgagccattttgacttcgtgaaagtttctttagaatagttgtttccagaggccaaacatt |
| | | ccacccgtagtaaagtgcaagcgtaggaagaccaagactggcataaatcaggtataagtgtcgagcactggcaggtgatc |
| | | ttctgaaagtttctactagcagataagatccagtagtcatgcatatggcaacaatgtaccgtgtggatctaagaacgcgt |
| | | cctactaaccttcgcattcgttggtccagtttgttgttatcgatcaacgtgacaaggttgtcgattccgcgtaagcatgc |
| | | atacccaaggacgcctgttgcaattccaagtgagccagttccaacaatctttgtaatattagagcacttcattgtgttgc |
| | | gcttgaaagtaaaatgcgaacaaattaagagataatctcgaaaccgcgacttcaaacgccaatatgatgtgcggcacaca |
| | | ataagcgttcatatccgctgggtgactttctcgctttaaaaaattatccgaaaaaatttttgacggctagctcagtccta |
| | | ggtacgctagcattaaagaggagaaaatgactactcttgatgacacagcctacagatataggacatcagttccgggtgac |
| | | gcagaggctatcgaagccttggacggttcattcactactgatacggtgtttagagtcaccgctacaggtgatggcttcac |
| | | cttgagagaggttcctgtagacccacccttaacgaaagttttccctgatgacgaatcggatgacgagtctgatgctggtg |
| | | aggacggtgaccctgattccagaacatttgtcgcatacggagatgatggtgacctggctggctttgttgtggtgtcctac |
| | | agcggatggaatcgtagactcacagttgaggacatcgaagttgcacctgaacatcgtggtcacggtgttggtcgtgcact |
| | | gatgggactggcaacagagtttgctagagaaagaggagccggacatttgtggttagaagtgaccaatgtcaacgctcctg |
| | | ctattcacgcatataggcgaatgggtttcactttgtgcggtcttgatactgctttgtatgacggaactgcttctgatggt |
| | | gaacaagctctttacatgagtatgccatgtccatagcacgtccgacggcggcccacgggtcccaggcctcggagatccgt |
| | | cccccttttcctttgtcgatatcatgtaattagttatgtcacgcttacattcacgccctccccccacatccgctctaacc |
| | | gaaaaggaaggagttagacaacctgaagtctaggtccctatttatttttttatagttatgttagtattaagaacgttatt |
| | | tatatttcaaatttttcttttttttctgtacagacgcgtgtacgcatgtaacattatactgaaaaccttgcttgagaagg |
| | | ttttgggacgctcgaaggctttaatttgcaagctataacttcgtatagcatacattataccttgttatgcggccgcaaga |
| | | agttgattgagactttcaacgag |
| AOX1 pA | 69 | cttcagagtacagaagattaagtgaga |
| terminator | | |
| Lox71 F | 70 | taccgttcgtatagcatacattatacgaagttat |
| pILV5 | 71 | ttcagtaatgtcttgtttcttttgttgcagtggtgagccattttgacttcgtgaaagtttctttagaatagttgtttcca |
| | | gaggccaaacattccacccgtagtaaagtgcaagcgtaggaagaccaagactggcataaatcaggtataagtgtcgagca |
| | | ctggcaggtgatcttctgaaagtttctactagcagataagatccagtagtcatgcatatggcaacaatgtaccgtgtgga |
| | | tctaagaacgcgtcctactaaccttcgcattcgttggtccagtttgttgttatcgatcaacgtgacaaggttgtcgattc |
| | | cgcgtaagcatgcatacccaaggacgcctgttgcaattccaagtgagccagttccaacaatctttgtaatattagagcac |
| | | ttcattgtgttgcgcttgaaagtaaaatgcgaacaaattaagagataatctcgaaaccgcgacttcaaacgccaatatga |
| | | tgtgcggcacacaataagcgttcatatccgctgggtgactttctcgctttaaaaaattatccgaaaaaattt |
| pproRBS | 72 | ttaaagaggagaaa |
| nat | 73 | atgactactcttgatgacacagcctacagatataggacatcagttccgggtgacgcagaggctatcgaagccttggacgg |
| (Nourseo- | | ttcattcactactgatacggtgtttagagtcaccgctacaggtgatggcttcaccttgagagaggttcctgtagacccac |
| thricin | | ccttaacgaaagttttccctgatgacgaatcggatgacgagtctgatgctggtgaggacggtgaccctgattccagaaca |
| resistance) | | tttgtcgcatacggagatgatggtgacctggctggctttgttgtggtgtcctacagcggatggaatcgtagactcacagt |
| | | tgaggacatcgaagttgcacctgaacatcgtggtcacggtgttggtcgtgcactgatgggactggcaacagagtttgcta |
| | | gagaaagaggagccggacatttgtggttagaagtgaccaatgtcaacgctcctgctattcacgcatataggcgaatgggt |
| | | ttcactttgtgcggtcttgatactgctttgtatgacggaactgcttctgatggtgaacaagctctttacatgagtatgcc |
| | | atgtccatag |
| CYC1 | 74 | cacgtccgacggcggcccacgggtcccaggcctcggagatccgtcccccttttcctttgtcgatatcatgtaattagtta |
| terminator | | tgtcacgcttacattcacgccctccccccacatccgctctaaccgaaaaggaaggagttagacaacctgaagtctaggtc |
| | | cctatttatttttttatagttatgttagtattaagaacgttatttatatttcaaatttttcttttttttctgtacagacg |
| | | cgtgtacgcatgtaacattatactgaaaaccttgcttgagaaggttttgggacgctcgaaggctttaatttgcaagct |
| LoxKR3 F | 75 | ataacttcgtatagcatacattataccttgttat |
| HSP82 | 76 | gcggccgcaagaagttgattgagactttcaacgag |

TABLE 14
Exemplary nourseothricin cassettes with HA arms for YPS1-1 and YPS1-2 protease deletion in
P. pastoris

| | SEQ | |
|---|---|---|
| | ID | |
| Description | NO: | 5′ to 3′ Sequence |
| Nourseothricin | 77 | tactacaggctggctgttcctcgcatggtgtttaatgtcctgactgggttttcgtttatcggtattaccggagccaccttg |
| cassette with | | actgtaagggaacgatactggactaagagagtaatgcgaaaggcaacagcgtttctggcgaacctaatcaatgacggttac |
| homology arms | | gagtttactactcctaaagccagtcttattttgctagagcgagtcaacgcttacttaaagggccagggacctaattatgac |
| targeting | | atcgattttgacgagcaggaggcgttcattaaagaaatggaggagttgaggacctctggtggatatgagaacagatactca |
| PAS_chr4_0584 | | tattcaggaaccgatgaaacacccagagatccgggttgcctgtttcttcccattgctttaaataaatggcactttgatgtg |
| (YPS1-1) | | ctagactgcctgaggatatacggtactcaggaagatctggaatctaaattattaagtgttcagcaattggtgttacaatgt |
| | | tgcatgaagcacagtggcatgactccagacatggtctttgcaacggaagtagctcagaagccgaccttcgaagacgacata |
| | | gtttgtgatgatattgacgcttatgcccaggggggtgattgtctagattattgttacacgccaagcaattactccagaact |
| | | ttagaaattcatggcaagattgctaccttacaacgagagctggggctatgctataatattctcggaattttggaccgtttt |
| | | tccgattaaggtttttagctccattgcgccaacccccgctctccagactccttcgttatccagcattcagcatggacaggt |
| | | tcaaaaaataaaatttcttgatatgggtccacttcaaacatgcgcctacctgtaggaaaaaaaaagagaacataaatatgc |
| | | cgcgaacagaaaacgtaatgtactgttctatatataaactgttcagatcaatcataaattctcagtttcaaactttccgct |
| | | cagccagattttattcgtaaagaacgcatcattggctctatgttgaaggatcagttcttgttatgggttgctttgatagcg |
| | | agcgtaccggtttccggcgtgatggcagctcctagcgagtccgggcataacacggttgaaaaacgagatgccaaaaacgtt |
| | | gttggcgttcaacagttggacttcttcagagtacagaagattaagtgagagaattctaccgttcgtatagcatacattata |
| | | cgaagttatttcagtaatgtcttgtttcttttgttgcagtggtgagccattttgacttcgtgaaagtttctttagaatagt |
| | | tgtttccagaggccaaacattccacccgtagtaaagtgcaagcgtaggaagaccaagactggcataaatcaggtataagtg |
| | | tcgagcactggcaggtgatcttctgaaagtttctactagcagataagatccagtagtcatgcatatggcaacaatgtaccg |
| | | tgtggatctaagaacgcgtcctactaaccttcgcattcgttggtccagtttgttgttatcgatcaacgtgacaaggttgtc |
| | | gattccgcgtaagcatgcatacccaaggacgcctgttgcaattccaagtgagccagttccaacaatctttgtaatattaga |
| | | gcacttcattgtgttgcgcttgaaagtaaaatgcgaacaaattaagagataatctcgaaaccgcgacttcaaacgccaata |
| | | tgatgtgcggcacacaataagcgttcatatccgctgggtgactttctcgctttaaaaaattatccgaaaaaatttttgacg |
| | | gctagctcagtcctaggtacgctagcattaaagaggagaaaatgactactcttgatgacacagcctacagatataggacat |
| | | cagttccgggtgacgcagaggctatcgaagccttggacggttcattcactactgatacggtgtttagagtcaccgctacag |
| | | gtgatggcttcaccttgagagaggttcctgtagacccacccttaacgaaagttttccctgatgacgaatcggatgacgagt |
| | | ctgatgctggtgaggacggtgaccctgattccagaacatttgtcgcatacggagatgatggtgacctggctggctttgttg |
| | | tggtgtcctacagcggatggaatcgtagactcacagttgaggacatcgaagttgcacctgaacatcgtggtcacggtgttg |
| | | gtcgtgcactgatgggactggcaacagagtttgctagagaaagaggagccggacatttgtggttagaagtgaccaatgtca |
| | | acgctcctgctattcacgcatataggcgaatgggtttcactttgtgcggtcttgatactgctttgtatgacggaactgctt |
| | | ctgatggtgaacaagctctttacatgagtatgccatgtccatagcacgtccgacggcggcccacgggtcccaggcctcgga |
| | | gatccgtcccccttttcctttgtcgatatcatgtaattagttatgtcacgcttacattcacgccctccccccacatccgct |
| | | ctaaccgaaaaggaaggagttagacaacctgaagtctaggtccctatttatttttttatagttatgttagtattaagaacg |
| | | ttatttatatttcaaatttttcttttttttctgtacagacgcgtgtacgcatgtaacattatactgaaaaccttgcttgag |
| | | aaggttttgggacgctcgaaggctttaatttgcaagctataacttcgtatagcatacattataccttgttatgcggccgca |
| | | agaagttgattgagactttcaacgagggtccccttcagctacctttctctctgtttggtagttattctcggcgtgtgtata |
| | | gtatagtataaaagggcctacattggataggcttcaacattcctcaataaacaaacatccaacatcgcgcattccgcattt |
| | | cgcatttcacatttcgcgcctgccttcctttaggttctttgaatcatcatcaatcgtcgccgtctacatcagagcaggact |
| | | tatctttgccttccccaaaaattgccactccgtcaaatagattcttttgaatccttgactatttttgcctaaataggtttt |
| | | tgttagtttttcttcaaagcccaaaagaaactctatttagattcatccagaaacaatctttttctcaccccatttcgaagt |
| | | gccgtggagcacagacataaaaagatgactaccgttcaacctacagggccagacaggctcaccctgccgcatattctactg |
| | | gaattcaacgatggctcctcgcagcatgcagtgatcgagctaagcatgaacgaggggattaatatatccacccatgagtgg |
| | | aatccatccactaatgagcaatcgccacgggaagagagagcaccaccccaacaatccaatccatcgcatcatccagaatca |
| | | tcgaacatagctactcaaagtcccgctcaggaaaccgagactcagcccggcattccaggactagataggcctgcctttgat |
| | | acctcggcaacggggtcgtcagaacaggttgacccagtacagggaaggatcctggatgatattataggccaatcattaagg |
| | | acttccgaagaagacgataccgaatcccgccagagaccacgagaccagaagaacattatgatcaccgtgaattacttgtac |
| | | gcagacgacacaaattccagaagtgctaatacaaacaaccagacgcccaataacacttctagaacttccgacagtgaacgt |
| | | gtgggctccttatcgttgcacgttccggatctaccagataatgccgacgattactatatcgatgtactcattaaactaacc |
| | | acaagcattgccctcagcgtcatcacgtccatgatcaagaaacgattagggcttagcaggga |
| PAS_chr4_0584 | 78 | tactacaggctggctgttcctcgcatggtgtttaatgtcctgactgggttttcgtttatcggtattaccggagccaccttg |
| Homology | | actgtaagggaacgatactggactaagagagtaatgcgaaaggcaacagcgtttctggcgaacctaatcaatgacggttac |
| Arm 1 | | gagtttactactcctaaagccagtcttattttgctagagcgagtcaacgcttacttaaagggccagggacctaattatgac |
| | | atcgattttgacgagcaggaggcgttcattaaagaaatggaggagttgaggacctctggtggatatgagaacagatactca |
| | | tattcaggaaccgatgaaacacccagagatccgggttgcctgtttcttcccattgctttaaataaatggcactttgatgtg |
| | | ctagactgcctgaggatatacggtactcaggaagatctggaatctaaattattaagtgttcagcaattggtgttacaatgt |
| | | tgcatgaagcacagtggcatgactccagacatggtctttgcaacggaagtagctcagaagccgaccttcgaagacgacata |
| | | gtttgtgatgatattgacgcttatgcccaggggggtgattgtctagattattgttacacgccaagcaattactccagaact |
| | | ttagaaattcatggcaagattgctaccttacaacgagagctggggctatgctataatattctcggaattttggaccgtttt |
| | | tccgattaaggtttttagctccattgcgccaacccccgctctccagactccttcgttatccagcattcagcatggacaggt |
| | | tcaaaaaataaaatttcttgatatgggtccacttcaaacatgcgcctacctgtaggaaaaaaaaagagaacataaatatgc |
| | | cgcgaacagaaaacgtaatgtactgttctatatataaactgttcagatcaatcataaattctcagtttcaaactttccgct |
| | | cagccagattttattcgtaaagaacgcatcattggctctatgttgaaggatcagttcttgttatgggttgctttgatagcg |
| | | agcgtaccggtttccggcgtgatggcagctcctagcgagtccgggcataacacggttgaaaaacgagatgccaaaaacgtt |
| | | gttggcgttcaacagttggactt |
| PAS_chr4_0584 | 79 | ggtccccttcagctacctttctctctgtttggtagttattctcggcgtgtgtatagtatagtataaaagggcctacattgg |
| Homology | | ataggcttcaacattcctcaataaacaaacatccaacatcgcgcattccgcatttcgcatttcacatttcgcgcctgcctt |
| Arm 2 | | cctttaggttctttgaatcatcatcaatcgtcgccgtctacatcagagcaggacttatctttgccttccccaaaaattgcc |
| | | actccgtcaaatagattcttttgaatccttgactatttttgcctaaataggtttttgttagtttttcttcaaagcccaaaa |
| | | gaaactctatttagattcatccagaaacaatctttttctcaccccatttcgaagtgccgtggagcacagacataaaaagat |
| | | gactaccgttcaacctacagggccagacaggctcaccctgccgcatattctactggaattcaacgatggctcctcgcagca |
| | | tgcagtgatcgagctaagcatgaacgaggggattaatatatccacccatgagtggaatccatccactaatgagcaatcgcc |
| | | acgggaagagagagcaccaccccaacaatccaatccatcgcatcatccagaatcatcgaacatagctactcaaagtcccgc |
| | | tcaggaaaccgagactcagcccggcattccaggactagataggcctgcctttgatacctcggcaacggggtcgtcagaaca |
| | | ggttgacccagtacagggaaggatcctggatgatattataggccaatcattaaggacttccgaagaagacgataccgaatc |
| | | ccgccagagaccacgagaccagaagaacattatgatcaccgtgaattacttgtacgcagacgacacaaattccagaagtgc |
| | | taatacaaacaaccagacgcccaataacacttctagaacttccgacagtgaacgtgtgggctccttatcgttgcacgttcc |
| | | ggatctaccagataatgccgacgattactatatcgatgtactcattaaactaaccacaagcattgccctcagcgtcatcac |
| | | gtccatgatcaagaaacgattagggcttagcaggga |
| Nourseothricin | 80 | gccttctcgtgcaatcagagctgttgaaagagagaagagggcacacggaagctgctgttcaattgtgtgaattgaccggat |
| cassette with | | tacaacctgctggagtgataggagagctggttcgtgacgaggacggctctatgatgcgattagacgactgtgttcagtttg |
| homology arms | | gtctccgccacaacgtaaaaattatcaaccttgaccagatcattgaatacatggattccaagaacagctagatacgatgga |
| targeting | | taggaatacagagatatcatgattgaggaacgtaagagctttttcgaaagtgtgagtttgtggtgagggccaggcggtggg |
| PAS_chr3_1157 | | gaggtggtggggagcctccttggtcgaatgtagatatagtaagcaagacacaagagcgcgcgaagtcttcaacgaggcggc |
| (YPS1-2) | | gttgggtcttgtacgcaacgtaatgactacacagttgagcttgtcgcgaaccggtcgacattttgatcatgcatactatgt |
| | | tgagacaccatctcgtactattgcggcaaccagctgtaaatttgactaattaaagctgatgaaggatgcagggcgtcgtca |
| | | attttttgattgattgcatttaattgtttgagccattcaaggctgaatgcccggcaccctagacccttcttgtgagtacta |
| | | taaacccgcaggcagggtacccttggccttctgcgagactaccagtcataacgtatatccacaatgtactagtaatagccc |
| | | cggaaaactctaatcccacagaacgtctaacgcctcctatgtcatcgatacccattcgcactactgccatggcccccctta |
| | | cgtgatcatttcacttactcccgcctaagcttcgcccacatgcctgcgttttgccaagatttactgacgagtttggtttac |
| | | tcatcctctatttataactactagactttcaccattcttcaccaccctcgtgccaatgatcatcaaccacttggtattgac |
| | | agccctcagcattgcactagcaagtgcgcaactccaatcgcctttcacttcagagtacagaagattaagtgagagaattct |
| | | accgttcgtatagcatacattatacgaagttatttcagtaatgtcttgtttcttttgttgcagtggtgagccattttgact |
| | | tcgtgaaagtttctttagaatagttgtttccagaggccaaacattccacccgtagtaaagtgcaagcgtaggaagaccaag |
| | | actggcataaatcaggtataagtgtcgagcactggcaggtgatcttctgaaagtttctactagcagataagatccagtagt |
| | | catgcatatggcaacaatgtaccgtgtggatctaagaacgcgtcctactaaccttcgcattcgttggtccagtttgttgtt |
| | | atcgatcaacgtgacaaggttgtcgattccgcgtaagcatgcatacccaaggacgcctgttgcaattccaagtgagccagt |
| | | tccaacaatctttgtaatattagagcacttcattgtgttgcgcttgaaagtaaaatgcgaacaaattaagagataatctcg |
| | | aaaccgcgacttcaaacgccaatatgatgtgcggcacacaataagcgttcatatccgctgggtgactttctcgctttaaaa |
| | | aattatccgaaaaaatttttgacggctagctcagtcctaggtacgctagcattaaagaggagaaaatgactactcttgatg |
| | | acacagcctacagatataggacatcagttccgggtgacgcagaggctatcgaagccttggacggttcattcactactgata |
| | | cggtgtttagagtcaccgctacaggtgatggcttcaccttgagagaggttcctgtagacccacccttaacgaaagttttcc |
| | | ctgatgacgaatcggatgacgagtctgatgctggtgaggacggtgaccctgattccagaacatttgtcgcatacggagatg |
| | | atggtgacctggctggctttgttgtggtgtcctacagcggatggaatcgtagactcacagttgaggacatcgaagttgcac |
| | | ctgaacatcgtggtcacggtgttggtcgtgcactgatgggactggcaacagagtttgctagagaaagaggagccggacatt |
| | | tgtggttagaagtgaccaatgtcaacgctcctgctattcacgcatataggcgaatgggtttcactttgtgcggtcttgata |
| | | ctgctttgtatgacggaactgcttctgatggtgaacaagctctttacatgagtatgccatgtccatagcacgtccgacggc |
| | | ggcccacgggtcccaggcctcggagatccgtcccccttttcctttgtcgatatcatgtaattagttatgtcacgcttacat |
| | | tcacgccctccccccacatccgctctaaccgaaaaggaaggagttagacaacctgaagtctaggtccctatttattttttt |
| | | atagttatgttagtattaagaacgttatttatatttcaaatttttcttttttttctgtacagacgcgtgtacgcatgtaac |
| | | attatactgaaaaccttgcttgagaaggttttgggacgctcgaaggctttaatttgcaagctataacttcgtatagcatac |
| | | attataccttgttatgcggccgcaagaagttgattgagactttcaacgagctggctctgcttctggtacttcttcaggtgc |
| | | atcttctgctactcaaaatgacgaaacatccactgatcttggagctccagctgcatctttaagtgcaacgccatgtctttt |
| | | tgccatcttgctgctcatgttgtagtagactttttttttcactgagtttttatgtactactgattacattgtgtaggtgta |
| | | atgatgtgcactataatactaatatagtcaaaatgctacagaggaaagtgcaggttgcctgtggtggtttttcttattagc |
| | | accctctgaacactctttacctctaacatcctcagccatgctaatcgcgcataaaataaatcttcgaacttttttccattt |
| | | tatgctcataaagcttccttactgtcaccttatcaaaagagcttttgccactaaagtagtcacacccagaattgctcccga |
| | | atatcgtccaacaatgctaggatctgtggaaagtttgacaaataatttgaacaccttgagcttgaagcttcctgaagttaa |
| | | tatccaaggctcctttccagaaagtaacccagtggaccttttgagaaactacatcactcaagaacttagtaaaatttctgg |
| | | agttgacaaagaattgattttcccagccttggaatggggtaccacactggaaaaaggtgatcttttgatcccagttcctcg |
| | | tctgagaataaagggtgctaatcctaaagatttagccgaacaatgggctgctgcattcccaaagggtggatatcttaaaga |
| | | cgttattgcgcaaggacctttcttgcagttcttttttaacacatcggttctgtacaagttggtgatatctgatgctctgga |
| | | gagaggcgatgactttggtgcacttcctctaggaaagggacaaaaagttatagtggagttttcttctccaaatattgccaa |
| | | acctttccacgctggccatcttagaagtacaatcatcggtggttttatttccaatctgtatgaaaagctgggtcatgaagt |
| | | tatgaggatgaattatttgggagactggggaaaacaatttggtgttcttgcagtaggatttgagcgttacggtgatgaggc |
| | | aaaattaaagactgatccaatcaaccatttgtttgaggtctatgttaaaatcaaccaagatattaaggctcaatcagagtc |
| | | tactgaggagattgcagaagggcaatcattagatgaccaggcaagagcttttttcaagaaaatggaaaatggcgacgaatc |
| | | ggctgtaagcttgtggaaaagattccgtgagttatccattgagaagtacattgatacttatgcccgcctcaacatc |
| PAS_chr3_1157 | 81 | gccttctcgtgcaatcagagctgttgaaagagagaagagggcacacggaagctgctgttcaattgtgtgaattgaccggat |
| Homology | | tacaacctgctggagtgataggagagctggttcgtgacgaggacggctctatgatgcgattagacgactgtgttcagtttg |
| Arm 1 | | gtctccgccacaacgtaaaaattatcaaccttgaccagatcattgaatacatggattccaagaacagctagatacgatgga |
| | | taggaatacagagatatcatgattgaggaacgtaagagctttttcgaaagtgtgagtttgtggtgagggccaggcggtggg |
| | | gaggtggtggggagcctccttggtcgaatgtagatatagtaagcaagacacaagagcgcgcgaagtcttcaacgaggcggc |
| | | gttgggtcttgtacgcaacgtaatgactacacagttgagcttgtcgcgaaccggtcgacattttgatcatgcatactatgt |
| | | tgagacaccatctcgtactattgcggcaaccagctgtaaatttgactaattaaagctgatgaaggatgcagggcgtcgtca |
| | | attttttgattgattgcatttaattgtttgagccattcaaggctgaatgcccggcaccctagacccttcttgtgagtacta |
| | | taaacccgcaggcagggtacccttggccttctgcgagactaccagtcataacgtatatccacaatgtactagtaatagccc |
| | | cggaaaactctaatcccacagaacgtctaacgcctcctatgtcatcgatacccattcgcactactgccatggcccccctta |
| | | cgtgatcatttcacttactcccgcctaagcttcgcccacatgcctgcgttttgccaagatttactgacgagtttggtttac |
| | | tcatcctctatttataactactagactttcaccattcttcaccaccctcgtgccaatgatcatcaaccacttggtattgac |
| | | agccctcagcattgcactagcaagtgcgcaactccaatcgcctttca |
| PAS_chr3_1157 | 82 | ctggctctgcttctggtacttcttcaggtgcatcttctgctactcaaaatgacgaaacatccactgatcttggagctccag |
| Homology | | ctgcatctttaagtgcaacgccatgtctttttgccatcttgctgctcatgttgtagtagactttttttttcactgagtttt |
| Arm 2 | | tatgtactactgattacattgtgtaggtgtaatgatgtgcactataatactaatatagtcaaaatgctacagaggaaagtg |
| | | caggttgcctgtggtggtttttcttattagcaccctctgaacactctttacctctaacatcctcagccatgctaatcgcgc |
| | | ataaaataaatcttcgaacttttttccattttatgctcataaagcttccttactgtcaccttatcaaaagagcttttgcca |
| | | ctaaagtagtcacacccagaattgctcccgaatatcgtccaacaatgctaggatctgtggaaagtttgacaaataatttga |
| | | acaccttgagcttgaagcttcctgaagttaatatccaaggctcctttccagaaagtaacccagtggaccttttgagaaact |
| | | acatcactcaagaacttagtaaaatttctggagttgacaaagaattgattttcccagccttggaatggggtaccacactgg |
| | | aaaaaggtgatcttttgatcccagttcctcgtctgagaataaagggtgctaatcctaaagatttagccgaacaatgggctg |
| | | ctgcattcccaaagggtggatatcttaaagacgttattgcgcaaggacctttcttgcagttcttttttaacacatcggttc |
| | | tgtacaagttggtgatatctgatgctctggagagaggcgatgactttggtgcacttcctctaggaaagggacaaaaagtta |
| | | tagtggagttttcttctccaaatattgccaaacctttccacgctggccatcttagaagtacaatcatcggtggttttattt |
| | | ccaatctgtatgaaaagctgggtcatgaagttatgaggatgaattatttgggagactggggaaaacaatttggtgttcttg |
| | | cagtaggatttgagcgttacggtgatgaggcaaaattaaagactgatccaatcaaccatttgtttgaggtctatgttaaaa |
| | | tcaaccaagatattaaggctcaatcagagtctactgaggagattgcagaagggcaatcattagatgaccaggcaagagctt |
| | | ttttcaagaaaatggaaaatggcgacgaatcggctgtaagcttgtggaaaagattccgtgagttatccattgagaagtaca |
| | | ttgatacttatgcccgcctcaacatc |

TABLE 15
Information on genes and sequences disclosed herein

| S. cerevisiae | S. cerevisiae | P. pastoris | P. pastoris | Open Reading | |
|---|---|---|---|---|---|
| ortholog | ortholog | (K. phaffii) | (K. phaffii) ORF | Frame (ORF) | Peptide |
| standard name | systematic name | gene ID | coordinates (strand) | SEQ ID NO: | SEQ ID NO: |
| SEC72 | YLR292C | PAS_chr2-1_0448 | chr2: 827202- | 1 | 2 |
| | | | 827789(−) | | |
| SBH2 | YER019C-A | PAS_chr2-2_0210 | chr2: 1994927- | 7 | 8 |
| | | | 1995252(+) | | |
| SSS1 | YDR086C | PAS_chr1-1_0023 | chr1: 580942- | 5 | 6 |
| | | | 581136(+) | | |
| SSH1 | YBR283C | PAS_chr1-4_0629 | chr1: 2584924- | 3 | 4 |
| | | | 2586363(−) | | |
| PEP4 | YPL154C | n/a | n/a | 83 | 84 |
| PRC1, mutant | YMR297W | n/a | n/a | 85 | 86 |
| DAP2 | YHR028C | n/a | n/a | 87 | 88 |
| MF(alpha)1, variant | YPL187W | n/a | n/a | 89 | 90 |

TABLE 16
Information on promoters and terminators
for the SSH1 complex genes

| | | | P. pastoris | |
|---|---|---|---|---|
| S. cerevisiae | S. cerevisiae | | (K. phaffii) | |
| ortholog | ortholog | | ORF | Nucleotide |
| standard | systematic | Promoter/ | coordinates | Sequence |
| name | name | Terminator | (strand) | SEQ ID NO: |
| SBH2 | YER019C-A | Promoter | chr2: 1994039- | 97 |
| | | | 1994926(+) | |
| SBH2 | YER019C-A | Terminator | chr2: 1995253- | 100 |
| | | | 1995712(+) | |
| SSS1 | YDR086C | Promoter | chr1: 579989- | 98 |
| | | | 580941(+) | |
| SSS1 | YDR086C | Terminator | chr1: 581137- | 101 |
| | | | 581628(+) | |
| SSH1 | YBR283C | Promoter | chr1: 2586364- | 99 |
| | | | 2587353(−) | |
| SSH1 | YBR283C | Terminator | chr1: 2584553- | 102 |
| | | | 2584923(−) | |

1.-66. (canceled)

67. A Pichia pastoris microorganism, in which the activity of a SEC72 protein has been eliminated or in which a sec72 gene has been deleted, and wherein said microorganism expresses a recombinant silk-like protein comprising a secretion signal peptide derived from yeast.

68. The microorganism of claim 67, wherein said SEC72 protein comprises a polypeptide sequence at least 95% identical to SEQ ID NO: 2.

69. The microorganism of claim 67, further comprising a recombinantly expressed SSH1 translocon complex.

70. The microorganism of claim 69, wherein said SSH1 translocon complex comprises a first polypeptide sequence at least 95% identical to SEQ ID NO: 4, a second polypeptide sequence at least 95% identical to SEQ ID NO: 6, and a third polypeptide sequence at least 95% identical to SEQ ID NO: 8.

71. The microorganism of claim 69, wherein said SSH1 translocon complex comprises a first polypeptide comprising SEQ ID NO: 4, a second polypeptide comprising SEQ ID NO: 6, and a third polypeptide comprising SEQ ID NO: 8.

72. The microorganism of claim 67, further comprising a recombinantly expressed translocon complex.

73. The microorganism of claim 72, wherein said translocon complex is expressed from a recombinant SSH1 gene, a recombinant SSS1 gene, and a recombinant SBH2 gene.

74. The microorganism of claim 73, wherein said SSH1 gene comprises SEQ ID NO: 3.

75. The microorganism of claim 73, wherein said SSH1 gene comprises a polynucleotide sequence at least 95% identical to SEQ ID NO: 3, wherein said SSS1 gene comprises a polynucleotide sequence at least 95% identical to SEQ ID NO: 5, or wherein said SBH2 gene comprises a polynucleotide sequence at least 95% identical to SEQ ID NO: 7.

76. The microorganism of claim 72, wherein said translocon complex comprises an SSH1 protein comprising a polypeptide sequence at least 95% identical to SEQ ID NO: 4, an SSS1 protein comprising a polypeptide sequence at least 95% identical to SEQ ID NO: 6, and an SBH2 protein comprises a polypeptide sequence at least 95% identical to SEQ ID NO: 8.

77. The microorganism of claim 67, wherein activity of a YPS1-1 protease and a YPS1-2 protease have been attenuated or eliminated.

78. The microorganism of claim 77, wherein said YPS1-1 protease comprises a polypeptide sequence at least 95% identical to SEQ ID NO: 10 or wherein said YPS1-2 protease comprises a polypeptide sequence at least 95% identical to SEQ ID NO: 12.

79. The microorganism of claim 67, wherein said secretion signal peptide is selected from the group consisting of: a PEP4 signal sequence, a CPY+4 signal sequence, a DAP2 signal sequence, and a MFa1 signal sequence.

80. The microorganism of claim 67, wherein said secretion signal peptide is selected from the group consisting of: SEQ ID NO: 84, SEQ ID NO: 86, SEQ ID NO: 88, and SEQ ID NO: 90.

81. A method of producing a recombinant protein, comprising:
culturing the microorganism of claim 67 in a culture medium under conditions suitable for expression of the recombinantly expressed protein; and
isolating the recombinant protein from the microorganism or the culture medium.


SEQUENCE LISTING (107 sequences)

SEQ ID NO: 1 - DNA, 588 bp, Pichia pastoris
  source 1..588: mol_type = other DNA
atgctaccattttcgtacgacgtgagctcaaagaaactgaaagtcacaggtgactacgcagacttagaatatgacatacagcagctgaacaccttgagcggagagatcctggccaataaagcagatgttccttctccaccaagtaaggagtcgtttgacaagaaattgtcccacatggctcagaaattacacgagtcggctgtatccaacataaagacaggcaagtatcctgaggctatcaaattgttgacgacgggtcttgaaatggttaacagaaggcccaagtacgagagttttcagatgacgttgagtgaaatgacgatctttattgtcactagagctgacgcttacatgatgaatggagactttgaaggggcattcaatgatgcagatttactggtaacgctcctgccatccattccagataattacattagaagaggggtagcccttttcaagatggggagatacgttgatgcaaaaaacaattttgagagaggactttcatttgacccagataatgcaaaattgaagaaggagttagattttgtgctgaagaagatcgacgaggagaatggagagttatag

SEQ ID NO: 2 - protein, 195 aa, Pichia pastoris
MLPFSYDVSSKKLKVTGDYADLEYDIQQLNTLSGEILANKADVPSPPSKESFDKKLSHMAQKLHESAVSNIKTGKYPEAIKLLTTGLEMVNRRPKYESFQMTLSEMTIFIVTRADAYMMNGDFEGAFNDADLLVTLLPSIPDNYIRRGVALFKMGRYVDAKNNFERGLSFDPDNAKLKKELDFVLKKIDEENGEL

SEQ ID NO: 3 - DNA, 1440 bp, Pichia pastoris
  source 1..1440: mol_type = other DNA
atggcggggttgcgttttttagacattgcaagaccatttgtcagctggatcccggaagttgaacttccttatgaaaactgggggttcgatgaaaagctgatttactcatttttcactgctgccatctatttgattctgtccctgcctatatacggtgtcaaatcctctgaagtcgtggacccagttccccatttgcgttctgccttagggagtgagaagggaacattgctggagcttgggttactgcctgtgattacttcggcatttatcttgcagttgttggctggttggaaagttttcaaagtaaactttgatctggttagtgacagaatattgttccaaactttgcaaaagatcacttcagtcgttatcagcatcgtatatgctgttcttctcacattttgtgactactttactccaggtgtgtccactgataacgtcttgtggtcccaatttctgatcatcttacagatagtggtggtcaacttcttggttactctactcgttgaagtcattgacaaggattacggattttcttcaggagctctattgttgcttgcggtttattccgccaccaacttcgtttttggcacgattggtcttagcaccgtcaacacctccagatcgaacgaatctattggtgctctgattcaattattccgcaatttgagctctaaaccaattggtgttgccatatatgactccttcttcagagtaaaccttcctaacttgactcaattttatctggggattgccattatttgtgtttgtctgttcttgaataatgcaagatacgaagtaccaattaagccaaacaaggttcgtgccatggcctcagcttacccaatcaagctacttttcaatggttctttgccacttctgtacacgtggactgtgctgtacaacttgaaccttattggtttctttgtcttcaagcttaccaacttttctcttttagggaacttcaaagtggacccattcggcaacaactactacgaaattacatctggactgctgtatttattgactcctactttcaacgctgaagctggacttttacccaatgttgctaagccatttgttttcattgccttctatgttggtgttagcactttctttgctagatcgtggtccaacattaacgggtcgtcaggcaaggacattgccaagtttttcaaggctcaaggaatctcattgttaggaaaaagagatgcctctgtgtctaaagagtttaacaccctagttcctgttgcttctgcctctggagctttcctattgtcttttccagttgccgtcgctgagttattgggtggctctggtgttccaacctctatcggaatcggtcttttgagtggtttggctattttggaaactgttttgcaagaatggcaacagtctggaggtgcctcacagttctcccaatacttccagacttcttag

SEQ ID NO: 4 - protein, 479 aa, Pichia pastoris
MAGLRFLDIARPFVSWIPEVELPYENWGFDEKLIYSFFTAAIYLILSLPIYGVKSSEVVDPVPHLRSALGSEKGTLLELGLLPVITSAFILQLLAGWKVFKVNFDLVSDRILFQTLQKITSVVISIVYAVLLTFCDYFTPGVSTDNVLWSQFLIILQIVVVNFLVTLLVEVIDKDYGFSSGALLLLAVYSATNFVFGTIGLSTVNTSRSNESIGALIQLFRNLSSKPIGVAIYDSFFRVNLPNLTQFYLGIAIICVCLFLNNARYEVPIKPNKVRAMASAYPIKLLFNGSLPLLYTWTVLYNLNLIGFFVFKLTNFSLLGNFKVDPFGNNYYEITSGLLYLLTPTFNAEAGLLPNVAKPFVFIAFYVGVSTFFARSWSNINGSSGKDIAKFFKAQGISLLGKRDASVSKEFNTLVPVASASGAFLLSFPVAVAELLGGSGVPTSIGIGLLSGLAILETVLQEWQQSGGASQFSQYFQTS

SEQ ID NO: 5 - DNA, 195 bp, Pichia pastoris
  source 1..195: mol_type = other DNA
atgtcccaaaaagtcaccgacgtccctctggaatttgttaaggaaggttccaaattcatctctaaatgtactaaaccctctcagaaggagtacttaaagatagtaagagctgttggagttgggtttttaatgatgggcgtggttggttacgttgtcaagctcattcatattccaatcagatatttgattgtttaa

SEQ ID NO: 6 - protein, 64 aa, Pichia pastoris
MSQKVTDVPLEFVKEGSKFISKCTKPSQKEYLKIVRAVGVGFLMMGVVGYVVKLIHIPIRYLIV

SEQ ID NO: 7 - DNA, 326 bp, Pichia pastoris
  source 1..326: mol_type = other DNA
atggtaagtgtccagtttgatgagtgcagaatggttccaagttttagaccagttactaatatttaaagtctacagcaattccaggaggacagagaacgttagctaaaagaagagcagcaaacttggataagaaacaggatgaaccaacctccgccagatctgccggtgctggaggttcttcgtctaccatgctaaagttgtacacagacgaggcccaaggtttgaaagttgatcctttaattgttcttgttcttgctgttggtttcattttcagtgtcattggtttgcacgttgttgctaagctgacaggaaagttgatcaactaa

SEQ ID NO: 8 - protein, 86 aa, Pichia pastoris
MSTAIPGGQRTLAKRRAANLDKKQDEPTSARSAGAGGSSSTMLKLYTDEAQGLKVDPLIVLVLAVGFIFSVIGLHVVAKLTGKLIN

SEQ ID NO: 9 - DNA, 1800 bp, Pichia pastoris
  source 1..1800: mol_type = other DNA
atgttgaaggatcagttcttgttatgggttgctttgatagcgagcgtaccggtttccggcgtgatggcagctcctagcgagtccgggcataacacggttgaaaaacgagatgccaaaaacgttgttggcgttcaacagttggacttcagcgttctgaggggtgattccttcgaaagtgcctcttcagagaacgtgcctcggcttgtgaggagagatgacacgctagaagctgagctaatcaaccagcaatcattctacttgtcacgactgaaagttggatcacatcaagcggatattggaatcctagtggacacaggatcctctgatttatgggtaatggactcggtaaacccatactgcagtagccgttcccgcgtgaagagagatatacacgatgagaagatcgccgaatgggatcccatcaatctcaagaaaaatgaaacttctcagaataaaaatttttgggattggctcgttggaactagcactagttctccttccaccgccacggcaactggtagtggtagtggtagtggtagtggtagtggtagtggtagtgctgccacagccgtatcggtaagttctgcacaggcaacattggattgctctacgtatggaacgtttgatcacgctgattcctcgacgttccatgacaataatacagactttttcatctcatacgctgataccacttttgcttcaggaatctggggttatgacgacgtcattatcgacggcatagaggtgaaagaactttccttcgccgttgcagacatgaccaattcctctattggtgtgttaggtattggactgaaaggcctagaatccacatatgctagtgcatcttcggtcagtgaaatgtatcagtatgacaatttgccagccaagatggtcaccgatgggttgatcaacaaaaatgcatactccttgtacttgaactccaaggacgcctcaagtggttccatcctctttggaggtgtggatcatgaaaaatattcgggacaattgttgacagttccagtcatcaacacactcgcttccagtggttacagagaggcaattcgtttacaaattactttaaatggaatagatgtgaaaaagggttctgaccagggaactcttttacaagggagatttgctgcattattggactctggagctacgctaacgtatgctccttcttctgttttaaattcaattggccggaacctgggcggctcctatgattcgtcaagacaagcttataccattcgttgtgtttctgcatcagataccacttctctggtattcaattttgggggtgctacagtggaagtttccctgtacgatctacagattgcaacatattacaccgggggaagtgccacgcaatgtcttattggaatattcagctctggaagtgatgagtttgtgctcggtgataccttcttgaggtcagcctacgtggtttacgatcttgatgggcttgaagtgtcgcttgcccaagccaacttcaacgaaaccgattctgatgttgaggctattacctccagtgtaccttccgctactcgtgcatccggatacagttctacatggtctggttctgccagcggtacagtttacacttcggttcagatggaatccggtgctgcttccagctccaactcttctggatcgaatatgggttcctcttcctcatcgtcctcttcatcgtcctcgacttccagtggagacgaagaaggagggagctccgccaacagggtccccttcagctacctttctctctgtttggtagttattctcggcgtgtgtatagtatag

SEQ ID NO: 10 - protein, 599 aa, Pichia pastoris
MLKDQFLLWVALIASVPVSGVMAAPSESGHNTVEKRDAKNVVGVQQLDFSVLRGDSFESASSENVPRLVRRDDTLEAELINQQSFYLSRLKVGSHQADIGILVDTGSSDLWVMDSVNPYCSSRSRVKRDIHDEKIAEWDPINLKKNETSQNKNFWDWLVGTSTSSPSTATATGSGSGSGSGSGSGSAATAVSVSSAQATLDCSTYGTFDHADSSTFHDNNTDFFISYADTTFASGIWGYDDVIIDGIEVKELSFAVADMTNSSIGVLGIGLKGLESTYASASSVSEMYQYDNLPAKMVTDGLINKNAYSLYLNSKDASSGSILFGGVDHEKYSGQLLTVPVINTLASSGYREAIRLQITLNGIDVKKGSDQGTLLQGRFAALLDSGATLTYAPSSVLNSIGRNLGGSYDSSRQAYTIRCVSASDTTSLVFNFGGATVEVSLYDLQIATYYTGGSATQCLIGIFSSGSDEFVLGDTFLRSAYVVYDLDGLEVSLAQANFNETDSDVEAITSSVPSATRASGYSSTWSGSASGTVYTSVQMESGAASSSNSSGSNMGSSSSSSSSSSSTSSGDEEGGSSANRVPFSYLSLCLVVILGVCIV

SEQ ID NO: 11 - DNA, 1781 bp, Pichia pastoris
  source 1..1781: mol_type = other DNA
atgatcatcaaccacttggtattgacagccctcagcattgcactagcaagtgcgcaactccaatcgcctttcaaggctaacaagttgccattcaaaaagtttatcattccaacgacccaaaggaccgtttaattaagagagatgactacgagtccctcgacttgagacacatcggagtcttgtacactgcagagatccaaattggatctgacgaaactgaaattgaggtcattgtcgacactggttctgccgacttgtgggtcatcgattccgacgctgccgtctgtgagttatcctacgatgagattgaggccaatagcttttcctcggcttctgccaaattcatggacaagatagctcctccatcacaagagctcctggatgggctgagtgagtttggatttgctctcgatggtgaaatttctcaatacctagccgataaatctggacgtgtttcgaaaagagaggaaaatcaacaagatttcaacattaaccgtgacgagcctgtgtgtgaacagtttggttccttcgattctagttcttccgacactttccaaagcaacaattcagcttttggtattgcttaccttgatggaaccactgctaacggaacttgggtcagggacacagtccgcatcggcgactttgccatcagccaacagagttttgccttagtcaacatcacagataactacatgggaatcttgggtctcggtcctgctacccaacaaaccaccaatagtaacccaattgcagcaaacagatttacttatgatggtgttgtggattcattgcggtcccaaggatttatcaattcagcatcgttttctgtttacttgtctccagatgaagataacgagcacgacgaattcagcgacggagaaattttatttggtgctattgatagggccaagatagacgggccatttagacttttcccatatgtcaatccttacaaaccagtttaccccgatcaatatacttcctacgttacagtgtccacaattgcggtgtcttcgtcagatgaaactctcattattgaaagacgtcctcgtttggcattaatcgatacaggtgccaccttctcctatttgccaacctacccattgattcgtttagcgttttccatccatggaggctttgaatatgtttctcaattgggactatttgtcattcgtacaagttctctgtctgttgctagaaataaggtgattgagttcaagtttggtgaagacgttgtgatccaatccccagtttctgatcatctattggacgtctcaggcctttttactgatggccaacaatactccgcattaactgtacgtgaaagtcttgacggactttccattctaggtgatacattcatcaaatcggcctacttattctttgacaatgaaaacagccagctgggtattggtcagatcaacgtcactgatgacgaggatattgaggtggtcggtgatttcactattgaacgagacccagcctactcctctacttggtctagcgatttacctcatgaaacacccactagggctttgagtactgcttcagggggaggccttggtaccggaataaacacggccacaagtcgtgcaagttctcgttccacatctggctctacttcacgaacttcttctacatctggctctgcttctggtacttcttcaggtgcatcttctgctactcaaaatgacgaaacatccactgatcttggagctccagctgcatctttaagtgcaacgccatgtctttttgccatcttgctgctcatgttgtag

SEQ ID NO: 12 - protein, 562 aa, Pichia pastoris
MIINHLVLTALSIALANDYESLDLRHIGVLYTAEIQIGSDETEIEVIVDTGSADLWVIDSDAAVCELSYDEIEANSFSSASAKFMDKIAPPSQELLDGLSEFGFALDGEISQYLADKSGRVSKREENQQDFNINRDEPVCEQFGSFDSSSSDTFQSNNSAFGIAYLDGTTANGTWVRDTVRIGDFAISQQSFALVNITDNYMGILGLGPATQQTTNSNPIAANRFTYDGVVDSLRSQGFINSASFSVYLSPDEDNEHDEFSDGEILFGAIDRAKIDGPFRLFPYVNPYKPVYPDQYTSYVTVSTIAVSSSDETLIIERRPRLALIDTGATFSYLPTYPLIRLAFSIHGGFEYVSQLGLFVIRTSSLSVARNKVIEFKFGEDVVIQSPVSDHLLDVSGLFTDGQQYSALTVRESLDGLSILGDTFIKSAYLFFDNENSQLGIGQINVTDDEDIEVVGDFTIERDPAYSSTWSSDLPHETPTRALSTASGGGLGTGINTATSRASSRSTSGSTSRTSSTSGSASGTSSGASSATQNDETSTDLGAPAASLSATPCLFAILLLML

SEQ ID NO: 13 - protein, 1800 aa, synthetic construct
  VARIANT 1..1800: note = Description of Artificial Sequence: Synthetic polypeptide
  VARIANT 7..11: note = MISC_FEATURE - This region may encompass "SGGQQ," "GAGQQ," "GQGPY," "AGQQ" or "SQ," wherein some positions may be absent
  VARIANT 15..19: note = MISC_FEATURE - This region may encompass "SGGQQ," "GAGQQ," "GQGPY," "AGQQ" or "SQ," wherein some positions may be absent
  VARIANT 23..27: note = MISC_FEATURE - This region may encompass "SGGQQ," "GAGQQ," "GQGPY," "AGQQ" or "SQ," wherein some positions may be absent
  VARIANT 31..35: note = MISC_FEATURE - This region may encompass "SGGQQ," "GAGQQ," "GQGPY," "AGQQ" or "SQ," wherein some positions may be absent
  VARIANT 39..43: note = MISC_FEATURE - This region may encompass "SGGQQ," "GAGQQ," "GQGPY," "AGQQ" or "SQ," wherein some positions may be absent
  VARIANT 47..51: note = MISC_FEATURE - This region may encompass "SGGQQ," "GAGQQ," "GQGPY," "AGQQ" or "SQ," wherein some positions may be absent
  VARIANT 55..59: note = MISC_FEATURE - This region may encompass "SGGQQ," "GAGQQ," "GQGPY," "AGQQ" or "SQ," wherein some positions may be absent
  VARIANT 63..67: note = MISC_FEATURE - This region may encompass "SGGQQ," "GAGQQ," "GQGPY," "AGQQ" or "SQ," wherein some positions may be absent
  VARIANT 4..67: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 71..90: note = MISC_FEATURE - This region may encompass 6-20 residues
  VARIANT 97..101: note = MISC_FEATURE - This region may encompass "SGGQQ," "GAGQQ," "GQGPY," "AGQQ" or "SQ," wherein some positions may be absent
  VARIANT 105..109: note = MISC_FEATURE - This region may encompass "SGGQQ," "GAGQQ," "GQGPY," "AGQQ" or "SQ," wherein some positions may be absent
  VARIANT 113..117: note = MISC_FEATURE - This region may encompass "SGGQQ," "GAGQQ," "GQGPY," "AGQQ" or "SQ," wherein some positions may be absent
  VARIANT 121..125: note = MISC_FEATURE - This region may encompass "SGGQQ," "GAGQQ," "GQGPY," "AGQQ" or "SQ," wherein some positions may be absent
  VARIANT 129..133: note = MISC_FEATURE - This region may encompass "SGGQQ," "GAGQQ," "GQGPY," "AGQQ" or "SQ," wherein some positions may be absent
  VARIANT 137..141: note = MISC_FEATURE - This region may encompass "SGGQQ," "GAGQQ," "GQGPY," "AGQQ" or "SQ," wherein some positions may be absent
  VARIANT 145..149: note = MISC_FEATURE - This region may encompass "SGGQQ," "GAGQQ," "GQGPY," "AGQQ" or "SQ," wherein some positions may be absent
  VARIANT 153..157: note = MISC_FEATURE - This region may encompass "SGGQQ," "GAGQQ," "GQGPY," "AGQQ" or "SQ," wherein some positions may be absent
  VARIANT 94..157: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 161..180: note = MISC_FEATURE - This region may encompass 6-20 residues
  VARIANT 187..191: note = MISC_FEATURE - This region may encompass "SGGQQ," "GAGQQ," "GQGPY," "AGQQ" or "SQ," wherein some positions may be absent
  VARIANT 195..199: note = MISC_FEATURE - This region may encompass "SGGQQ," "GAGQQ," "GQGPY," "AGQQ" or "SQ," wherein some positions may be absent
  VARIANT 203..207: note = MISC_FEATURE - This region may encompass "SGGQQ," "GAGQQ," "GQGPY," "AGQQ" or "SQ," wherein some positions may be absent
  VARIANT 211..215: note = MISC_FEATURE - This region may encompass "SGGQQ," "GAGQQ," "GQGPY," "AGQQ" or "SQ," wherein some positions may be absent
  VARIANT 219..223: note = MISC_FEATURE - This region may encompass "SGGQQ," "GAGQQ," "GQGPY," "AGQQ" or "SQ," wherein some positions may be absent
  VARIANT 227..231: note = MISC_FEATURE - This region may encompass "SGGQQ," "GAGQQ," "GQGPY," "AGQQ" or "SQ," wherein some positions may be absent
  VARIANT 235..239: note = MISC_FEATURE - This region may encompass "SGGQQ," "GAGQQ," "GQGPY," "AGQQ" or "SQ," wherein some positions may be absent
  VARIANT 243..247: note = MISC_FEATURE - This region may encompass "SGGQQ," "GAGQQ," "GQGPY," "AGQQ" or "SQ," wherein some positions may be absent
  VARIANT 184..247: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 251..270: note = MISC_FEATURE - This region may encompass 6-20 residues
  VARIANT 277..281: note = MISC_FEATURE - This region may encompass "SGGQQ," "GAGQQ," "GQGPY," "AGQQ" or "SQ," wherein some positions may be absent
  VARIANT 285..289: note = MISC_FEATURE - This region may encompass "SGGQQ," "GAGQQ," "GQGPY," "AGQQ" or "SQ," wherein some positions may be absent
  VARIANT 293..297: note = MISC_FEATURE - This region may encompass "SGGQQ," "GAGQQ," "GQGPY," "AGQQ" or "SQ," wherein some positions may be absent
  VARIANT 301..305: note = MISC_FEATURE - This region may encompass "SGGQQ," "GAGQQ," "GQGPY," "AGQQ" or "SQ," wherein some positions may be absent
  VARIANT 309..313: note = MISC_FEATURE - This region may encompass "SGGQQ," "GAGQQ," "GQGPY," "AGQQ" or "SQ," wherein some positions may be absent
  VARIANT 317..321: note = MISC_FEATURE - This region may encompass "SGGQQ," "GAGQQ," "GQGPY," "AGQQ" or "SQ," wherein some positions may be absent
  VARIANT 325..329: note = MISC_FEATURE - This region may encompass "SGGQQ," "GAGQQ," "GQGPY," "AGQQ" or "SQ," wherein some positions may be absent
  VARIANT 333..337: note = MISC_FEATURE - This region may encompass "SGGQQ," "GAGQQ," "GQGPY," "AGQQ" or "SQ," wherein some positions may be absent
  VARIANT 274..337: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 341..360: note = MISC_FEATURE - This region may encompass 6-20 residues
  VARIANT 367..371: note = MISC_FEATURE - This region may encompass "SGGQQ," "GAGQQ," "GQGPY," "AGQQ" or "SQ," wherein some positions may be absent
  VARIANT 375..379: note = MISC_FEATURE - This region may encompass "SGGQQ," "GAGQQ," "GQGPY," "AGQQ" or "SQ," wherein some positions may be absent
  VARIANT 383..387: note = MISC_FEATURE - This region may encompass "SGGQQ," "GAGQQ," "GQGPY," "AGQQ" or "SQ," wherein some positions may be absent
  VARIANT 391..395: note = MISC_FEATURE - This region may encompass "SGGQQ," "GAGQQ," "GQGPY," "AGQQ" or "SQ," wherein some positions may be absent
  VARIANT 399..403: note = MISC_FEATURE - This region may encompass "SGGQQ," "GAGQQ," "GQGPY," "AGQQ" or "SQ," wherein some positions may be absent
  VARIANT 407..411: note = MISC_FEATURE - This region may encompass "SGGQQ," "GAGQQ," "GQGPY," "AGQQ" or "SQ," wherein some positions may be absent
  VARIANT 415..419: note = MISC_FEATURE - This region may encompass "SGGQQ," "GAGQQ," "GQGPY," "AGQQ" or "SQ," wherein some positions may be absent
  VARIANT 423..427: note = MISC_FEATURE - This region may encompass "SGGQQ," "GAGQQ," "GQGPY," "AGQQ" or "SQ," wherein some positions may be absent
  VARIANT 364..427: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 431..450: note = MISC_FEATURE - This region may encompass 6-20 residues
  VARIANT 457..461: note = MISC_FEATURE - This region may encompass "SGGQQ," "GAGQQ," "GQGPY," "AGQQ" or "SQ," wherein some positions may be absent
  VARIANT 465..469: note = MISC_FEATURE - This region may encompass "SGGQQ," "GAGQQ," "GQGPY," "AGQQ" or "SQ," wherein some positions may be absent
  VARIANT 473..477: note = MISC_FEATURE - This region may encompass "SGGQQ," "GAGQQ," "GQGPY," "AGQQ" or "SQ," wherein some positions may be absent
  VARIANT 481..485: note = MISC_FEATURE - This region may encompass "SGGQQ," "GAGQQ," "GQGPY," "AGQQ" or "SQ," wherein some positions may be absent
  VARIANT 489..493: note = MISC_FEATURE - This region may encompass "SGGQQ," "GAGQQ," "GQGPY," "AGQQ" or "SQ," wherein some positions may be absent
  VARIANT 497..501: note = MISC_FEATURE - This region may encompass "SGGQQ," "GAGQQ," "GQGPY," "AGQQ" or "SQ," wherein some positions may be absent
  VARIANT 505..509: note = MISC_FEATURE - This region may encompass "SGGQQ," "GAGQQ," "GQGPY," "AGQQ" or "SQ," wherein some positions may be absent
  VARIANT 513..517: note = MISC_FEATURE - This region may encompass "SGGQQ," "GAGQQ," "GQGPY," "AGQQ" or "SQ," wherein some positions may be absent
  VARIANT 454..517: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 521..540: note = MISC_FEATURE - This region may encompass 6-20 residues
  VARIANT 547..551: note = MISC_FEATURE - This region may encompass "SGGQQ," "GAGQQ," "GQGPY," "AGQQ" or "SQ," wherein some positions may be absent
  VARIANT 555..559: note = MISC_FEATURE - This region may encompass "SGGQQ," "GAGQQ," "GQGPY," "AGQQ" or "SQ," wherein some positions may be absent
  VARIANT 563..567: note = MISC_FEATURE - This region may encompass "SGGQQ," "GAGQQ," "GQGPY," "AGQQ" or "SQ," wherein some positions may be absent
  VARIANT 571..575: note = MISC_FEATURE - This region may encompass "SGGQQ," "GAGQQ," "GQGPY," "AGQQ" or "SQ," wherein some positions may be absent
  VARIANT 579..583: note = MISC_FEATURE - This region may encompass "SGGQQ," "GAGQQ," "GQGPY," "AGQQ" or "SQ," wherein some positions may be absent
  VARIANT 587..591: note = MISC_FEATURE - This region may encompass "SGGQQ," "GAGQQ," "GQGPY," "AGQQ" or "SQ," wherein some positions may be absent
  VARIANT 595..599: note = MISC_FEATURE - This region may encompass "SGGQQ," "GAGQQ," "GQGPY," "AGQQ" or "SQ," wherein some positions may be absent
  VARIANT 603..607: note = MISC_FEATURE - This region may encompass "SGGQQ," "GAGQQ," "GQGPY," "AGQQ" or "SQ," wherein some positions may be absent
  VARIANT 544..607: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 611..630: note = MISC_FEATURE - This region may encompass 6-20 residues
  VARIANT 637..641: note = MISC_FEATURE - This region may encompass "SGGQQ," "GAGQQ," "GQGPY," "AGQQ" or "SQ," wherein some positions may be absent
  VARIANT 645..649: note = MISC_FEATURE - This region may encompass "SGGQQ," "GAGQQ," "GQGPY," "AGQQ" or "SQ," wherein some positions may be absent
  VARIANT 653..657: note = MISC_FEATURE - This region may encompass "SGGQQ," "GAGQQ," "GQGPY," "AGQQ" or "SQ," wherein some positions may be absent
  VARIANT 661..665: note = MISC_FEATURE - This region may encompass "SGGQQ," "GAGQQ," "GQGPY," "AGQQ" or "SQ," wherein some positions may be absent
  VARIANT 669..673: note = MISC_FEATURE - This region may encompass "SGGQQ," "GAGQQ," "GQGPY," "AGQQ" or "SQ," wherein some positions may be absent
  VARIANT 677..681: note = MISC_FEATURE - This region may encompass "SGGQQ," "GAGQQ," "GQGPY," "AGQQ" or "SQ," wherein some positions may be absent
  VARIANT 685..689: note = MISC_FEATURE - This region may encompass "SGGQQ," "GAGQQ," "GQGPY," "AGQQ" or "SQ," wherein some positions may be absent
  VARIANT 693..697: note = MISC_FEATURE - This region may encompass "SGGQQ," "GAGQQ," "GQGPY," "AGQQ" or "SQ," wherein some positions may be absent
  VARIANT 634..697: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 701..720: note = MISC_FEATURE - This region may encompass 6-20 residues
  VARIANT 727..731: note = MISC_FEATURE - This region may encompass "SGGQQ," "GAGQQ," "GQGPY," "AGQQ" or "SQ," wherein some positions may be absent
  VARIANT 735..739: note = MISC_FEATURE - This region may encompass "SGGQQ," "GAGQQ," "GQGPY," "AGQQ" or "SQ," wherein some positions may be absent
  VARIANT 743..747: note = MISC_FEATURE - This region may encompass "SGGQQ," "GAGQQ," "GQGPY," "AGQQ" or "SQ," wherein some positions may be absent
  VARIANT 751..755: note = MISC_FEATURE - This region may encompass "SGGQQ," "GAGQQ," "GQGPY," "AGQQ" or "SQ," wherein some positions may be absent
  VARIANT 759..763: note = MISC_FEATURE - This region may encompass "SGGQQ," "GAGQQ," "GQGPY," "AGQQ" or "SQ," wherein some positions may be absent
  VARIANT 767..771: note = MISC_FEATURE - This region may encompass "SGGQQ," "GAGQQ," "GQGPY," "AGQQ" or "SQ," wherein some positions may be absent
  VARIANT 775..779: note = MISC_FEATURE - This region may encompass "SGGQQ," "GAGQQ," "GQGPY," "AGQQ" or "SQ," wherein some positions may be absent
  VARIANT 783..787: note = MISC_FEATURE - This region may encompass "SGGQQ," "GAGQQ," "GQGPY," "AGQQ" or "SQ," wherein some positions may be absent
  VARIANT 724..787: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 791..810: note = MISC_FEATURE - This region may encompass 6-20 residues
  VARIANT 817..821: note = MISC_FEATURE - This region may encompass "SGGQQ," "GAGQQ," "GQGPY," "AGQQ" or "SQ," wherein some positions may be absent
  VARIANT 825..829: note = MISC_FEATURE - This region may encompass "SGGQQ," "GAGQQ," "GQGPY," "AGQQ" or "SQ," wherein some positions may be absent
  VARIANT 833..837: note = MISC_FEATURE - This region may encompass "SGGQQ," "GAGQQ," "GQGPY," "AGQQ" or "SQ," wherein some positions may be absent
  VARIANT 841..845: note = MISC_FEATURE - This region may encompass "SGGQQ," "GAGQQ," "GQGPY," "AGQQ" or "SQ," wherein some positions may be absent
  VARIANT 849..853: note = MISC_FEATURE - This region may encompass "SGGQQ," "GAGQQ," "GQGPY," "AGQQ" or "SQ," wherein some positions may be absent
  VARIANT 857..861: note = MISC_FEATURE - This region may encompass "SGGQQ," "GAGQQ," "GQGPY," "AGQQ" or "SQ," wherein some positions may be absent
  VARIANT 865..869: note = MISC_FEATURE - This region may encompass "SGGQQ," "GAGQQ," "GQGPY," "AGQQ" or "SQ," wherein some positions may be absent
  VARIANT 873..877: note = MISC_FEATURE - This region may encompass "SGGQQ," "GAGQQ," "GQGPY," "AGQQ" or "SQ," wherein some positions may be absent
  VARIANT 814..877: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 881..900: note = MISC_FEATURE - This region may encompass 6-20 residues
  VARIANT 907..911: note = MISC_FEATURE - This region may encompass "SGGQQ," "GAGQQ," "GQGPY," "AGQQ" or "SQ," wherein some positions may be absent
  VARIANT 915..919: note = MISC_FEATURE - This region may encompass "SGGQQ," "GAGQQ," "GQGPY," "AGQQ" or "SQ," wherein some positions may be absent
  VARIANT 923..927: note = MISC_FEATURE - This region may encompass "SGGQQ," "GAGQQ," "GQGPY," "AGQQ" or "SQ," wherein some positions may be absent
  VARIANT 931..935: note = MISC_FEATURE - This region may encompass "SGGQQ," "GAGQQ," "GQGPY," "AGQQ" or "SQ," wherein some positions may be absent
  VARIANT 939..943: note = MISC_FEATURE - This region may encompass "SGGQQ," "GAGQQ," "GQGPY," "AGQQ" or "SQ," wherein some positions may be absent
  VARIANT 947..951: note = MISC_FEATURE - This region may encompass "SGGQQ," "GAGQQ," "GQGPY," "AGQQ" or "SQ," wherein some positions may be absent
  VARIANT 955..959: note = MISC_FEATURE - This region may encompass "SGGQQ," "GAGQQ," "GQGPY," "AGQQ" or "SQ," wherein some positions may be absent
  VARIANT 963..967: note = MISC_FEATURE - This region may encompass "SGGQQ," "GAGQQ," "GQGPY," "AGQQ" or "SQ," wherein some positions may be absent
  VARIANT 904..967: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 971..990: note = MISC_FEATURE - This region may encompass 6-20 residues
  VARIANT 997..1001: note = MISC_FEATURE - This region may encompass "SGGQQ," "GAGQQ," "GQGPY," "AGQQ" or "SQ," wherein some positions may be absent
  VARIANT 1005..1009: note = MISC_FEATURE - This region may encompass "SGGQQ," "GAGQQ," "GQGPY," "AGQQ" or "SQ," wherein some positions may be absent
  VARIANT 1013..1017: note = MISC_FEATURE - This region may encompass "SGGQQ," "GAGQQ," "GQGPY," "AGQQ" or "SQ," wherein some positions may be absent
  VARIANT 1021..1025: note = MISC_FEATURE - This region may encompass "SGGQQ," "GAGQQ," "GQGPY," "AGQQ" or "SQ," wherein some positions may be absent
  VARIANT 1029..1033: note = MISC_FEATURE - This region may encompass "SGGQQ," "GAGQQ," "GQGPY," "AGQQ" or "SQ," wherein some positions may be absent
  VARIANT 1037..1041: note = MISC_FEATURE - This region may encompass "SGGQQ," "GAGQQ," "GQGPY," "AGQQ" or "SQ," wherein some positions may be absent
  VARIANT 1045..1049: note = MISC_FEATURE - This region may encompass "SGGQQ," "GAGQQ," "GQGPY," "AGQQ" or "SQ," wherein some positions may be absent
  VARIANT 1053..1057: note = MISC_FEATURE - This region may encompass "SGGQQ," "GAGQQ," "GQGPY," "AGQQ" or "SQ," wherein some positions may be absent
  VARIANT 994..1057: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 1061..1080: note = MISC_FEATURE - This region may encompass 6-20 residues
  VARIANT 1087..1091: note = MISC_FEATURE - This region may encompass "SGGQQ," "GAGQQ," "GQGPY," "AGQQ" or "SQ," wherein some positions may be absent
  VARIANT 1095..1099: note = MISC_FEATURE - This region may encompass "SGGQQ," "GAGQQ," "GQGPY," "AGQQ" or "SQ," wherein some positions may be absent
  VARIANT 1103..1107: note = MISC_FEATURE - This region may encompass "SGGQQ," "GAGQQ," "GQGPY," "AGQQ" or "SQ," wherein some positions may be absent
  VARIANT 1111..1115: note = MISC_FEATURE - This region may encompass "SGGQQ," "GAGQQ," "GQGPY," "AGQQ" or "SQ," wherein some positions may be absent
  VARIANT 1119..1123: note = MISC_FEATURE - This region may encompass "SGGQQ," "GAGQQ," "GQGPY," "AGQQ" or "SQ," wherein some positions may be absent
  VARIANT 1127..1131: note = MISC_FEATURE - This region may encompass "SGGQQ," "GAGQQ," "GQGPY," "AGQQ" or "SQ," wherein some positions may be absent
  VARIANT 1135..1139: note = MISC_FEATURE - This region may encompass "SGGQQ," "GAGQQ," "GQGPY," "AGQQ" or "SQ," wherein some positions may be absent
  VARIANT 1143..1147: note = MISC_FEATURE - This region may encompass "SGGQQ," "GAGQQ," "GQGPY," "AGQQ" or "SQ," wherein some positions may be absent
  VARIANT 1084..1147: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 1151..1170: note = MISC_FEATURE - This region may encompass 6-20 residues
  VARIANT 1177..1181: note = MISC_FEATURE - This region may encompass "SGGQQ," "GAGQQ," "GQGPY," "AGQQ" or "SQ," wherein some positions may be absent
  VARIANT 1185..1189: note = MISC_FEATURE - This region may encompass "SGGQQ," "GAGQQ," "GQGPY," "AGQQ" or "SQ," wherein some positions may be absent
  VARIANT 1193..1197: note = MISC_FEATURE - This region may encompass "SGGQQ," "GAGQQ," "GQGPY," "AGQQ" or "SQ," wherein some positions may be absent
  VARIANT 1201..1205: note = MISC_FEATURE - This region may encompass "SGGQQ," "GAGQQ," "GQGPY," "AGQQ" or "SQ," wherein some positions may be absent
  VARIANT 1209..1213: note = MISC_FEATURE - This region may encompass "SGGQQ," "GAGQQ," "GQGPY," "AGQQ" or "SQ," wherein some positions may be absent
  VARIANT 1217..1221: note = MISC_FEATURE - This region may encompass "SGGQQ," "GAGQQ," "GQGPY," "AGQQ" or "SQ," wherein some positions may be absent
  VARIANT 1225..1229: note = MISC_FEATURE - This region may encompass "SGGQQ," "GAGQQ," "GQGPY," "AGQQ" or "SQ," wherein some positions may be absent
  VARIANT 1233..1237: note = MISC_FEATURE - This region may encompass "SGGQQ," "GAGQQ," "GQGPY," "AGQQ" or "SQ," wherein some positions may be absent
  VARIANT 1174..1237: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 1241..1260: note = MISC_FEATURE - This region may encompass 6-20 residues
  VARIANT 1267..1271: note = MISC_FEATURE - This region may encompass "SGGQQ," "GAGQQ," "GQGPY," "AGQQ" or "SQ," wherein some positions may be absent
  VARIANT 1275..1279: note = MISC_FEATURE - This region may encompass "SGGQQ," "GAGQQ," "GQGPY," "AGQQ" or "SQ," wherein some positions may be absent
  VARIANT 1283..1287: note = MISC_FEATURE - This region may encompass "SGGQQ," "GAGQQ," "GQGPY," "AGQQ" or "SQ," wherein some positions may be absent
  VARIANT 1291..1295: note = MISC_FEATURE - This region may encompass "SGGQQ," "GAGQQ," "GQGPY," "AGQQ" or "SQ," wherein some positions may be absent
  VARIANT 1299..1303: note = MISC_FEATURE - This region may encompass "SGGQQ," "GAGQQ," "GQGPY," "AGQQ" or "SQ," wherein some positions may be absent
  VARIANT 1307..1311: note = MISC_FEATURE - This region may encompass "SGGQQ," "GAGQQ," "GQGPY," "AGQQ" or "SQ," wherein some positions may be absent
  VARIANT 1315..1319: note = MISC_FEATURE - This region may encompass "SGGQQ," "GAGQQ," "GQGPY," "AGQQ" or "SQ," wherein some positions may be absent
  VARIANT 1323..1327: note = MISC_FEATURE - This region may encompass "SGGQQ," "GAGQQ," "GQGPY," "AGQQ" or "SQ," wherein some positions may be absent
  VARIANT 1264..1327: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 1331..1350: note = MISC_FEATURE - This region may encompass 6-20 residues
  VARIANT 1357..1361: note = MISC_FEATURE - This region may encompass "SGGQQ," "GAGQQ," "GQGPY," "AGQQ" or "SQ," wherein some positions may be absent
  VARIANT 1365..1369: note = MISC_FEATURE - This region may encompass "SGGQQ," "GAGQQ," "GQGPY," "AGQQ" or "SQ," wherein some positions may be absent
  VARIANT 1373..1377: note = MISC_FEATURE - This region may encompass "SGGQQ," "GAGQQ," "GQGPY," "AGQQ" or "SQ," wherein some positions may be absent
  VARIANT 1381..1385: note = MISC_FEATURE - This region may encompass "SGGQQ," "GAGQQ," "GQGPY," "AGQQ" or "SQ," wherein some positions may be absent
  VARIANT 1389..1393: note = MISC_FEATURE - This region may encompass "SGGQQ," "GAGQQ," "GQGPY," "AGQQ" or "SQ," wherein some positions may be absent
  VARIANT 1397..1401: note = MISC_FEATURE - This region may encompass "SGGQQ," "GAGQQ," "GQGPY," "AGQQ" or "SQ," wherein some positions may be absent
  VARIANT 1405..1409: note = MISC_FEATURE - This region may encompass "SGGQQ," "GAGQQ," "GQGPY," "AGQQ" or "SQ," wherein some positions may be absent
  VARIANT 1413..1417: note = MISC_FEATURE - This region may encompass "SGGQQ," "GAGQQ," "GQGPY," "AGQQ" or "SQ," wherein some positions may be absent
  VARIANT 1354..1417: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 1421..1440: note = MISC_FEATURE - This region may encompass 6-20 residues
  VARIANT 1447..1451: note = MISC_FEATURE - This region may encompass "SGGQQ," "GAGQQ," "GQGPY," "AGQQ" or "SQ," wherein some positions may be absent
  VARIANT 1455..1459: note = MISC_FEATURE - This region may encompass "SGGQQ," "GAGQQ," "GQGPY," "AGQQ" or "SQ," wherein some positions may be absent
  VARIANT 1463..1467: note = MISC_FEATURE - This region may encompass "SGGQQ," "GAGQQ," "GQGPY," "AGQQ" or "SQ," wherein some positions may be absent
  VARIANT 1471..1475: note = MISC_FEATURE - This region may encompass "SGGQQ," "GAGQQ," "GQGPY," "AGQQ" or "SQ," wherein some positions may be absent
  VARIANT 1479..1483: note = MISC_FEATURE - This region may encompass "SGGQQ," "GAGQQ," "GQGPY," "AGQQ" or "SQ," wherein some positions may be absent
  VARIANT 1487..1491: note = MISC_FEATURE - This region may encompass "SGGQQ," "GAGQQ," "GQGPY," "AGQQ" or "SQ," wherein some positions may be absent
  VARIANT 1495..1499: note = MISC_FEATURE - This region may encompass "SGGQQ," "GAGQQ," "GQGPY," "AGQQ" or "SQ," wherein some positions may be absent
  VARIANT 1503..1507: note = MISC_FEATURE - This region may encompass "SGGQQ," "GAGQQ," "GQGPY," "AGQQ" or "SQ," wherein some positions may be absent
  VARIANT 1444..1507: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 1511..1530: note = MISC_FEATURE - This region may encompass 6-20 residues
  VARIANT 1537..1541: note = MISC_FEATURE - This region may encompass "SGGQQ," "GAGQQ," "GQGPY," "AGQQ" or "SQ," wherein some positions may be absent
  VARIANT 1545..1549: note = MISC_FEATURE - This region may encompass "SGGQQ," "GAGQQ," "GQGPY," "AGQQ" or "SQ," wherein some positions may be absent
  VARIANT 1553..1557: note = MISC_FEATURE - This region may encompass "SGGQQ," "GAGQQ," "GQGPY," "AGQQ" or "SQ," wherein some positions may be absent
  VARIANT 1561..1565: note = MISC_FEATURE - This region may encompass "SGGQQ," "GAGQQ," "GQGPY," "AGQQ" or "SQ," wherein some positions may be absent
  VARIANT 1569..1573: note = MISC_FEATURE - This region may encompass "SGGQQ," "GAGQQ," "GQGPY," "AGQQ" or "SQ," wherein some positions may be absent
  VARIANT 1577..1581: note = MISC_FEATURE - This region may encompass "SGGQQ," "GAGQQ," "GQGPY," "AGQQ" or "SQ," wherein some positions may be absent
  VARIANT 1585..1589: note = MISC_FEATURE - This region may encompass "SGGQQ," "GAGQQ," "GQGPY," "AGQQ" or "SQ," wherein some positions may be absent
  VARIANT 1593..1597: note = MISC_FEATURE - This region may encompass "SGGQQ," "GAGQQ," "GQGPY," "AGQQ" or "SQ," wherein some positions may be absent
  VARIANT 1534..1597: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 1601..1620: note = MISC_FEATURE - This region may encompass 6-20 residues
  VARIANT 1627..1631: note = MISC_FEATURE - This region may encompass "SGGQQ," "GAGQQ," "GQGPY," "AGQQ" or "SQ," wherein some positions may be absent
  VARIANT 1635..1639: note = MISC_FEATURE - This region may encompass "SGGQQ," "GAGQQ," "GQGPY," "AGQQ" or "SQ," wherein some positions may be absent
  VARIANT 1643..1647: note = MISC_FEATURE - This region may encompass "SGGQQ," "GAGQQ," "GQGPY," "AGQQ" or "SQ," wherein some positions may be absent
  VARIANT 1651..1655: note = MISC_FEATURE - This region may encompass "SGGQQ," "GAGQQ," "GQGPY," "AGQQ" or "SQ," wherein some positions may be absent
  VARIANT 1659..1663: note = MISC_FEATURE - This region may encompass "SGGQQ," "GAGQQ," "GQGPY," "AGQQ" or "SQ," wherein some positions may be absent
  VARIANT 1667..1671: note = MISC_FEATURE - This region may encompass "SGGQQ," "GAGQQ," "GQGPY," "AGQQ" or "SQ," wherein some positions may be absent
  VARIANT 1675..1679: note = MISC_FEATURE - This region may encompass "SGGQQ," "GAGQQ," "GQGPY," "AGQQ" or "SQ," wherein some positions may be absent
  VARIANT 1683..1687: note = MISC_FEATURE - This region may encompass "SGGQQ," "GAGQQ," "GQGPY," "AGQQ" or "SQ," wherein some positions may be absent
  VARIANT 1624..1687: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 1691..1710: note = MISC_FEATURE - This region may encompass 6-20 residues
  VARIANT 1717..1721: note = MISC_FEATURE - This region may encompass "SGGQQ," "GAGQQ," "GQGPY," "AGQQ" or "SQ," wherein some positions may be absent
  VARIANT 1725..1729: note = MISC_FEATURE - This region may encompass "SGGQQ," "GAGQQ," "GQGPY," "AGQQ" or "SQ," wherein some positions may be absent
  VARIANT 1733..1737: note = MISC_FEATURE - This region may encompass "SGGQQ," "GAGQQ," "GQGPY," "AGQQ" or "SQ," wherein some positions may be absent
  VARIANT 1741..1745: note = MISC_FEATURE - This region may encompass "SGGQQ," "GAGQQ," "GQGPY," "AGQQ" or "SQ," wherein some positions may be absent
  VARIANT 1749..1753: note = MISC_FEATURE - This region may encompass "SGGQQ," "GAGQQ," "GQGPY," "AGQQ" or "SQ," wherein some positions may be absent
  VARIANT 1757..1761: note = MISC_FEATURE - This region may encompass "SGGQQ," "GAGQQ," "GQGPY," "AGQQ" or "SQ," wherein some positions may be absent
  VARIANT 1765..1769: note = MISC_FEATURE - This region may encompass "SGGQQ," "GAGQQ," "GQGPY," "AGQQ" or "SQ," wherein some positions may be absent
  VARIANT 1773..1777: note = MISC_FEATURE - This region may encompass "SGGQQ," "GAGQQ," "GQGPY," "AGQQ" or "SQ," wherein some positions may be absent
  VARIANT 1714..1777: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  VARIANT 1781..1800: note = MISC_FEATURE - This region may encompass 6-20 residues
  VARIANT 1..1800: note = MISC_FEATURE - This sequence may encompass 2-20 "GGY-[GPG-X1]n1-GPS-(A)n2" repeating units, wherein X1 is "SGGQQ," "GAGQQ," "GQGPY," "AGQQ" or "SQ," n1 is 4-8 and n2 is 6-20 and some positions may be absent
GGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAA

SEQ ID NO: 14 - protein, 5 aa, synthetic construct
  VARIANT 1..5: note = Description of Artificial Sequence: Synthetic peptide
SGGQQ

SEQ ID NO: 15 - protein, 5 aa, synthetic construct
  VARIANT 1..5: note = Description of Artificial Sequence: Synthetic peptide
GAGQQ

SEQ ID NO: 16 - protein, 5 aa, synthetic construct
  VARIANT 1..5: note = Description of Artificial Sequence: Synthetic peptide
GQGPY

SEQ ID NO: 17 - protein, 4 aa, synthetic construct
  VARIANT 1..4: note = Description of Artificial Sequence: Synthetic peptide
AGQQ

SEQ ID NO: 18 - protein, 70 aa, synthetic construct
  VARIANT 1..70: note = Description of Artificial Sequence: Synthetic polypeptide
  VARIANT 7..11: note = MISC_FEATURE - This region may encompass "SGGQQ," "GAGQQ," "GQGPY," "AGQQ" or "SQ," wherein some positions may be absent
  VARIANT 15..19: note = MISC_FEATURE - This region may encompass "SGGQQ," "GAGQQ," "GQGPY," "AGQQ" or "SQ," wherein some positions may be absent
  VARIANT 23..27: note = MISC_FEATURE - This region may encompass "SGGQQ," "GAGQQ," "GQGPY," "AGQQ" or "SQ," wherein some positions may be absent
  VARIANT 31..35: note = MISC_FEATURE - This region may encompass "SGGQQ," "GAGQQ," "GQGPY," "AGQQ" or "SQ," wherein some positions may be absent
  VARIANT 39..43: note = MISC_FEATURE - This region may encompass "SGGQQ," "GAGQQ," "GQGPY," "AGQQ" or "SQ," wherein some positions may be absent
  VARIANT 47..51: note = MISC_FEATURE - This region may encompass "SGGQQ," "GAGQQ," "GQGPY," "AGQQ" or "SQ," wherein some positions may be absent
  VARIANT 55..59: note = MISC_FEATURE - This region may encompass "SGGQQ," "GAGQQ," "GQGPY," "AGQQ" or "SQ," wherein some positions may be absent
  VARIANT 63..67: note = MISC_FEATURE - This region may encompass "SGGQQ," "GAGQQ," "GQGPY," "AGQQ" or "SQ," wherein some positions may be absent
  VARIANT 4..67: note = MISC_FEATURE - This region may encompass 4-8 "GPG-X1" repeating units, wherein X1 is "SGGQQ," "GAGQQ," "GQGPY," "AGQQ" or "SQ," and some positions may be absent
GGYGPGXXXXXGPGXXXXXGPGXXXXXGPGXXXXXGPGXXXXXGPGXXXXXGPGXXXXXGPGXXXXXGPS

SEQ ID NO: 19 - protein, 20 aa, synthetic construct
  VARIANT 1..20: note = Description of Artificial Sequence: Synthetic peptide
  VARIANT 1..20: note = MISC_FEATURE - This sequence may encompass 6-20 residues
AAAAAAAAAAAAAAAAAAAA

SEQ ID NO: 20 - DNA, 2835 bp, synthetic construct
  misc_feature 1..2835: note = Description of Artificial Sequence: Synthetic polynucleotide
  source 1..2835: mol_type = other DNA
ggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgca

SEQ ID NO: 21 - protein, 945 aa, synthetic construct
  VARIANT 1..945: note = Description of Artificial Sequence: Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 22 - protein, 315 aa, synthetic construct
  VARIANT 1..315: note = Description of Artificial Sequence: Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 23 - protein, 5 aa, synthetic construct
  VARIANT 1..5: note = Description of Artificial Sequence: Synthetic peptide
SGAGG

SEQ ID NO: 24 - protein, 5 aa, synthetic construct
  VARIANT 1..5: note = Description of Artificial Sequence: Synthetic peptide
GSGAG

SEQ ID NO: 25 - protein, 5 aa, synthetic construct
  VARIANT 1..5: note = Description of Artificial Sequence: Synthetic peptide
GGSGA

SEQ ID NO: 26 - protein, 181 aa, Aliatypus gulosus
GAASSSSTIITTKSASASAAADASAAATASAASRSSANAAASAFAQSFSSILLESGYFCSIFGSSISSSYAAAIASAASRAAAESNGYTTHAYACAKAVASAVERVTSGADAYAYAQAISDALSHALLYTGRLNTANANSLASAFAYAFANAAAQASASSASAGAASASGAASASGAGSAS

SEQ ID NO: 27 - protein, 126 aa, Plectreurys tristis
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 28 - protein, 239 aa, Plectreurys tristis
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 29 - protein, 182 aa, Araneus gemmoides
GNVGYQLGLKVANSLGLGNAQALASSLSQAVSAVGVGASSNAYANAVSNAVGQVLAGQGILNAANAGSLASSFASALSSSAASVASQSASQSQAASQSQAAASAFRQAASQSASQSDSRAGSQSSTKTTSTSTSGSQADSRSASSSASQASASAFAQQSSASLSSSSSFSSAFSSATSISAV

SEQ ID NO: 30 - protein, 180 aa, Argiope aurantia
GSLASSFASALSASAASVASSAAAQAASQSQAAASAFSRAASQSASQSAARSGAQSISTTTTTSTAGSQAASQSASSAASQASASSFARASSASLAASSSFSSAFSSANSLSALGNVGYQLGFNVANNLGIGNAAGLGNALSQAVSSVGVGASSSTYANAVSNAVGQFLAGQGILNAANA

SEQ ID NO: 31 - protein, 199 aa, Deinopis spinosa
GASASAYASAISNAVGPYLYGLGLFNQANAASFASSFASAVSSAVASASASAASSAYAQSAAAQAQAASSAFSQAAAQSAAAASAGASAGAGASAGAGAVAGAGAVAGAGAVAGASAAAASQAAASSSASAVASAFAQSASYALASSSAFANAFASATSAGYLGSLAYQLGLTTAYNLGLSNAQAFASTLSQAVTGVGL

SEQ ID NO: 32 - protein, 171 aa, Nephila clavipes
GATAASYGNALSTAAAQFFATAGLLNAGNASALASSFARAFSASAESQSFAQSQAFQQASAFQQAASRSASQSAAEAGSTSSSTTTTTSAARSQAASQSASSSYSSAFAQAASSSLATSSALSRAFSSVSSASAASSLAYSIGLSAARSLGIADAAGLAGVLARAAGALGQ

SEQ ID NO: 33 - protein, 268 aa, Argiope trifasciata
GGAPGGGPGGAGPGGAGFGPGGGAGFGPGGGAGFGPGGAAGGPGGPGGPGGPGGAGGYGPGGAGGYGPGGVGPGGAGGYGPGGAGGYGPGGSGPGGAGPGGAGGEGPVTVDVDVTVGPEGVGGGPGGAGPGGAGFGPGGGAGFGPGGAPGAPGGPGGPGGPGGPGGPGGVGPGGAGGYGPGGAGGVGPAGTGGFGPGGAGGFGPGGAGGFGPGGAGGFGPAGAGGYGPGGVGPGGAGGFGPGGVGPGGSGPGGAGGEGPVTVDVDVSV

SEQ ID NO: 34 - protein, 420 aa, Nephila clavipes
GVSYGPGGAGGPYGPGGPYGPGGEGPGGAGGPYGPGGVGPGGSGPGGYGPGGAGPGGYGPGGSGPGGYGPGGSGPGGYGPGGSGPGGYGPGGSGPGGYGPGGYGPGGSGPGGSGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAAPGGAGPGGAGPGGAGPGGAGPGGAGPGGAGPGGAGGAGGAGGSGGAGGSGGTTIIEDLDITIDGADGPITISEELPISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGP

SEQ ID NO: 35 - protein, 376 aa, Latrodectus hesperus
GINVDSDIGSVTSLILSGSTLQMTIPAGGDDLSGGYPGGFPAGAQPSGGAPVDFGGPSAGGDVAAKLARSLASTLASSGVFRAAFNSRVSTPVAVQLTDALVQKIASNLGLDYATASKLRKASQAVSKVRMGSDTNAYALAISSALAEVLSSSGKVADANINQIAPQLASGIVLGVSTTAPQFGVDLSSINVNLDISNVARNMQASIQGGPAPITAEGPDFGAGYPGGAPTDLSGLDMGAPSDGSRGGDATAKLLQALVPALLKSDVFRAIYKRGTRKQVVQYVTNSALQQAASSLGLDASTISQLQTKATQALSSVSADSDSTAYAKAFGLAIAQVLGTSGQVNDANVNQIGAKLATGILRGSSAVAPRLGIDLS

SEQ ID NO: 36 - protein, 200 aa, Argiope trifasciata
GAGYTGPSGPSTGPSGYPGPLGGGAPFGQSGFGGSAGPQGGFGATGGASAGLISRVANALANTSTLRTVLRTGVSQQIASSVVQRAAQSLASTLGVDGNNLARFAVQAVSRLPAGSDTSAYAQAFSSALFNAGVLNASNIDTLGSRVLSALLNGVSSAAQGLGINVDSGSVQSDISSSSSFLSTSSSSASYSQASASSTS

SEQ ID NO: 37 - protein, 357 aa, Uloborus diversus
GASAADIATAIAASVATSLQSNGVLTASNVSQLSNQLASYVSSGLSSTASSLGIQLGASLGAGFGASAGLSASTDISSSVEATSASTLSSSASSTSVVSSINAQLVPALAQTAVLNAAFSNINTQNAIRIAELLTQQVGRQYGLSGSDVATASSQIRSALYSVQQGSASSAYVSAIVGPLITALSSRGVVNASNSSQIASSLATAILQFTANVAPQFGISIPTSAVQSDLSTISQSLTAISSQTSSSVDSSTSAFGGISGPSGPSPYGPQPSGPTFGPGPSLSGLTGFTATFASSFKSTLASSTQFQLIAQSNLDVQTRSSLISKVLINALSSLGISASVASSIAASSSQSLLSVSA

SEQ ID NO: 38 - protein, 32 aa, Euprosthenops australis
GGQGGQGQGRYGQGAGSSAAAAAAAAAAAAAA

SEQ ID NO: 39 - protein, 42 aa, Tetragnatha kauaiensis
GGLGGGQGAGQGGQQGAGQGGYGSGLGGAGQGASAAAAAAAA

SEQ ID NO: 40 - protein, 42 aa, Argiope aurantia
GGYGPGAGQQGPGSQGPGSGGQQGPGGLGPYGPSAAAAAAAA

SEQ ID NO: 41 - protein, 46 aa, Deinopis spinosa
GPGGYGGPGQQGPGQGQYGPGTGQQGQGPSGQQGPAGAAAAAAAAA

SEQ ID NO: 42 - protein, 42 aa, Nephila clavata
GPGGYGLGQQGPGQQGPGQQGPAGYGPSGLSGPGGAAAAAAA

SEQ ID NO: 43 - protein, 3360 aa, Argiope bruennichi
MNWSIRLALLGFVVLSTQTVFAVGQAATPWENSQLAEDFINSFLRFIAQSGAFSPNQLDDMSSIGDTLKTAIEKMAQSRKSSKSKLQALNMAFASSMAEIAVAEQGGLSLEAKTNAIANALASAFLETTGFVNQQFVSEIKSLIYMIAQASSNEISGSAAAAGGGSGGGGGSGQGGYGQGASASASAAAAYGSAPQGAGGPAPQGPSQQGPVSQGPYGPGAAAAAAAAGGYGPGAGQQGPGSQGPGSGGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGPGAGRQGPGSQGPGSGGQQGPGGQGPYGPSAAAAAAAAGPGARRQGPGSQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPEGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGGGYGPGAGQQGPGSQGPGSGGQQGPGGQGPYGPSAAAAAAAAGPGARRQGPGSQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGPGAGRQGPGSQGPGSGGQQGPGGQGPYGPSAAAAAAAAGPGARRQGPGSQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGSQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGSQGPGSGGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPSSQAPVASAAASRLSSPQASARVSSAVSTLVSSGPTSPAALSNAISSVVSQVSASNPGLSGCDVLVQALLEIVSALVHILGSSSIGQINYAASSQYAQMVGNSVAQALG

SEQ ID NO: 44 - protein, 3129 aa, Latrodectus hesperus
MTWSTRLALSFLFVLCTQSLYALAQANTPWSSKANADAFINSFISAASNTGSFSQDQMEDMSLIGNTLMAAMDNMGGRITPSKLQALDMAFASSVAEIAASEGGDLGVTTNAIADALTSAFYQTTGVVNSRFISEIRSLIGMFAQASANDVYASAGSSGGGGYGASSASAASASAAAPSGVAYQAPAQAQISFTLRGQQPVSYGQGGAGPGGAGAAAAAAAAAGGAGQGGQGGYGQGGYGQGGAGQGGSGAAAAAAAAAGGTGQGGAGQGGAGAAAAAAAAAGGAGQGGQGGYGQGGYGQGGTGQGGAGAAAAAAAAGGAGQGGQGGYGQGGYGQGGYGQGGSGAAAAAAAAAGGAGQGGQGGYGQGGYGQGGAGQGGAGAAAAAAAAAGGAGQGGYGRGGAGQGGAAAAAAAAAGAGQGGYGGQGAGQGGSGAAAAAAAAGGAGQGGQGGYGQGGYGQGGSGAAAAAAAAGGAGQGGQGGYGQGGYGQGGAGQGGAGAAAAAAAAGGAGQGGQGGYGQGGYGQGGAGQGGAGAAAAAAAAGGAGQGGQGGYGQGGYGQGGAGQGGAGAAAAAAAAGGAGQGGQGGYGQGGYGQGGAGQGGAAAAAAAAAGGAGQGGYGRGGAGQGGAAAAAGAGQGGYGGQGAGQGGAGAAAAAAAAGGAGQGGQGGYGRGGYGQGGAGQGGAGAAAAAAAAGGAGQGGQGGYGQGGYGQGGAGQGGAAAAAAAAGGAGQGGYGRGGAGQGGAAAAAAAAAGAGQGGYGGQGAGQGGAGAAAAAAAAGGAGQGGQGDYGRGGYGQGGAGQGGAGAAAAAAAAGGAGQGGQGGYGQGGYGQGGAGQGGAAAAASAAAAGGAGQGGYGRGGAGQGGAAAAAGAGQGGYGGQGAGQGGAGAAAAAAAAGGAGQGGQGGYGRGGYGQGGAGQGGAGAAAAATAAGGAGQGGQGGYGQGGYGQGGAGQGGAAAAAAAAAGGAGQGGYGRGGAGQGGAAAAAAAAAGAGQGGYGGQGAGQGGAGAAAAAAGGAGQGGQGGYGRGGYGQGGAGQGGAGAAAAAAAAGGAGQGGQGGYGQGGYGQGGAGQGGAAAAAAAAAGGAGQGGYGRGGAGQGGAAAAAGAGQGGYGGQGAGQGGAGAAAAASRGAGQGGQGGYGRGGYGQGGAGQGGAGAAAAAAAAGGAGQGGQGGYGQGGYGQGGAGQGGAAAAAAAAGGAGQGGYGRGGAGQGGAAAAAGAGQGGYGGQGAGQGGAGAAAAAAAAGGAGQGGQGGYGRGGYGQGGAGQGGAGAAAAAAAAGGAGQGGQGGYGQGGYGQGGAGQGGAAAAAAAAAGGAGQGGYGRGGAGQGGAAAAAAAAAGSGQGGYGGQGAGQGGAGAAAAAAAAGGAGQGGQGGYGRGGYGQGGAGQGGAGAAAAAAAAGGAGQGGQGGYGQGGYGQGGAGQGGAAAAAAAAAAGGAGQGGYGRGGAGQGGAAAAAGAGQGGYGGQGAGQGGAGAAAAAAAAGGAGQGGQGGYGRGGYGQGGAGQGGAGTAAAAAAAGGAGQGGQGGYGQGGYGQGGAGQGGAAAAAAAAAGGAGQGGYGRGGAGQGGAAAAAAAAAGAGQGGYGGQGAGQGGAGAAAAAAAAGGAGQGGQGGYGRGGYGQGGAGQGGAGAAAAAAAAGGASQGGQGGYGQGDYGQGGAGQGGAAAAAAAAGGAGQGGYGRGGAGQGGAAAAAGAGQGGYGGQGAGQGGAGAAAAAAAAGGAGRGGQGGYGRGGYGQGGAGQGGAGAAAAAAAAGGAGQGGQGGYGQGGYGQGGTGQGGAAAAAAAAAGGAGQGGYGRGGAGQGGAAAAAAAAAGAGQGGYGGQGAGQGGAGAAAAAAAAGGAGQGGQGGYGRGGYGQGGAGQGGAGAAAAAAAAGGAGQGGQGGYGQGGYGQGGYGQGGAGQGGAAAAAAAAGGAGQGGYGRGGAGQGGAAAAAGAGQGGYGGQGAGQGGAGAAAAAAAAGGAGQGGQGGYGRGGYGQGGAGQGGAGAAAAAAAAGGAGQGGQGGYGQGGYGQGGAGQGGAAAAAAAAAGGAGQGGYGRGGAGQGGAAAAAAAAAGSGQGGYGGQGAGQGGAGAAAAAAAAGGAGQGGQGGYGRGGYGQGGAGQGGAGAAAAAAAAGGAGQGGQGGYGQGGYGQGGYGQGGAGQGGAAAAAAAAAAGGAGQGGYGRGGAGQGGAAAAAGAGQGGYGGQGAGQGGAGAAAAAAAAGGAGQGGQGGYGRGGYGQGGAGQGGAGAAAAAAAAGGAGQGGQGGYGQGGNGQGGAGQGGAAAAAAAAGGAGQGGYGRGGAGQGGAAAAAAAAAGAGQGGYGGQGAGQGGAGAAAAAAAAGGAGQGGQGGYGRGGYGQGGAGQGGAGAAAAAAAAGGASQGGQGGYGQGDYGQGGAGQGGAAAAAAAAGGAGQGGYGRGGAGQGGAAAAAGAGQGGYGGQGAGQGGAGAAAAAAAAGGAGRGGQGGYGRGGYGQGGAGQGGAGAAAAAAAAGGAGQGGQGGYGQGGYGQGGAGQGGAAAAAAAAAGGAGQGGYGRGGAGQGGAAAAAGAGQGGYGGQGAGQGGAGAAAAAAAAGGAGRGGQGGYGRGGYGQGGAGQGGAGAAAAAAAAGGAGQGGQGGYGQGGYGQGGAGQGGAAAAAAAAVGGAGQGGYGRGGAGQGGAAAAAAAAAAGSGQGGYGGQGAGQGGAGAAAAAAAAGGAGQGGQGGYGGGGYGQGGAGQGGAGAAAAAAAAGGAGQGGQGGYGQGGYGQGGAGQGGAAAAAAAAAGGAGQGGYGRGGAGQGGAAAATGAGQGGYGGQGAGQGGAGAAAAAAAAGGAGQGGQGGYGRGGYGQGGAGQGGAGAAAAAAAAGGAGQGGQGGYGQGGYGQGGAGQGGAAAAAAAAGGAGQGGYGRGGAGQGGAAAAAAAAAGAGQGGYGGQGAGQGGAGAAAAAAAAGGAGQGGQGGYGRGGYGQGGAGQGGAGAAAAGGAGQGGQGGYGQGGYGQGGAGQGGAAAAAAAAAAGGAGQGGYGGYGQQGGAGAAAAAASGPGQIYYGPQSVAAPAAAAASALAAPATSARISSHASALLSNGPTNPASISNVISNAVSQISSSNPGASACDVLVQALLELVTALLTIIGSSNIGSVNYDSSGQYAQVVTQSVQNAFA

SEQ ID NO: 45 - DNA, 20 bp, synthetic construct
  misc_feature 1..20: note = Description of Artificial Sequence: Synthetic primer
  source 1..20: mol_type = other DNA
tactacaggctggctgttcc

SEQ ID NO: 46 - DNA, 46 bp, synthetic construct
  misc_feature 1..46: note = Description of Artificial Sequence: Synthetic primer
  source 1..46: mol_type = other DNA
ctcacttaatcttctgtactctgaagaagtccaactgttgaacgcc

SEQ ID NO: 47 - DNA, 46 bp, synthetic construct
  misc_feature 1..46: note = Description of Artificial Sequence: Synthetic primer
  source 1..46: mol_type = other DNA
agaagttgattgagactttcaacgagggtccccttcagctaccttt

SEQ ID NO: 48 - DNA, 20 bp, synthetic construct
  misc_feature 1..20: note = Description of Artificial Sequence: Synthetic primer
  source 1..20: mol_type = other DNA
tccctgctaagccctaatcg

SEQ ID NO: 49 - DNA, 20 bp, synthetic construct
  misc_feature 1..20: note = Description of Artificial Sequence: Synthetic primer
  source 1..20: mol_type = other DNA
ctctgattgcacgagaaggc

SEQ ID NO: 50 - DNA, 46 bp, synthetic construct
  misc_feature 1..46: note = Description of Artificial Sequence: Synthetic primer
  source 1..46: mol_type = other DNA
ctcacttaatcttctgtactctgaagtgaaaggcgattggagttgc

SEQ ID NO: 51 - DNA, 46 bp, synthetic construct
  misc_feature 1..46: note = Description of Artificial Sequence: Synthetic primer
  source 1..46: mol_type = other DNA
agaagttgattgagactttcaacgagctggctctgcttctggtact

SEQ ID NO: 52 - DNA, 20 bp, synthetic construct
  misc_feature 1..20: note = Description of Artificial Sequence: Synthetic primer
  source 1..20: mol_type = other DNA
gatgttgaggcgggcataag

SEQ ID NO: 53 - DNA, 20 bp, synthetic construct
  misc_feature 1..20: note = Description of Artificial Sequence: Synthetic primer
  source 1..20: mol_type = other DNA
acttgtcaggacgatacgga

SEQ ID NO: 54 - DNA, 20 bp, synthetic construct
  misc_feature 1..20: note = Description of Artificial Sequence: Synthetic primer
  source 1..20: mol_type = other DNA
ccggtctccctggaaataga

SEQ ID NO: 55 - DNA, 20 bp, synthetic construct
  misc_feature 1..20: note = Description of Artificial Sequence: Synthetic primer
  source 1..20: mol_type = other DNA
agttgtccgtcattagccct

SEQ ID NO: 56 - DNA, 20 bp, synthetic construct
  misc_feature 1..20: note = Description of Artificial Sequence: Synthetic primer
  source 1..20: mol_type = other DNA
tgttccctttcggctagaca

SEQ ID NO: 57 - DNA, 4324 bp, synthetic construct
  misc_feature 1..4324: note = Description of Artificial Sequence: Synthetic polynucleotide
  source 1..4324: mol_type = other DNA
ggagttgaatcacatcttactggatagcgagctttttgacgaagtgaaaatttctaattttaaacaagaggaaggggtcaaaaacggagatatcttatacttggaaaaagagatgacaatcagtgatttcatcaattttgtatctagttggccttctgtgttttcgtggaagcagcaacgaggaaaggagggtatcctagatgatttttacaacgaactgaacgactgctttgaggggggtaacatgaaagtaatatggaactccgtcctagtatttgccaggaggaagcaaagggttgtataggctttagtacttatagaggaaacggggttacgtgcaagcgcgcatgcctgagctttgaggggggggactttcacatctcttcttctcacacttagccctaacacagagaataataaaaagcattgcaagatgagtgttgtcagcaagcaatacgacatccacgaaggcattatctttgtaattgaattgaccccggagcttcacgcgccggcttcagaagggaaatctcagctccagatcatcttagagaatgtcagtgaggttatttctgagctaatcattaccttgcccggtacaggaatagggtgttaccttattaattacgacggtggtcaaaacgacgaaatttaccccatttttgagttacaagacctgaatttggaaatgatgaaacaattgtaccaagtcttggaggaccatgtaagtgggcttaatcctctcgagaagcaattcccaattgaacacagtaaaccgttatcagccactctgttctttcacttaaggtctcttttttacatggcgaagactcataagcgtactggaagacattacaacttgaaaaagattttcttgttcactaataacgataaaccttacaatggaaactctcagctgagagttcccttgaagaaaaccctggctgattacaatgacgtagacattactttgattccgtttcttctgaacaagccttcaggtgtcaagtttgacaagacggaatactcagaaattttgttctatgataaagatgcttgttcgatgtcaattgaggagatccgccaacgaatttctagacataaggagatcaagcgggtttacttcacctgtcctttgaaaatcgcaaataacttgtgcatttctgtgaaaggttattctatgttttatcatgaaactccaaggaagatcaaatttgtcgtcaatgagggttcaactttcaaagatgtggagacaaaatctcagtttgtcgatccaacatccggaaaagagttttccagtgaacagctgatcaaagcatatcctctaggtgccgatgcttacattcctttaaactcagagcaagtcaaaacaataaatcgatttaatgatatcatcaatatcccctctttggaaattctaggtttcagggatatatctaattggttgccacagtatcagtttggcaaagcatcgtttttatcccctaataactatggtgattttacacattcgcagagaacatttagttgtcttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatggctaaactgacctctgctgttccggttctgaccgctcgtgacgttgctggtgctgttgagttctggaccgaccgtctgggtttctctcgtgacttcgttgaagacgacttcgctggtgttgttcgtgacgacgttaccctgttcatctctgctgttcaggaccaggttgttccggacaacaccctggcttgggtttgggttcgtggtctggacgaactgtacgctgaatggtctgaagttgtttctaccaacttccgtgacgcttctggtccggctatgaccgaaatcggtgaacagccgtggggtcgtgagttcgctctgcgtgacccggctggtaactgcgttcacttcgttgctgaagaacaggactaacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgtattagtttcacttttcagcaacctggtcggaaagatccacatcaagaatggataccaaccccaagagtatgaaaatccttccctacaatggcacttcaaaatgttacgtgacgattaccttcaattggaacacgatatcgacatcagtgacccccttgagaaacaaaagtacataaacagcctcgatgagacaaaaaccaagatcatgaaactacgggactatgtcaaggaaactgccgatgatgacgacccttcacggcttgccaacactctcaaagagctcaaccaagagctgaacaaaatttccaactttgatatcatcgccaataagaagccaaagacccccacgacagtagaccctgttcctactgatgatgacatcatcaacgcctggaaggcaggaactctgaacggtttcaaggtggatcaattacgaaaatacgtaaggtcacgaaacaactttctggagacggcctccaaaaaggcagatctcatcgccaacattgacaagtactttcagcagaagttcaaagagactaaggcctgattcgtgttccttactttttcctcgcaacgtgtttttttcccaccacattgcctatgttgtaatgcaatgcagatgctggcccagtttttgacgattctcgaaaattggcattttcgtcgatgccattggccaaactgaaaattcaagacaaaatagattggattttatctgcaacgtcttccacctacacaaccactctacaaacttcagacaaacatgtttataaaagcagctactagatccaaaatgacaagttcgttattctctactacgtttgttgtggcatttggattggtggctagcaacaacctcttgccatgtcctgttgaccactctatgaataacgagactccgcaagaattgaaaccattgcaggctgaatcttctactagaaagttgaactcttccgcttaagtcaaataaaactactgacacagatgatgcacagaaacaacggatcacgctcttgactgattagtcccgtcattttggttctcattttcttcacagtcacctatcaatgtatgatcacctggaaggatttccctacgatacttcaaatcttttacttgataatattactcattatggctcaggaatgcagactgcctgattcaagacgctgctcttcttatttaacacttgtacactaaccccatggaagccagggaagggaataaccatctctctggtaataaatcggtctttatttatgcatagaaaaggaatctattatatttcgttcatttggcactctgctaactgtagattaacgggtctcgtaaattcaaaatcttcttccgatcaaaccggggtgaaatattacttctcgtgcatagctaattttcaaataaccgtcctaaaatgaacggtcatttacctggactctcttgccaaatgggcaacaaaacataaagctgatcagaacgtaactagtctctcggaatccat

SEQ ID NO: 58 - DNA, 22 bp, synthetic construct
  misc_feature 1..22: note = Description of Artificial Sequence: Synthetic oligonucleotide
  source 1..22: mol_type = other DNA
ggagttgaatcacatcttactg

SEQ ID NO: 59 - DNA, 1099 bp, synthetic construct
  misc_feature 1..1099: note = Description of Artificial Sequence: Synthetic polynucleotide
  source 1..1099: mol_type = other DNA
gacaactaaatgttctctgcgaatgtgtaaaatcaccatagttattaggggataaaaacgatgctttgccaaactgatactgtggcaaccaattagatatatccctgaaacctagaatttccaaagaggggatattgatgatatcattaaatcgatttattgttttgacttgctctgagtttaaaggaatgtaagcatcggcacctagaggatatgctttgatcagctgttcactggaaaactcttttccggatgttggatcgacaaactgagattttgtctccacatctttgaaagttgaaccctcattgacgacaaatttgatcttccttggagtttcatgataaaacatagaataacctttcacagaaatgcacaagttatttgcgattttcaaaggacaggtgaagtaaacccgcttgatctccttatgtctagaaattcgttggcggatctcctcaattgacatcgaacaagcatctttatcatagaacaaaatttctgagtattccgtcttgtcaaacttgacacctgaaggcttgttcagaagaaacggaatcaaagtaatgtctacgtcattgtaatcagccagggttttcttcaagggaactctcagctgagagtttccattgtaaggtttatcgttattagtgaacaagaaaatctttttcaagttgtaatgtcttccagtacgcttatgagtcttcgccatgtaaaaaagagaccttaagtgaaagaacagagtggctgataacggtttactgtgttcaattgggaattgcttctcgagaggattaagcccacttacatggtcctccaagacttggtacaattgtttcatcatttccaaattcaggtcttgtaactcaaaaatggggtaaatttcgtcgttttgaccaccgtcgtaattaataaggtaacaccctattcctgtaccgggcaaggtaatgattagctcagaaataacctcactgacattctctaagatgatctggagctgagatttcccttctgaagccggcgcgtgaagctccggggtcaattcaattacaaagataatgccttcgtggatgtcgtattgcttgctgacaacactcat

SEQ ID NO: 60 - DNA, 549 bp, synthetic construct
  misc_feature 1..549: note = Description of Artificial Sequence: Synthetic polynucleotide
  source 1..549: mol_type = other DNA
tcaggccttagtctctttgaacttctgctgaaagtacttgtcaatgttggcgatgagatctgcctttttggaggccgtctccagaaagttgtttcgtgaccttacgtattttcgtaattgatccaccttgaaaccgttcagagttcctgccttccaggcgttgatgatgtcatcatcagtaggaacagggtctactgtcgtgggggtctttggcttcttattggcgatgatatcaaagttggaaattttgttcagctcttggttgagctctttgagagtgttggcaagccgtgaagggtcgtcatcatcggcagtttccttgacatagtcccgtagtttcatgatcttggtttttgtctcatcgaggctgtttatgtacttttgtttctcaagggggtcactgatgtcgatatcgtgttccaattgaaggtaatcgtcacgtaacattttgaagtgccattgtagggaaggattttcatactcttggggttggtatccattcttgatgtggatctttccgaccaggttgctgaaaagtgaaactaatac

SEQ ID NO: 61 - DNA, 552 bp, synthetic construct
  misc_feature 1..552: note = Description of Artificial Sequence: Synthetic polynucleotide
  source 1..552: mol_type = other DNA
ttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattt

SEQ ID NO: 62 - DNA, 20 bp, synthetic construct
  misc_feature 1..20: note = Description of Artificial Sequence: Synthetic oligonucleotide
  source 1..20: mol_type = other DNA
cagaggccaaacattccacc

SEQ ID NO: 63 - DNA, 14 bp, synthetic construct
  misc_feature 1..14: note = Description of Artificial Sequence: Synthetic oligonucleotide
  source 1..14: mol_type = other DNA
ttaaagaggagaaa

SEQ ID NO: 64 - DNA, 375 bp, synthetic construct
  misc_feature 1..375: note = Description of Artificial Sequence: Synthetic polynucleotide
  source 1..375: mol_type = other DNA
atggctaaactgacctctgctgttccggttctgaccgctcgtgacgttgctggtgctgttgagttctggaccgaccgtctgggtttctctcgtgacttcgttgaagacgacttcgctggtgttgttcgtgacgacgttaccctgttcatctctgctgttcaggaccaggttgttccggacaacaccctggcttgggtttgggttcgtggtctggacgaactgtacgctgaatggtctgaagttgtttctaccaacttccgtgacgcttctggtccggctatgaccgaaatcggtgaacagccgtggggtcgtgagttcgctctgcgtgacccggctggtaactgcgttcacttcgttgctgaagaacaggactaa

SEQ ID NO: 65 - DNA, 318 bp, synthetic construct
  misc_feature 1..318: note = Description of Artificial Sequence: Synthetic polynucleotide
  source 1..318: mol_type = other DNA
cacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagct

SEQ ID NO: 66 - DNA, 20 bp, synthetic construct
  misc_feature 1..20: note = Description of Artificial Sequence: Synthetic oligonucleotide
  source 1..20: mol_type = other DNA
aggagttagacaacctgaag

SEQ ID NO: 67 - DNA, 23 bp, synthetic construct
  misc_feature 1..23: note = Description of Artificial Sequence: Synthetic oligonucleotide
  source 1..23: mol_type = other DNA
gtaactagtctctcggaatccat

SEQ ID NO: 68 - DNA, 1623 bp, synthetic construct
  misc_feature 1..1623: note = Description of Artificial Sequence: Synthetic polynucleotide
  source 1..1623: mol_type = other DNA
cttcagagtacagaagattaagtgagagaattctaccgttcgtatagcatacattatacgaagttatttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatgactactcttgatgacacagcctacagatataggacatcagttccgggtgacgcagaggctatcgaagccttggacggttcattcactactgatacggtgtttagagtcaccgctacaggtgatggcttcaccttgagagaggttcctgtagacccacccttaacgaaagttttccctgatgacgaatcggatgacgagtctgatgctggtgaggacggtgaccctgattccagaacatttgtcgcatacggagatgatggtgacctggctggctttgttgtggtgtcctacagcggatggaatcgtagactcacagttgaggacatcgaagttgcacctgaacatcgtggtcacggtgttggtcgtgcactgatgggactggcaacagagtttgctagagaaagaggagccggacatttgtggttagaagtgaccaatgtcaacgctcctgctattcacgcatataggcgaatgggtttcactttgtgcggtcttgatactgctttgtatgacggaactgcttctgatggtgaacaagctctttacatgagtatgccatgtccatagcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctataacttcgtatagcatacattataccttgttatgcggccgcaagaagttgattgagactttcaacgag

SEQ ID NO: 69 - DNA, 27 bp, synthetic construct
  misc_feature 1..27: note = Description of Artificial Sequence: Synthetic oligonucleotide
  source 1..27: mol_type = other DNA
cttcagagtacagaagattaagtgaga

SEQ ID NO: 70 - DNA, 34 bp, synthetic construct
  misc_feature 1..34: note = Description of Artificial Sequence: Synthetic oligonucleotide
  source 1..34: mol_type = other DNA
taccgttcgtatagcatacattatacgaagttat

SEQ ID NO: 71 - DNA, 552 bp, synthetic construct; identical to SEQ ID NO: 61
  misc_feature 1..552: note = Description of Artificial Sequence: Synthetic polynucleotide
  source 1..552: mol_type = other DNA

SEQ ID NO: 72 - DNA, 14 bp, synthetic construct
  misc_feature 1..14: note = Description of Artificial Sequence: Synthetic oligonucleotide
  source 1..14: mol_type = other DNA
ttaaagaggagaaa

SEQ ID NO: 73 - DNA, 570 bp, synthetic construct
  misc_feature 1..570: note = Description of Artificial Sequence: Synthetic polynucleotide
  source 1..570: mol_type = other DNA
atgactactcttgatgacacagcctacagatataggacatcagttccgggtgacgcagaggctatcgaagccttggacggttcattcactactgatacggtgtttagagtcaccgctacaggtgatggcttcaccttgagagaggttcctgtagacccacccttaacgaaagttttccctgatgacgaatcggatgacgagtctgatgctggtgaggacggtgaccctgattccagaacatttgtcgcatacggagatgatggtgacctggctggctttgttgtggtgtcctacagcggatggaatcgtagactcacagttgaggacatcgaagttgcacctgaacatcgtggtcacggtgttggtcgtgcactgatgggactggcaacagagtttgctagagaaagaggagccggacatttgtggttagaagtgaccaatgtcaacgctcctgctattcacgcatataggcgaatgggtttcactttgtgcggtcttgatactgctttgtatgacggaactgcttctgatggtgaacaagctctttacatgagtatgccatgtccatag

SEQ ID NO: 74 - DNA, 318 bp, synthetic construct; identical to SEQ ID NO: 65
  misc_feature 1..318: note = Description of Artificial Sequence: Synthetic polynucleotide
  source 1..318: mol_type = other DNA

SEQ ID NO: 75 - DNA, 34 bp, synthetic construct
  misc_feature 1..34: note = Description of Artificial Sequence: Synthetic oligonucleotide
  source 1..34: mol_type = other DNA
ataacttcgtatagcatacattataccttgttat

SEQ ID NO: 76 - DNA, 35 bp, synthetic construct
  misc_feature 1..35: note = Description of Artificial Sequence: Synthetic oligonucleotide
  source 1..35: mol_type = other DNA
gcggccgcaagaagttgattgagactttcaacgag

SEQ ID NO: 77 - DNA, 3869 bp, synthetic construct
  misc_feature 1..3869: note = Description of Artificial Sequence: Synthetic polynucleotide
  source 1..3869: mol_type = other DNA
tactacaggctggctgttcctcgcatggtgtttaatgtcctgactgggttttcgtttatcggtattaccggagccaccttgactgtaagggaacgatactggactaagagagtaatgcgaaaggcaacagcgtttctggcgaacctaatcaatgacggttacgagtttactactcctaaagccagtcttattttgctagagcgagtcaacgcttacttaaagggccagggacctaattatgacatcgattttgacgagcaggaggcgttcattaaagaaatggaggagttgaggacctctggtggatatgagaacagatactcatattcaggaaccgatgaaacacccagagatccgggttgcctgtttcttcccattgctttaaataaatggcactttgatgtgctagactgcctgaggatatacggtactcaggaagatctggaatctaaattattaagtgttcagcaattggtgttacaatgttgcatgaagcacagtggcatgactccagacatggtctttgcaacggaagtagctcagaagccgaccttcgaagacgacatagtttgtgatgatattgacgcttatgcccaggggggtgattgtctagattattgttacacgccaagcaattactccagaactttagaaattcatggcaagattgctaccttacaacgagagctggggctatgctataatattctcggaattttggaccgtttttccgattaaggtttttagctccattgcgccaacccccgctctccagactccttcgttatccagcattcagcatggacaggttcaaaaaataaaatttcttgatatgggtccacttcaaacatgcgcctacctgtaggaaaaaaaaagagaacataaatatgccgcgaacagaaaacgtaatgtactgttctatatataaactgttcagatcaatcataaattctcagtttcaaactttccgctcagccagattttattcgtaaagaacgcatcattggctctatgttgaaggatcagttcttgttatgggttgctttgatagcgagcgtaccggtttccggcgtgatggcagctcctagcgagtccgggcataacacggttgaaaaacgagatgccaaaaacgttgttggcgttcaacagttggacttcttcagagtacagaagattaagtgagagaattctaccgttcgtatagcatacattatacgaagttatttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatgactactcttgatgacacagcctacagatataggacatcagttccgggtgacgcagaggctatcgaagccttggacggttcattcactactgatacggtgtttagagtcaccgctacaggtgatggcttcaccttgagagaggttcctgtagacccacccttaacgaaagttttccctgatgacgaatcggatgacgagtctgatgctggtgaggacggtgaccctgattccagaacatttgtcgcatacggagatgatggtgacctggctggctttgttgtggtgtcctacagcggatggaatcgtagactcacagttgaggacatcgaagttgcacctgaacatcgtggtcacggtgttggtcgtgcactgatgggactggcaacagagtttgctagagaaagaggagccggacatttgtggttagaagtgaccaatgtcaacgctcctgctattcacgcatataggcgaatgggtttcactttgtgcggtcttgatactgctttgtatgacggaactgcttctgatggtgaacaagctctttacatgagtatgccatgtccatagcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctataacttcgtatagcatacattataccttgttatgcggccgcaagaagttgattgagactttcaacgagggtccccttcagctacctttctctctgtttggtagttattctcggcgtgtgtatagtatagtataaaagggcctacattggataggcttcaacattcctcaataaacaaacatccaacatcgcgcattccgcatttcgcatttcacatttcgcgcctgccttcctttaggttctttgaatcatcatcaatcgtcgccgtctacatcagagcaggacttatctttgccttccccaaaaattgccactccgtcaaatagattcttttgaatccttgactatttttgcctaaataggtttttgttagtttttcttcaaagcccaaaagaaactctatttagattcatccagaaacaatctttttctcaccccatttcgaagtgccgtggagcacagacataaaaagatgactaccgttcaacctacagggccagacaggctcaccctgccgcatattctactggaattcaacgatggctcctcgcagcatgcagtgatcgagctaagcatgaacgaggggattaatatatccacccatgagtggaatccatccactaatgagcaatcgccacgggaagagagagcaccaccccaacaatccaatccatcgcatcatccagaatcatcgaacatagctactcaaagtcccgctcaggaaaccgagactcagcccggcattccaggactagataggcctgcctttgatacctcggcaacggggtcgtcagaacaggttgacccagtacagggaaggatcctggatgatattataggccaatcattaaggacttccgaagaagacgataccgaatcccgccagagaccacgagaccagaagaacattatgatcaccgtgaattacttgtacgcagacgacacaaattccagaagtgctaatacaaacaaccagacgcccaataacacttctagaacttccgacagtgaacgtgtgggctccttatcgttgcacgttccggatctaccagataatgccgacgattactatatcgatgtactcattaaactaaccacaagcattgccctcagcgtcatcacgtccatgatcaagaaacgattagggcttagcaggga

SEQ ID NO: 78 - DNA, 1157 bp, synthetic construct
  misc_feature 1..1157: note = Description of Artificial Sequence: Synthetic polynucleotide
  source 1..1157: mol_type = other DNA
tactacaggctggctgttcctcgcatggtgtttaatgtcctgactgggttttcgtttatcggtattaccggagccaccttgactgtaagggaacgatactggactaagagagtaatgcgaaaggcaacagcgtttctggcgaacctaatcaatgacggttacgagtttactactcctaaagccagtcttattttgctagagcgagtcaacgcttacttaaagggccagggacctaattatgacatcgattttgacgagcaggaggcgttcattaaagaaatggaggagttgaggacctctggtggatatgagaacagatactcatattcaggaaccgatgaaacacccagagatccgggttgcctgtttcttcccattgctttaaataaatggcactttgatgtgctagactgcctgaggatatacggtactcaggaagatctggaatctaaattattaagtgttcagcaattggtgttacaatgttgcatgaagcacagtggcatgactccagacatggtctttgcaacggaagtagctcagaagccgaccttcgaagacgacatagtttgtgatgatattgacgcttatgcccaggggggtgattgtctagattattgttacacgccaagcaattactccagaactttagaaattcatggcaagattgctaccttacaacgagagctggggctatgctataatattctcggaattttggaccgtttttccgattaaggtttttagctccattgcgccaacccccgctctccagactccttcgttatccagcattcagcatggacaggttcaaaaaataaaatttcttgatatgggtccacttcaaacatgcgcctacctgtaggaaaaaaaaagagaacataaatatgccgcgaacagaaaacgtaatgtactgttctatatataaactgttcagatcaatcataaattctcagtttcaaactttccgctcagccagattttattcgtaaagaacgcatcattggctctatgttgaaggatcagttcttgttatgggttgctttgatagcgagcgtaccggtttccggcgtgatggcagctcctagcgagtccgggcataacacggttgaaaaacgagatgccaaaaacgttgttggcgttcaacagttggactt

SEQ ID NO: 79 - DNA, 1089 bp, synthetic construct
  misc_feature 1..1089: note = Description of Artificial Sequence: Synthetic polynucleotide
  source 1..1089: mol_type = other DNA
ggtccccttcagctacctttctctctgtttggtagttattctcggcgtgtgtatagtatagtataaaagggcctacattggataggcttcaacattcctcaataaacaaacatccaacatcgcgcattccgcatttcgcatttcacatttcgcgcctgccttcctttaggttctttgaatcatcatcaatcgtcgccgtctacatcagagcaggacttatctttgccttccccaaaaattgccactccgtcaaatagattcttttgaatccttgactatttttgcctaaataggtttttgttagtttttcttcaaagcccaaaagaaactctatttagattcatccagaaacaatctttttctcaccccatttcgaagtgccgtggagcacagacataaaaagatgactaccgttcaacctacagggccagacaggctcaccctgccgcatattctactggaattcaacgatggctcctcgcagcatgcagtgatcgagctaagcatgaacgaggggattaatatatccacccatgagtggaatccatccactaatgagcaatcgccacgggaagagagagcaccaccccaacaatccaatccatcgcatcatccagaatcatcgaacatagctactcaaagtcccgctcaggaaaccgagactcagcccggcattccaggactagataggcctgcctttgatacctcggcaacggggtcgtcagaacaggttgacccagtacagggaaggatcctggatgatattataggccaatcattaaggacttccgaagaagacgataccgaatcccgccagagaccacgagaccagaagaacattatgatcaccgtgaattacttgtacgcagacgacacaaattccagaagtgctaatacaaacaaccagacgcccaataacacttctagaacttccgacagtgaacgtgtgggctccttatcgttgcacgttccggatctaccagataatgccgacgattactatatcgatgtactcattaaactaaccacaagcattgccctcagcgtcatcacgtccatgatcaagaaacgattagggcttagcaggga

SEQ ID NO: 80 - DNA, 3964 bp, synthetic construct
  misc_feature 1..3964: note = Description of Artificial Sequence: Synthetic polynucleotide
  source 1..3964: mol_type = other DNA
gccttctcgtgcaatcagagctgttgaaagagagaagagggcacacggaagctgctgttcaattgtgtgaattgaccggattacaacctgctggagtgataggagagctggttcgtgacgaggacggctctatgatgcgattagacgactgtgttcagtttggtctccgccacaacgtaaaaattatcaaccttgaccagatcattgaatacatggattccaagaacagctagatacgatggataggaatacagagatatcatgattgaggaacgtaagagctttttcgaaagtgtgagtttgtggtgagggccaggcggtggggaggtggtggggagcctccttggtcgaatgtagatatagtaagcaagacacaagagcgcgcgaagtcttcaacgaggcggcgttgggtcttgtacgcaacgtaatgactacacagttgagcttgtcgcgaaccggtcgacattttgatcatgcatactatgttgagacaccatctcgtactattgcggcaaccagctgtaaatttgactaattaaagctgatgaaggatgcagggcgtcgtcaattttttgattgattgcatttaattgtttgagccattcaaggctgaatgcccggcaccctagacccttcttgtgagtactataaacccgcaggcagggtacccttggccttctgcgagactaccagtcataacgtatatccacaatgtactagtaatagccccggaaaactctaatcccacagaacgtctaacgcctcctatgtcatcgatacccattcgcactactgccatggccccccttacgtgatcatttcacttactcccgcctaagcttcgcccacatgcctgcgttttgccaagatttactgacgagtttggtttactcatcctctatttataactactagactttcaccattcttcaccaccctcgtgccaatgatcatcaaccacttggtattgacagccctcagcattgcactagcaagtgcgcaactccaatcgcctttcacttcagagtacagaagattaagtgagagaattctaccgttcgtatagcatacattatacgaagttatttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatgactactcttgatgacacagcctacagatataggacatcagttccgggtgacgcagaggctatcgaagccttggacggttcattcactactgatacggtgtttagagtcaccgctacaggtgatggcttcaccttgagagaggttcctgtagacccacccttaacgaaagttttccctgatgacgaatcggatgacgagtctgatgctggtgaggacggtgaccctgattccagaacatttgtcgcatacggagatgatggtgacctggctggctttgttgtggtgtcctacagcggatggaatcgtagactcacagttgaggacatcgaagttgcacctgaacatcgtggtcacggtgttggtcgtgcactgatgggactggcaacagagtttgctagagaaagaggagccggacatttgtggttagaagtgaccaatgtcaacgctcctgctattcacgcatataggcgaatgggtttcactttgtgcggtcttgatactgctttgtatgacggaactgcttctgatggtgaacaagctctttacatgagtatgccatgtccatagcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctataacttcgtatagcatacattataccttgttatgcggccgcaagaagttgattgagactttcaacgagctggctctgcttctggtacttcttcaggtgcatcttctgctactcaaaatgacgaaacatccactgatcttggagctccagctgcatctttaagtgcaacgccatgtctttttgccatcttgctgctcatgttgtagtagactttttttttcactgagtttttatgtactactgattacattgtgtaggtgtaatgatgtgcactataatactaatatagtcaaaatgctacagaggaaagtgcaggttgcctgtggtggtttttcttattagcaccctctgaacactctttacctctaacatcctcagccatgctaatcgcgcataaaataaatcttcgaacttttttccattttatgctcataaagcttccttactgtcaccttatcaaaagagcttttgccactaaagtagtcacacccagaattgctcccgaatatcgtccaacaatgctaggatctgtggaaagtttgacaaataatttgaacaccttgagcttgaagcttcctgaagttaatatccaaggctcctttccagaaagtaacccagtggaccttttgagaaactacatcactcaagaacttagtaaaatttctggagttgacaaagaattgattttcccagccttggaatggggtaccacactggaaaaaggtgatcttttgatcccagttcctcgtctgagaataaagggtgctaatcctaaagatttagccgaacaatgggctgctgcattcccaaagggtggatatcttaaagacgttattgcgcaaggacctttcttgcagttcttttttaacacatcggttctgtacaagttggtgatatctgatgctctggagagaggcgatgactttggtgcacttcctctaggaaagggacaaaaagttatagtggagttttcttctccaaatattgccaaacctttccacgctggccatcttagaagtacaatcatcggtggttttatttccaatctgtatgaaaagctgggtcatgaagttatgaggatgaattatttgggagactggggaaaacaatttggtgttcttgcagtaggatttgagcgttacggtgatgaggcaaaattaaagactgatccaatcaaccatttgtttgaggtctatgttaaaatcaaccaagatattaaggctcaatcagagtctactgaggagattgcagaagggcaatcattagatgaccaggcaagagcttttttcaagaaaatggaaaatggcgacgaatcggctgtaagcttgtggaaaagattccgtgagttatccattgagaagtacattgatacttatgcccgcctcaacatc

SEQ ID NO: 81 - DNA, 1019 bp, synthetic construct
  misc_feature 1..1019: note = Description of Artificial Sequence: Synthetic polynucleotide
  source 1..1019: mol_type = other DNA
gccttctcgtgcaatcagagctgttgaaagagagaagagggcacacggaagctgctgttcaattgtgtgaattgaccggattacaacctgctggagtgataggagagctggttcgtgacgaggacggctctatgatgcgattagacgactgtgttcagtttggtctccgccacaacgtaaaaattatcaaccttgaccagatcattgaatacatggattccaagaacagctagatacgatggataggaatacagagatatcatgattgaggaacgtaagagctttttcgaaagtgtgagtttgtggtgagggccaggcggtggggaggtggtggggagcctccttggtcgaatgtagatatagtaagcaagacacaagagcgcgcgaagtcttcaacgaggcggcgttgggtcttgtacgcaacgtaatgactacacagttgagcttgtcgcgaaccggtcgacattttgatcatgcatactatgttgagacaccatctcgtactattgcggcaaccagctgtaaatttgactaattaaagctgatgaaggatgcagggcgtcgtcaattttttgattgattgcatttaattgtttgagccattcaaggctgaatgcccggcaccctagacccttcttgtgagtactataaacccgcaggcagggtacccttggccttctgcgagactaccagtcataacgtatatccacaatgtactagtaatagccccggaaaactctaatcccacagaacgtctaacgcctcctatgtcatcgatacccattcgcactactgccatggccccccttacgtgatcatttcacttactcccgcctaagcttcgcccacatgcctgcgttttgccaagatttactgacgagtttggtttactcatcctctatttataactactagactttcaccattcttcaccaccctcgtgccaatgatcatcaaccacttggtattgacagccctcagcattgcactagcaagtgcgcaactccaatcgcctttca

SEQ ID NO: 82 - DNA, 1322 bp, synthetic construct
  misc_feature 1..1322: note = Description of Artificial Sequence: Synthetic polynucleotide
  source 1..1322: mol_type = other DNA
ctggctctgcttctggtacttcttcaggtgcatcttctgctactcaaaatgacgaaacatccactgatcttggagctccagctgcatctttaagtgcaacgccatgtctttttgccatcttgctgctcatgttgtagtagactttttttttcactgagtttttatgtactactgattacattgtgtaggtgtaatgatgtgcactataatactaatatagtcaaaatgctacagaggaaagtgcaggttgcctgtggtggtttttcttattagcaccctctgaacactctttacctctaacatcctcagccatgctaatcgcgcataaaataaatcttcgaacttttttccattttatgctcataaagcttccttactgtcaccttatcaaaagagcttttgccactaaagtagtcacacccagaattgctcccgaatatcgtccaacaatgctaggatctgtggaaagtttgacaaataatttgaacaccttgagcttgaagcttcctgaagttaatatccaaggctcctttccagaaagtaacccagtggaccttttgagaaactacatcactcaagaacttagtaaaatttctggagttgacaaagaattgattttcccagccttggaatggggtaccacactggaaaaaggtgatcttttgatcccagttcctcgtctgagaataaagggtgctaatcctaaagatttagccgaacaatgggctgctgcattcccaaagggtggatatcttaaagacgttattgcgcaaggacctttcttgcagttcttttttaacacatcggttctgtacaagttggtgatatctgatgctctggagagaggcgatgactttggtgcacttcctctaggaaagggacaaaaagttatagtggagttttcttctccaaatattgccaaacctttccacgctggccatcttagaagtacaatcatcggtggttttatttccaatctgtatgaaaagctgggtcatgaagttatgaggatgaattatttgggagactggggaaaacaatttggtgttcttgcagtaggatttgagcgttacggtgatgaggcaaaattaaagactgatccaatcaaccatttgtttgaggtctatgttaaaatcaaccaagatattaaggctcaatcagagtctactgaggagattgcagaagggcaatcattagatgaccaggcaagagcttttttcaagaaaatggaaaatggcgacgaatcggctgtaagcttgtggaaaagattccgtgagttatccattgagaagtacattgatacttatgcccgcctcaacatc

SEQ ID NO: 83 - DNA, 66 bp, synthetic construct
  misc_feature 1..66: note = Description of Artificial Sequence: Synthetic oligonucleotide
  source 1..66: mol_type = other DNA
atgttcagcttgaaagcattattgccattggccttgttgttggtcagcgccaaccaagttgctgca

SEQ ID NO: 84 - protein, 22 aa, synthetic construct
  VARIANT 1..22: note = Description of Artificial Sequence: Synthetic peptide
MFSLKALLPLALLLVSANQVAA

SEQ ID NO: 85 - DNA, 60 bp, synthetic construct
  misc_feature 1..60: note = Description of Artificial Sequence: Synthetic oligonucleotide
  source 1..60: mol_type = other DNA
atgaaagcattcctgttgttactacttttactaggcctgtccactacactcgctaaggca

SEQ ID NO: 86 - protein, 20 aa, synthetic construct
  VARIANT 1..20: note = Description of Artificial Sequence: Synthetic peptide
MKAFLLLLLLLGLSTTLAKA

SEQ ID NO: 87 - DNA, 144 bp, synthetic construct
  misc_feature 1..144: note = Description of Artificial Sequence: Synthetic polynucleotide
  source 1..144: mol_type = other DNA
atggaaggtggcgaagaagaagttgagcgcattcctgatgaacttttcgatacaaaaaagaagcatttgttagataagctcataagggtcggaataatccttgtactcctgatatggggcactgttttgttgctaaaaagtatt

SEQ ID NO: 88 - protein, 48 aa, synthetic construct
  VARIANT 1..48: note = Description of Artificial Sequence: Synthetic polypeptide
MEGGEEEVERIPDELFDTKKKHLLDKLIRVGIILVLLIWGTVLLLKSI

SEQ ID NO: 89 - DNA, 57 bp, synthetic construct
  misc_feature 1..57: note = Description of Artificial Sequence: Synthetic oligonucleotide
  source 1..57: mol_type = other DNA
atgagatttccttcaatttttactgctgttttattcgcagcatcctccgcattagct

SEQ ID NO: 90 - protein, 19 aa, synthetic construct
  VARIANT 1..19: note = Description of Artificial Sequence: Synthetic peptide
MRFPSIFTAVLFAASSALA

SEQ ID NO: 91 - DNA, 5679 bp, synthetic construct
  misc_feature 1..5679: note = Description of Artificial Sequence: Synthetic polynucleotide
  source 1..5679: mol_type = other DNA
ggcgcgccgtttaaaccctccaccagccatataccactacaacaccacagaagagaaagagctcatatcatccgtcatgagagagtaccagcacagaaatacagtcaagtaaaactagtatgcaagcattacgtaataatagcaactttatgacaaatcattccatttttttccactggagcgtgcactgcgtaaatcattctctttggaaggcaagggaagaacaacaaaatttttccttccgttatacaaacattgaatcatgtctactgaacccacttttaaattggtccttgtcggtgatggtggtaccggtaaagtaagtgcaaattatttgatgagtcggataatgttttccgccccttagttcccctcatgattactaacaattcatagaccaccttcgttaagagacaccttactggagagttccgtaagaagtacattgctactttgggagtcgaagttcatcccttgtcattccacactaactgtggtcctatcacattcaacgtttgggacactgctggacaagagaagtttggtggactgagagatggttattacattaacggtgactgtggtatcatcatgttcgacgttacatcgagaattacttacaagaacgttccaaactggcaccgtgacttggtcagagtgtgtgagaacattccaattgtgctttgtggtaacaaggttgatgtcaaggaaagaaaggtcaaggctaagaccatcactttccacagaaagaagaacttgcaatactttgacatttctgccaagtccaactacaactttgagaagccattcttgtggttagctagaaagttgtctggtgagccccaattagagttcgttgctgctcccgacttgcaagccccagaggttcaaattgatgccgatttaataaagaagtacgagcaagagaacgccgaggctgccgctatgccattgcctgatgaagatgatgccgacttgtaagcttttacttacagtacattgagaaccatacatagggcacgtatcgtaagtttagttgtttgctgatgtaagctagtttgtttctgtagtgtttcgaggtcgcagagggatctctctagccttagacaaaaaaaaaaaggttgacacgttgatacactctctgtttcatccgatctttcacctacgagtcccactcctcttcagagtacagaagattaagtgagagaattctaccgttcgtatagcatacattatacgaagttatttcagtaatgtcttgtttcttttgttgcagtggtgagccattttgacttcgtgaaagtttctttagaatagttgtttccagaggccaaacattccacccgtagtaaagtgcaagcgtaggaagtccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatggctaaactgacctctgctgttccggttctgaccgctcgtgacgttgctggtgctgttgagttctggaccgaccgtctgggtttctctcgtgacttcgttgaggacgacttcgctggtgttgttcgtgacgacgttaccctgttcatctctgctgttcaggaccaggttgttccggacaacaccctggcttgggtttgggttcgtggtctggacgaactgtacgctgaatggtctgaagttgtttctaccaacttccgtgacgcttctggtccggctatgaccgaaatcggtgaacagccgtggggtcgtgagttcgctctgcgtgacccggctggtaactgcgttcacttcgttgctgaagaacaggactaacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctataacttcgtatagcatacattataccttgttatgcggccgcagatctaacatccaaagacgaaaggttgaatgaaacctttttgccatccgacatccacaggagagttatagagctgctatgcgtgtaaaaatagtttaaatcttcgtaaagtatgttagtccatgtaatttgctatgaatcgatacgctaatctggatgctgaacggatgcttactggcatgcattattcattacccatctaagctgcgccacaacccagtaaattgcagtgagggaagcttccctgtaaccgtcctgtccctttagggaccatcgatccccaacgatcaaatcgcgatacatctatcaactgtccctttccatctatctatgcaaggtaatgacagactctgttaactctgatgattctgatctggaaatcatagaggtgactgagcctactccaaaagtggaccttttggcccccaatccagcatttaattttactgcccccataagcaacagtaacggcacaactccaataaggagaaaacttgatgaccaatccaactccaattcttttgccagactggaatcgttacgggaatcatcagtgaaaccacaagctagtacgttcaatagtagtaggttcatcccccaagccgaccaattttccaataatcagaataatgaacttgataacaacaatggattcgccgactggatttctaagtcccaacctgaatttccctttccacttaatgatggaccaaaaaagtccagcaatcaacctacaaactcaaattttgaagagatcatcgatttaactgaagatatcgagataaatacatctgtccccgcatctacatcatcttctaccccagttccctccagcacacagaatcagagccatcatatagccaacaacaacacagcacaagatgcgcatatcttccaagggaaacgacctctccaatcatattcagatgatgaagacgaagatttgcaaattgtaggatccaatattgttcagcagcctctaggaattatgccaggaactttcaacgcccctgcaaacatactccattttgacggttcaaaccagaatgaacaagccagatggctggacttgcggataaaagatttgttagataatcttcacaatcttcgagttcatgctcagtcgaatattatggagatcaataggttcatttccactttggggcatttaaacagagaagtttaaaccctgcagggcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgcgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 92 - DNA, 1157 bp, synthetic construct
  misc_feature 1..1157: note = Description of Artificial Sequence: Synthetic polynucleotide
  source 1..1157: mol_type = other DNA
aaaccctccaccagccatataccactacaacaccacagaagagaaagagctcatatcatccgtcatgagagagtaccagcacagaaatacagtcaagtaaaactagtatgcaagcattacgtaataatagcaactttatgacaaatcattccatttttttccactggagcgtgcactgcgtaaatcattctctttggaaggcaagggaagaacaacaaaatttttccttccgttatacaaacattgaatcatgtctactgaacccacttttaaattggtccttgtcggtgatggtggtaccggtaaagtaagtgcaaattatttgatgagtcggataatgttttccgccccttagttcccctcatgattactaacaattcatagaccaccttcgttaagagacaccttactggagagttccgtaagaagtacattgctactttgggagtcgaagttcatcccttgtcattccacactaactgtggtcctatcacattcaacgtttgggacactgctggacaagagaagtttggtggactgagagatggttattacattaacggtgactgtggtatcatcatgttcgacgttacatcgagaattacttacaagaacgttccaaactggcaccgtgacttggtcagagtgtgtgagaacattccaattgtgctttgtggtaacaaggttgatgtcaaggaaagaaaggtcaaggctaagaccatcactttccacagaaagaagaacttgcaatactttgacatttctgccaagtccaactacaactttgagaagccattcttgtggttagctagaaagttgtctggtgagccccaattagagttcgttgctgctcccgacttgcaagccccagaggttcaaattgatgccgatttaataaagaagtacgagcaagagaacgccgaggctgccgctatgccattgcctgatgaagatgatgccgacttgtaagcttttacttacagtacattgagaaccatacatagggcacgtatcgtaagtttagttgtttgctgatgtaagctagtttgtttctgtagtgtttcgaggtcgcagagggatctctctagccttagacaaaaaaaaaaaggttgacacgttgatacactctctgtttcatccgatctttcacctacgag

SEQ ID NO: 93 - DNA, 1125 bp, synthetic construct
  misc_feature 1..1125: note = Description of Artificial Sequence: Synthetic polynucleotide
  source 1..1125: mol_type = other DNA
ggagagttatagagctgctatgcgtgtaaaaatagtttaaatcttcgtaaagtatgttagtccatgtaatttgctatgaatcgatacgctaatctggatgctgaacggatgcttactggcatgcattattcattacccatctaagctgcgccacaacccagtaaattgcagtgagggaagcttccctgtaaccgtcctgtccctttagggaccatcgatccccaacgatcaaatcgcgatacatctatcaactgtccctttccatctatctatgcaaggtaatgacagactctgttaactctgatgattctgatctggaaatcatagaggtgactgagcctactccaaaagtggaccttttggcccccaatccagcatttaattttactgcccccataagcaacagtaacggcacaactccaataaggagaaaacttgatgaccaatccaactccaattcttttgccagactggaatcgttacgggaatcatcagtgaaaccacaagctagtacgttcaatagtagtaggttcatcccccaagccgaccaattttccaataatcagaataatgaacttgataacaacaatggattcgccgactggatttctaagtcccaacctgaatttccctttccacttaatgatggaccaaaaaagtccagcaatcaacctacaaactcaaattttgaagagatcatcgatttaactgaagatatcgagataaatacatctgtccccgcatctacatcatcttctaccccagttccctccagcacacagaatcagagccatcatatagccaacaacaacacagcacaagatgcgcatatcttccaagggaaacgacctctccaatcatattcagatgatgaagacgaagatttgcaaattgtaggatccaatattgttcagcagcctctaggaattatgccaggaactttcaacgcccctgcaaacatactccattttgacggttcaaaccagaatgaacaagccagatggctggacttgcggataaaagatttgttagataatcttcacaatcttcgagttcatgctcagtcgaatattatggagatcaataggttcatttccactttggggcatttaaacagagaagttt

SEQ ID NO: 94 - DNA, 1292 bp, synthetic construct
  misc_feature 1..1292: note = Description of Artificial Sequence: Synthetic polynucleotide
  source 1..1292: mol_type = other DNA
ttcagtaatgtcttgtttcttttgttgcagtggtgagccattttgacttcgtgaaagtttctttagaatagttgtttccagaggccaaacattccacccgtagtaaagtgcaagcgtaggaagtccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatggctaaactgacctctgctgttccggttctgaccgctcgtgacgttgctggtgctgttgagttctggaccgaccgtctgggtttctctcgtgacttcgttgaggacgacttcgctggtgttgttcgtgacgacgttaccctgttcatctctgctgttcaggaccaggttgttccggacaacaccctggcttgggtttgggttcgtggtctggacgaactgtacgctgaatggtctgaagttgtttctaccaacttccgtgacgcttctggtccggctatgaccgaaatcggtgaacagccgtggggtcgtgagttcgctctgcgtgacccggctggtaactgcgttcacttcgttgctgaagaacaggactaacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagct

SEQ ID NO: 95 - protein, 124 aa, synthetic construct
  VARIANT 1..124: note = Description of Artificial Sequence: Synthetic polypeptide
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 96 - DNA, 11322 bp, synthetic construct
  misc_feature 1..11322: note = Description of Artificial Sequence: Synthetic polynucleotide
  source 1..11322: mol_type = other DNA
ccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggtgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataacattactcgcatccattctcaggctgtctcgtctcgtctccaactttctgccgccaatctcctttccattcaaattctctacaacaaggcaggcttcgtccgttgatgtgaagtttgcaaaagccaaaccacgaaaaacaccattgtcaaaatggtagttgaaggcataaggcaaaggcaagctaaactttgtcataacgtctaaaagttgctctttttttatggcaaaagggatgttctttatcacaatagcagtgggaatgacatcttcatcatcattcgtatcatcaataccatcgagttctgaatccaaaggtttctgttgttgggataaggcatcagattctttgagcaggtcgtccttggcgtcctcctctagatgaaatttgccatcgttaggaggtgagtattgggaaacgttgacttgttgctcaatccaaggatcaacgttgaccatcggattgccgttctgaggagacagccaggcattataccgtggttgaccggcgtttacgcgcagaggattgttgctggtgttgttgacagaagacgcatacccagacgctgcggatgacatcgacgaaattgaagggcgacgttgcatacccatttgttgctgatccaagaaacttggtgtttcagtcaattgtctcaggtccatattgatcaatgtgttcaactgtttcaatctggttccggagggtggctattacgacaagctgtggctactatctaagtgggagaagtaacggaacacattgatgagacacaggaattacagggcgtgcatccaccaataacaattagtcgagatttcaaccaattacgtaagcgctcaacccttttttcgaacacgtatcgagcaaagtccaggtgaaaccttcatccattatatccaaagtcgaccgaagctttaacaacatccataatggtaagtgtccagtttgatgagtgcagaatggttccaagttttagaccagttactaatatttaaagtctacagcaattccaggaggacagagaacgttagctaaaagaagagcagcaaacttggataagaaacaggatgaaccaacctccgccagatctgccggtgctggaggttcttcgtctaccatgctaaagttgtacacagacgaggcccaaggtttgaaagttgatcctttaattgttcttgttcttgctgttggtttcattttcagtgtcattggtttgcacgttgttgctaagctgacaggaaagttgatcaactaagacctatatttaaacaggtttcatcatatctgtactatatttacaagtccactgcgtttaggtatatactaaagacattcaagaagcacatccacaacttgtgcaagtcctgtcaaatgtactagatgcttttcagaacatcctgcggtttgaggagattcctgaatttcccagtcccaagtctttctcttgtagaggtctttgagttcttgtgaatgctgaattggggttcttacctcaatttctattagtgggaaatgctttcccacaattatttgcaatgggatcccggcaactttactttgcttcaacttatgtcccatactgaactttccgtcacggttgtcaacttgaacgtcgaatgagctcagtatttcggtgacagtgtctagattctttttacttgatgttttattcgaaagtagcgtgatttgatatggtgcgataatttgtggccagggctgggagttcgtagacggaaacaaacgcagaatccaagcagacaagtggagagtggacaaattcttgatgttaattcgtaagatccttaacaaatctttagcaaaactgaaaagtgaagactacgatagggatttacttgaaaaatatattcaagttttgtcagaatacccactccatattaacgatattattgtccctagaactattacgtaccacttgtgtgatatttatactgatgagttggaaaaggttatgtttagtgggcttcctggatttgaagaagaagaagattacgaggaagaagatgaagtctcggctcctatcgaaaaaaaaaccagagatactgataattcagacgatgaggcctctgataccgatccagaaacgagtgacgaagaagacgaaggtgagaatactgaaaacgagtcagaagaggaaccgatcaaactctctgcggaagaagaaacggctctgtggaaaacaaagatggagattatccatgaaactcctatcgacaaattactctcaccttttgtctcattgaagaaagatacctcaaataaaccattaaaattgaaaatccaagaagcagttcttgctgatccaagactcggcaaatggaaagttaaatcgtaccgaaagcctaaaccaaaaccaaaacctcttcaggtgctacagaaacagttatacgaacagatcaaatataaaaagggtaaagcagtcacaggtgaagacgacgatgaactcaaagacgaagacgaagatgacgatgacgaggtcatctcggagagtgaagctgataactctgatgaagaagaagatgaagaatggaatggctttggaactatctagaatacatatgtaaaaccatatcaggcaataacaatttctcgctattttgcatcccaacacctcgaccgacgtgttcagttcgaccctttacctacagtgtctttaactcccatttggatctcctaaataacctacacaaatgtcccaaaaagtcaccgacgtccctctggaatttgttaaggaaggttccaaattcatctctaaatgtactaaaccctctcagaaggagtacttaaagatagtaagagctgttggagttgggtttttaatgatgggcgtggttggttacgttgtcaagctcattcatattccaatcagatatttgattgtttaaaagtttaggtttgaatacaatgtgtatgcttaattatattcacttcgtttcattgatttttgctatccctgttgtgcgttaatcatctctatcgtgatcctctcaagttgcacctcaaatagaagacaacttatggaggtgtactacccaatatcagtcttgacgtttctagtttcgttgtatgctgcataccaatttcagttcttccgtagtgttttgagcataggcttgtttacccgtcttctttacactctgatatgagcaccttatcaaacctcttatcacagctgaaaggagaaggcggtggtggttcttctggtcagaatcggcccagaactgtggatcctgctgttgcaagattgaaagcagaaaggaagatggaaagagagaagcaagctcttaaagaggctcagcaagcccaggaagctcgggaaaggcgaagaattagtcatgctacccagataggtttgagaccaaccagtaaagctcccagcactgaaggtcctcaatcgcactggaaacatcaaggtcgatgaccgcactaacagaaggagctaaactattcgaaaaggagattccttacattacagaattagagggtgatgtcgaaggaatgaaattcattatcaagggcgagggtactggtgacgctactaccggtacgattaaagcaaagtacatctgtacaacaggtgaccttcctgttccgtgggctactctggtgagcactttgtcttatggagttcaatgttttgctaaatacccttcgcacattaaagactttttcaaaagtgcaatgcctgagggctatactcaggagagaacaatatctttcgaaggagatggtgtgtataagactagggctatggtcacgtatgaaagaggatccatctacaatagagtaactttaactggtgaaaacttcaaaaaggacggtcacatccttagaaagaatgttgcctttcaatgcccaccatccatcttgtacattttgccagacacagttaacaatggtatcagagttgagtttaaccaagcttatgacatagagggtgtcaccgaaaagttggttacaaaatgttcacagatgaatcgtcccctggcaggatcagctgccgtccatatcccacgttaccatcatatcacttatcataccaagctgtccaaagatcgtgatgagagaagggatcacatgtgtttggttgaagtggtaaaggccgtggatttggatacttaccaatgactgacctcctgccagcaatagtaagacaacacgcaaagtctctgaacgggtctttgagctgtctgtgtcatcaaacatatcttcatctgtagttgtattgttttcttttatcactaggagtccatccttctggatggctgtcttgcacttatttagccactccaaaaatgccgggtctgggagatgaccgcagcaccattgacaccatatcagagagtatcgagactcctctggtttccagtcttgcatgggaatttcataaatgtctcctatcttaccttgttccatcagtgtttgtagttccaccctcatctggtcgacaaatggtttaactggttccaataaatcaaccttatcacaaactttatgcagaaaatctcttgtcacgcggccaataccggcaccaaagtctataccatatttgattttgtcaggatcgttagagaaacgactctttaactttcttaaaaaggtcatcgatccaacaacatctgcttttggaaccgaagttgtctcaccgtacccacccaaaactccgtcgacagatgctgggacgctgttccagtatttcagggcatcgtcatagttgatcaaactgtccacctgtttagggtcatccccattatgttctgtcattgtggtaaaaatgggatacagtgatatattgaaggggaatggttataagagcctacctgagaaataaaattattatgcgccatccgacatccagaaaaattgatgaaagattggctattgttgacggttcttgatcccaaaaaaaaaaaaaaacaagaaatgctgccgtcctagttttgcttcaaagaatggtttcgtgctatgccattcccaacccaaagagctgtcccatcccattaagttgtgctgactgattatgttgcacaatccagtgtcgtgattacctccaacatcgcacgcgaatttcgccatggctgggaaacccaaattcttcggtcgtccatcaaactctgaagtcatttcaacaccaaactcaacagctatcatagaaaaatatggcggggttgcgttttttagacattgcaagaccatttgtcagctggatcccggaagttgaacttccttatgaaaactgggggttcgatgaaaagctgatttactcatttttcactgctgccatctatttgattctgtccctgcctatatacggtgtcaaatcctctgaagtcgtggacccagttccccatttgcgttctgccttagggagtgagaagggaacattgctggagcttgggttactgcctgtgattacttcggcatttatcttgcagttgttggctggttggaaagttttcaaagtaaactttgatctggttagtgacagaatattgttccaaactttgcaaaagatcacttcagtcgttatcagcatcgtatatgctgttcttctcacattttgtgactactttactccaggtgtgtccactgataacgtcttgtggtcccaatttctgatcatcttacagatagtggtggtcaacttcttggttactctactcgttgaagtcattgacaaggattacggattttcttcaggagctctattgttgcttgcggtttattccgccaccaacttcgtttttggcacgattggtcttagcaccgtcaacacctccagatcgaacgaatctattggtgctctgattcaattattccgcaatttgagctctaaaccaattggtgttgccatatatgactccttcttcagagtaaaccttcctaacttgactcaattttatctggggattgccattatttgtgtttgtctgttcttgaataatgcaagatacgaagtaccaattaagccaaacaaggttcgtgccatggcctcagcttacccaatcaagctacttttcaatggttctttgccacttctgtacacgtggactgtgctgtacaacttgaaccttattggtttctttgtcttcaagcttaccaacttttctcttttagggaacttcaaagtggacccattcggcaacaactactacgaaattacatctggactgctgtatttattgactcctactttcaacgctgaagctggacttttacccaatgttgctaagccatttgttttcattgccttctatgttggtgttagcactttctttgctagatcgtggtccaacattaacgggtcgtcaggcaaggacattgccaagtttttcaaggctcaaggaatctcattgttaggaaaaagagatgcctctgtgtctaaagagtttaacaccctagttcctgttgcttctgcctctggagctttcctattgtcttttccagttgccgtcgctgagttattgggtggctctggtgttccaacctctatcggaatcggtcttttgagtggtttggctattttggaaactgttttgcaagaatggcaacagtctggaggtgcctcacagttctcccaatacttccagacttcttaggtttagaaatccttgaagactatccagacattcacccgcacctcaatttaccttctacatacatcacatattctatagaggagagttccattgctcgtactgaaccccacaccactcttctttataccctacaaactcttcgtccaactcaatggcgtcattcgtgtcggtatagacaataatggtaccccagtccatttcaaagttgtctttttcgatatccatgattaatttgggcattatttgaagttcgaactgttttcctggcactttagctttgatgatcgtttgatatatttcatccttggagttatacagtagtggctttcctcccaggtggtatcgtaaaacctgggaaggattgtgctcaagagccaactccctttacaacctcactcaagtccgttagagggcgcgccgcacatgaagctgtacatggaaggcacggtgaataaccaccacttcaaatgcaccagcgagggtgagggtaaaccgtatgaaggcacccaaacgatgcgtatcaaagttgttgagggtggcccgttgccgtttgcgttcgacattttagcgacgagctttatgtatggctctcgtacgtttatcaagtacccgaagggtattccggactttttcaaacaatcttttccagagggtttcacctgggagcgcgtgactcgctacgaagatggcggcgtcgtgaccgcaacgcaggatacctccctggaagatggctgcctggtctaccacgttcaggtccgtggtgtcaatttcccgagcaatggtccggttatgcagaagaaaaccctgggttgggaaccgaacaccgagatgttgtatcctgcagatggtggcctggaaggtcgcagcgacatggcattgaaactggtcggtggcggccatctgagctgtagcttcgtgaccacgtatcgttcgaagaaaacggtcggtaacatcaaaatgccgggtattcacgcggttgaccaccgtctggtgcgcattaaagaagccgacaaagagacttacgtggagcaacatgaagtagccgttgcgaaatttgctggtttgggcggtggtatggacgaactgtacagttccttatcatctggcgaatcggacccacaagagcactgggttccgttttacattccaggaagagtttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatgactactcttgatgacacagcctacagatataggacatcagttccgggtgacgcagaggctatcgaagccttggacggttcattcactactgatacggtgtttagagtcaccgctacaggtgatggcttcaccttgagagaggttcctgtagacccacccttaacgaaagttttccctgatgacgaatcggatgacgagtctgatgctggtgaggacggtgaccctgattccagaacatttgtcgcatacggagatgatggtgacctggctggctttgttgtggtgtcctacagcggatggaatcgtagactcacagttgaggacatcgaagttgcacctgaacatcgtggtcacggtgttggtcgtgcactgatgggactggcaacagagtttgctagagaaagaggagccggacatttgtggttagaagtgaccaatgtcaacgctcctgctattcacgcatataggcgaatgggtttcactttgtgcggtcttgatactgctttgtatgacggaactgcttctgatggtgaacaagctctttacatgagtatgccatgtccatagcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctccgaataacttcgtatagcatacattataccttgttattacagcggccgcaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcgacgtc

SEQ ID NO: 97 - DNA, 888 bp, synthetic construct
  misc_feature 1..888: note = Description of Artificial Sequence: Synthetic polynucleotide
  source 1..888: mol_type = other DNA
caactttctgccgccaatctcctttccattcaaattctctacaacaaggcaggcttcgtccgttgatgtgaagtttgcaaaagccaaaccacgaaaaacaccattgtcaaaatggtagttgaaggcataaggcaaaggcaagctaaactttgtcataacgtctaaaagttgctctttttttatggcaaaagggatgttctttatcacaatagcagtgggaatgacatcttcatcatcattcgtatcatcaataccatcgagttctgaatccaaaggtttctgttgttgggataaggcatcagattctttgagcaggtcgtccttggcgtcctcctctagatgaaatttgccatcgttaggaggtgagtattgggaaacgttgacttgttgctcaatccaaggatcaacgttgaccatcggattgccgttctgaggagacagccaggcattataccgtggttgaccggcgtttacgcgcagaggattgttgctggtgttgttgacagaagacgcatacccagacgctgcggatgacatcgacgaaattgaagggcgacgttgcatacccatttgttgctgatccaagaaacttggtgtttcagtcaattgtctcaggtccatattgatcaatgtgttcaactgtttcaatctggttccggagggtggctattacgacaagctgtggctactatctaagtgggagaagtaacggaacacattgatgagacacaggaattacagggcgtgcatccaccaataacaattagtcgagatttcaaccaattacgtaagcgctcaacccttttttcgaacacgtatcgagcaaagtccaggtgaaaccttcatccattatatccaaagtcgaccgaagctttaacaacatccata

SEQ ID NO: 98 - DNA, 953 bp, synthetic construct
  misc_feature 1..953: note = Description of Artificial Sequence: Synthetic polynucleotide
  source 1..953: mol_type = other DNA
agacaagtggagagtggacaaattcttgatgttaattcgtaagatccttaacaaatctttagcaaaactgaaaagtgaagactacgatagggatttacttgaaaaatatattcaagttttgtcagaatacccactccatattaacgatattattgtccctagaactattacgtaccacttgtgtgatatttatactgatgagttggaaaaggttatgtttagtgggcttcctggatttgaagaagaagaagattacgaggaagaagatgaagtctcggctcctatcgaaaaaaaaaccagagatactgataattcagacgatgaggcctctgataccgatccagaaacgagtgacgaagaagacgaaggtgagaatactgaaaacgagtcagaagaggaaccgatcaaactctctgcggaagaagaaacggctctgtggaaaacaaagatggagattatccatgaaactcctatcgacaaattactctcaccttttgtctcattgaagaaagatacctcaaataaaccattaaaattgaaaatccaagaagcagttcttgctgatccaagactcggcaaatggaaagttaaatcgtaccgaaagcctaaaccaaaaccaaaacctcttcaggtgctacagaaacagttatacgaacagatcaaatataaaaagggtaaagcagtcacaggtgaagacgacgatgaactcaaagacgaagacgaagatgacgatgacgaggtcatctcggagagtgaagctgataactctgatgaagaagaagatgaagaatggaatggctttggaactatctagaatacatatgtaaaaccatatcaggcaataacaatttctcgctattttgcatcccaacacctcgaccgacgtgttcagttcgaccctttacctacagtgtctttaactcccatttggatctcctaaataacctacacaa

SEQ ID NO: 99 - DNA, 991 bp, synthetic construct
  misc_feature 1..991: note = Description of Artificial Sequence: Synthetic polynucleotide
  source 1..991: mol_type = other DNA
tctgaacgggtctttgagctgtctgtgtcatcaaacatatcttcatctgtagttgtattgttttcttttatcactaggagtccatccttctggatggctgtcttgcacttatttagccactccaaaaatgccgggtctgggagatgaccgcagcaccattgacaccatatcagagagtatcgagactcctctggtttccagtcttgcatgggaatttcataaatgtctcctatcttaccttgttccatcagtgtttgtagttccaccctcatctggtcgacaaatggtttaactggttccaataaatcaaccttatcacaaactttatgcagaaaatctcttgtcacgcggccaataccggcaccaaagtctataccatatttgattttgtcaggatcgttagagaaacgactctttaactttcttaaaaaggtcatcgatccaacaacatctgcttttggaaccgaagttgtctcaccgtacccacccaaaactccgtcgacagatgctgggacgctgttccagtatttcagggcatcgtcatagttgatcaaactgtccacctgtttagggtcatccccattatgttctgtcattgtggtaaaaatgggatacagtgatatattgaaggggaatggttataagagcctacctgagaaataaaattattatgcgccatccgacatccagaaaaattgatgaaagattggctattgttgacggttcttgatcccaaaaaaaaaaaaaaacaagaaatgctgccgtcctagttttgcttcaaagaatggtttcgtgctatgccattcccaacccaaagagctgtcccatcccattaagttgtgctgactgattatgttgcacaatccagtgtcgtgattacctccaacatcgcacgcgaatttcgccatggctgggaaacccaaattcttcggtcgtccatcaaactctgaagtcatttcaacaccaaactcaacagctatcatagaaaaat

SEQ ID NO: 100 - DNA, 460 bp, synthetic construct
  misc_feature 1..460: note = Description of Artificial Sequence: Synthetic polynucleotide
  source 1..460: mol_type = other DNA
gacctatatttaaacaggtttcatcatatctgtactatatttacaagtccactgcgtttaggtatatactaaagacattcaagaagcacatccacaacttgtgcaagtcctgtcaaatgtactagatgcttttcagaacatcctgcggtttgaggagattcctgaatttcccagtcccaagtctttctcttgtagaggtctttgagttcttgtgaatgctgaattggggttcttacctcaatttctattagtgggaaatgctttcccacaattatttgcaatgggatcccggcaactttactttgcttcaacttatgtcccatactgaactttccgtcacggttgtcaacttgaacgtcgaatgagctcagtatttcggtgacagtgtctagattctttttacttgatgttttattcgaaagtagcgtgatttgatatggtgcgataatttgtggccagg

SEQ ID NO: 101 - DNA, 492 bp, synthetic construct
  misc_feature 1..492: note = Description of Artificial Sequence: Synthetic polynucleotide
  source 1..492: mol_type = other DNA
aagtttaggtttgaatacaatgtgtatgcttaattatattcacttcgtttcattgatttttgctatccctgttgtgcgttaatcatctctatcgtgatcctctcaagttgcacctcaaatagaagacaacttatggaggtgtactacccaatatcagtcttgacgtttctagtttcgttgtatgctgcataccaatttcagttcttccgtagtgttttgagcataggcttgtttacccgtcttctttacactctgatatgagcaccttatcaaacctcttatcacagctgaaaggagaaggcggtggtggttcttctggtcagaatcggcccagaactgtggatcctgctgttgcaagattgaaagcagaaaggaagatggaaagagagaagcaagctcttaaagaggctcagcaagcccaggaagctcgggaaaggcgaagaattagtcatgctacccagataggtttgagaccaaccagtaaagctccca

SEQ ID NO: 102 - DNA, 371 bp, synthetic construct
  misc_feature 1..371: note = Description of Artificial Sequence: Synthetic polynucleotide
  source 1..371: mol_type = other DNA
gtttagaaatccttgaagactatccagacattcacccgcacctcaatttaccttctacatacatcacatattctatagaggagagttccattgctcgtactgaaccccacaccactcttctttataccctacaaactcttcgtccaactcaatggcgtcattcgtgtcggtatagacaataatggtaccccagtccatttcaaagttgtctttttcgatatccatgattaatttgggcattatttgaagttcgaactgttttcctggcactttagctttgatgatcgtttgatatatttcatccttggagttatacagtagtggctttcctcccaggtggtatcgtaaaacctgggaaggattgtgctcaa

SEQ ID NO: 103 - DNA, 1487 bp, synthetic construct
  misc_feature 1..1487: note = Description of Artificial Sequence: Synthetic polynucleotide
  source 1..1487: mol_type = other DNA
ttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatgactactcttgatgacacagcctacagatataggacatcagttccgggtgacgcagaggctatcgaagccttggacggttcattcactactgatacggtgtttagagtcaccgctacaggtgatggcttcaccttgagagaggttcctgtagacccacccttaacgaaagttttccctgatgacgaatcggatgacgagtctgatgctggtgaggacggtgaccctgattccagaacatttgtcgcatacggagatgatggtgacctggctggctttgttgtggtgtcctacagcggatggaatcgtagactcacagttgaggacatcgaagttgcacctgaacatcgtggtcacggtgttggtcgtgcactgatgggactggcaacagagtttgctagagaaagaggagccggacatttgtggttagaagtgaccaatgtcaacgctcctgctattcacgcatataggcgaatgggtttcactttgtgcggtcttgatactgctttgtatgacggaactgcttctgatggtgaacaagctctttacatgagtatgccatgtccatagcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagct

SEQ ID NO: 104 - protein, 189 aa, synthetic construct
  VARIANT 1..189: note = Description of Artificial Sequence: Synthetic polypeptide
MTTLDDTAYRYRTSVPGDAEAIEALDGSFTTDTVFRVTATGDGFTLREVPVDPPLTKVFPDDESDDESDAGEDGDPDSRTFVAYGDDGDLAGFVVVSYSGWNRRLTVEDIEVAPEHRGHGVGRALMGLATEFARERGAGHLWLEVTNVNAPAIHAYRRMGFTLCGLDTALYDGTASDGEQALYMSMPCP

SEQ ID NO: 105 - DNA, 207 bp, synthetic construct
  misc_feature 1..207: note = Description of Artificial Sequence: Synthetic polynucleotide
  source 1..207: mol_type = other DNA
gctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaa

SEQ ID NO: 106 - protein, 69 aa, synthetic construct
  VARIANT 1..69: note = Description of Artificial Sequence: Synthetic polypeptide
APVNTTTEDETAQIPAEAVIGYSDLEGDFDVAVLPFSNSTNNGLLFINTTIASIAAKEEGVSLEKREAE

SEQ ID NO: 107 - protein, 8 aa, synthetic construct
  VARIANT 1..8: note = Description of Artificial Sequence: Synthetic peptide
HHHHHHHH